SEQUENCE LISTING (170 sequences)

SEQ ID NO: 1 - protein, 999 aa, unidentified
  source 1..999: note = uncultivated organism; note = M105 -1 Effector
MAETDYNKYVFGAYLNMAFDNFLNTVKLLAAKMKIDYKEDSPASIIDEIFKDENTSPNVENIVEFYFPWMKALMSGLGYDKGYRDLENIKDFYAKVLRAFAYCMNDFRNFYTHTIHVSQKIEPIQYTINGQKKTCRIADALDMIYDSAVNLVKERFVADEADLTHLRRFNSKGKKKTEADGFVYLLHDDSKLTEKGIAFVTCLFLNRKYGYLFLKQLSHFKRSDERRYQFTLETFLAFSNIKPIDKLKSDKLDRFSLGLDMLNEVTKIPQELIKIIPNSFVEKYFSTDKEESFGSRIRYKSRFEQLALEFISQIDEFKDIRFYTYYGSEVKKGYMKQLIDGTETERYLTDSVCGFCRSLNDYTSDDKAREKGEKIKDPNEPGYMLPDAFEPHILRAEPHFIINNNNIGIKIGEADSKGRKDSEKNEEPDFWLSIYELPAMLFYAYLRSNREYKKRCSNRSVRDILEQYRNSKPEPRRRRTDSTEDLMLHRIKKELRWTDTKLAENDRIKNNVAYGKKGHRSNKTGTIADILAHDMIRLQPARCGGDKVTGANFRALQTSLAYFRRDILGDVFKRAMLTAGDHPHPFLNKIDVAHCTSLRDFYVEYLNKRKEYFEKVARKIASGKLNTPCHLLRDLQRSPDDKVSEDRHAPVFLPRGIFTDSIKDCLKKTSLSKVINNARNEKPSINAAFLITQFYTEIQHGKFQEFYNQRRKYDYIDKKNHLTLEERKVAMAKLKPEKIDIRKANDPISKEEYLRRKEYKKVCDNESAIRLYQVQDVLMWLMVKHIFADSLSDISENNISLDSLNDLFEMPVKYTPKFRNITVSANQIKIKDYGKIRRLGRDTKFISLVKAIDKISQKPFTIDYADYLQEEDNFEKYRICMVKICQDIEDRIVKLLDLELAEGDTNFNFNSQIITPYNERYKILSKDEESFIIDTRNMFMHSEYKDLDVNYVIDHDYGGKLTDVFFSEKIYGHFVGLVEKIGKDIEKRTENKALMNDRL

SEQ ID NO: 2 - protein, 1012 aa, unidentified
  source 1..1012: note = M105 -2 Effector; note = uncultivated organism
MEIGSKDQKYIFGAYLNMARDNFSNTIELMAGKIYHHMDSRIEDKNEMEILDDVFGNVSYANLENVVEFYFPWIKALRSPAGLDNGDADLNVMKAFYKSVLRSFFCLVDSLRNEYTHYDHEQVNFGNIKIESVSSAGHRYVCGILNALDKIYDSAVNLAKQRFMADESEVAHLRRYKAVKGKVVRRTVKDKFYYVLSENNNLTEKGLAFVISLFLNRKYGFIFLKALGGFKRSDERSYRMTLETFLAFSNIKPYDRLKSEKMDRASLGLGILNELSKIPKELSDTLSFDYINNYLDTQDEEDVKSRIRYRDRFVPLTLEFISQSDEFKDFMFYTYMGNYVYKGYMKELIDGSSKERYLTDCICGFCRSLNDVSSDYIAEKYNVKIKDSNEPGYTLTDAFEPHILRAKPHFIINNNNIGIKIYDEDRDYNPVISEKGIKCPEPDFWMSIYELPAMLFYAYIRSRREYRNRCIYSVKELVKMSIKSQKDEKKNCEDKESLMLRRVKKEIIWTQSRLDEIKRINESNSAYGKMGHSTLKPGKMADVLSHDIIHLQPAEKSSDKLTGANFQAMQASLAYYRHDILEEVFKRAMLTTGNHEHPFLKKIKLSDCSTLYDFYVTYLKERKIYFDNIERKIYQGKLNTPCHILRRLQKDDNTYQTVGDVPVFLPRGIFTDAIKEFMMKTKLNADIKNARTGKNPSINAAYLILMFYNKIEKGGFQDFYNQPRRYAYFEKNMRLTSEEKKKGKYMSLDERKSALLSLKPQRISTTKANEYISKEEHLSRKGYKEVCDNEATIRMYQIQDILLLFMAKNIFSDVFKDVNSENIGLDKLENLFSKVVSFSKTFENISLNADNVKLKDYGKICRLAVDYKFNSFKKAFHKVHPNHINWNYASYLEEEAAFENYRVCVVKYCHEIEHKIIEVLKLKRVNNTHFNFRKDLITPYNDKYSVFNSEESEFLIQARNMFMHSNYNDECVEYVYRKYFNGSLDDLLFAEKTYKHFVELYDKLKADINQKY

SEQ ID NO: 3 - protein, 939 aa, unidentified
  source 1..939: note = MG103-1 Effector; note = uncultivated organism
MNGAKSKKSVAKANGLKSTFKLGENKYFMTSFGKGNTALEEKDVEESTITDLRNTFKAEFADNDNTTIVINGVAGSAGVQLPCNDANQLRAKSTIEKIVFGETYSDNMHIQIAYSVMNIKKLLAVYANNIIYTVNNLLKTDYNYDFLGMFPTMNDFRKGSIAYNIIKTQLINAERSGITPTNGTPRRGTYFINKDSWKANRKTALKELELLVNQEPDSGIDFWGKLSYCIIHRLGFGNYEHIKKAAESFEKIKNIGLCQYYFSDVFTDKKSEPNMEYIYESLRLLGIMRQTAFHSSEIKKTGNKNSYTLFEIDCCGEKEMKSTLDNITKKKIDALNDKFINTSSVNLNILFSAYPKADKSEIIKEYYDFSVRKVYKNMGFSVKALRETLLATEESIKNLTSKDFDSVRSKLYSLIDFVIYCHYKNNEDEAESFVSALRSLISTKEDEKSGVTYDDKKNELYIKESKRLWKSIDRTVVEHIIPDVEKCKKSENIKPLLSDIERKVKESIRDIIQHYDDLSYFSKAVYCICSFLDGKEINMFLSSLIQDFENIESFKRAIEYTDNDISFSKNFDFYNHCQNIAEELSFIKSIARMNKSNIAVKQSTVKIKSFQYFESAALFGENNQETVKELFKLESGKSPQKGAHVLRNFFINNVINSNKYRYVIRFVNPSNARKIMQNEAIVSFAIKEVDDSQIIRYCNSIGIAYDAENPDLDKMRNELSKKLMKVNFETFAKVSNDRDKNIEKEQLKALIGLYLAIIYLITKSLVRINTSYSIAFSVFERDCYIMSEKSKDKEDEFSALLENVSVKNNPQNITEYFKRSGLLNKRVRNLINRNEKHFNAEVFKYFRNNVEHLSVVSALTNYAEEIKRVESYFDLYHYLMLKIISRTYVGKYIENIDSALDHQTAYKDFLYGILTPFAYNPARYLNLTSKTLFIRGYGK

SEQ ID NO: 4 - protein, 909 aa, unidentified
  source 1..909: note = MG103-2 Effector; note = uncultivated organism
MIVKNLSNSGEKQVLKSKAKSIGVKSTFVLSNKKVIITSFGRGNAAIIEKLIDENSEDKKIIAINPKNAFDVNLLNKEPKVYEIKKGREETKITKPNIKEKVGSDLIGAKDKLEMKIFGRNYDDNVAIQIIYNILDISKIISIYINSAIATMNNLARSSGVEEVDYIGDSVFSDTDYDKMIKQINNERKIDKFTPYYDKCEPRLTYFHAVYEDKIDENKKRVPIDAKYLHETLRVLSAIRVFCFHGKKDNNNSLIFHLEEGPKEEDFPDDLKDIIHNIYKNAVDSANKGFKKSATNINILFKILECNDNNEKKEVAQSFYNHIIIRDSKNQGFSYKKLREELLKNEIYKKINDKKFDPQRGKLYTLLDYIIFWYLEKEKNTDPMIIDKLCNKLRSIRGTNEFAKEKTYNDFANEIAPKLKNYIYRLISFMGAEEIKKFQKMAKKNENEIVRLAEEKIKDSDTTYFSKLIYFISRFLDGKEINEFYGALINKFDNISGIIDAINNKDFNYNEKLEFSNLYSLFKTPHKVAKELRITKNIASLKAIDPPAIHREKYIDAVLLLGLKENDSKGKPYIEEVEVFVDKLLDKSGKDTNLRNFLVNNVISSKKFLYIVKYCNPLTAKEFLKNTNIVEFTISRMDSNLKERYCQAIGISFDENTANSLLTKKIVDLSGEDFLDVKQNPKTNDEKLKKEQYSAILGLYLAVMYVIIKSLVKVNAFYVIAISRLEFDTKMIMGKIPKKTLEKTRDKNGKPINVVIYSAILNDMLEKDNYLKKTKYRHIQQNLGEYSNTLFIEYRNKIAHLSVINLGHKYINGVEKIYSYFGLYHYIMQRILRDSREKIKNYEGSDVENNTINKCFNDLEKFNYFSKDLINILNLPFAYNIARYKNLSIEDLFNSQDEKYIPTEKGRKK

SEQ ID NO: 5 - protein, 909 aa, unidentified
  source 1..909: note = uncultivated organism; note = MG103-3 Effector
MAKKKQSIRDKRNLEKERKAEKFRKMYDAGTAEAVPVPPPKGKRKKSLVKAAGVKSGFAVGSDVYMTSFGRGNSAVLEKKISGRTVVDLPDSKGAFTAEAVSDADIAVTNSRLKYKVANANNPLHHNQGAPAVRQDNLHAKSKLERIFFGEVYSDDNIHIQLIYNILDLEKILAQYVLDAVYLLHNTVKDSDKDDLMGYLSVRNSYYVFMHANGQSEDPIEKQIAEQKAQFLKRLNSGRLGYFGTAFYEAPADKNSIADKQKDTKEIYHIFALIGSMRQSLFHGHRKTKRHQGSAWIYCLEDKLSQEYKDTLDKTYRSAIDRLNRDFGKTNRVNLYIIENALKCLYDSVEPSELAQDYYDFIVKKSYKYLGFSIKHMRETMLENTDAQKYKSKSFDPVRSKLYKLIDFLIYDLYYNRYPERIEDVVCRLRLTMSEEEKNEIYCKEANFVWKKLSDVILNIIGPAVDVNNVGKIKPNNSNKRWNVDKVCIKADTNYFCKLIYVLTLLLDGKEINDLLTTLINKFDNIASFIDVMDKLGLKCEFTPEYKMFADSKKICADLKLINSFSRMQKPNNKSIKVMFKDALEIMGMDISDSDEYIDNMQDRDETGKPKQRGDHNFRNFIINNVITSDRFKYLVKYSNAKTVRKLASNKKIVGFVLNHIPDTQIERYYSSCHGSGAYDINGKRKYLADILHDLDYKDFEGVINNDNDKNVTDQQKDDKSKKQAIITLYFTVLYLAVKNLIYVNSRYVIAFHCLERDYYLHDKSLDDNYSNCFELTDFFIDNGYINTRASQYLKQNISNSNTLAVKKYRNNIDHFAIVRDIGDYIEQIRDFTTWFQLYHYFMQRIVLKENISGFEKYNDMILEHNTYCKNMVKSLNAAFGYNLVRFKALSIDELFDRNNYLPDKTEKN

SEQ ID NO: 6 - protein, 970 aa, unidentified
  source 1..970: note = uncultivated organism; note = MG103-4 Effector
MEKNRKSKAKAMGVKSTFNINGSTVVTTFGKGNDAIVEKTIENGIVKTNTENPSIDLNVIKPDNKEFEVIKGRVSFPDEELPVLYMPKTEDVKINTGDKYVSYKEELEKRFFGRTFNDSIHIQLIYNILKINKILASHSNNATFVVNNLFSTVSGEPGRDIFESISTANTYSFVRNIEVQNLNPKAVESIKTFRETFDKIFDVRNNYSKEKPRRENARLSYYGNAYYKRNGKLRDDEDIFNSYALLGSIRNTVTDSLPSKLFSNILFNLDSNKVDKSFKDTLDRAIEDIDYVNNGFVKNNKVNYYILFSAYNCFSNNELRKQITRLFYEFAVTKAYKNMGFKIKQIRENIIKTAHPELADKKYDSCRSKLYQLLDFVIYYHYLDKDSEVQAIVDLLRASRSNEDKEKIYADEAKRVYGSIKEAFRRVTDLTNPVNIGRIDKNKVHVSEADIAAEKVTGYDYFSKLVYMLTLFLDGKQINDLLTTLIHSFENINAFKHTLSEIGETDPFVFPYEFFNNQKTVRIAKELRFINSVARMKISDPGYKKAQYTDAMIILGTQMSSKEISEEADAFLSKDTDNNLRNFLANNVIESRRYKYLVRYIRPSMAKAVASNPNIVRFVLNDIPEAQIKRYYDSVYMKHIVDRHKKDKYGNPLQRNIPPEPVSADEMRSKLSDVILRVKYDDFKDVRQKVRRGTEDELDKERKKAVIRLYLTVIYIFVKNMVNVNSRYVIAFHCLERDSGIFGYDMREKQYDSDGKPIYNKNYSFLTVVYINTANELYNAELEKPFKQRNKVKRPINQRFCRILENNLVFRAPSYSFTDRYSENPALRSEKRKMIENHMSGDNNDLAQKMQGYFRNNVAHLNVLRTFDSEITSVDGKTSTIISCLGRFNSYFEAYHFLMQLQLTVNASGFIAEKAEVKELRETLLKTGCYSKNTVKTLCAPFGYNLIRYKNLTINELFDKNAVNETDVKA

SEQ ID NO: 7 - protein, 978 aa, unidentified
  source 1..978: note = MG103-6 Effector; note = uncultivated organism
MKKKTSIRDQREQQAAKMRLAHQQKEAVRQETVRAQREAEEAAAREKERIRQAAQKAVRLKELNNDPNYHPIRQRSLSEEKKSTAKAAGLKSTFIMGEKDLLMTSFGKGNAAVVEKKIAAGEVSDLTDKPAFTAVAEKDISFRIDGRVKNASTDNPLHIAEEKTKTREDLIHARSSLEMLYYGENFEDNIHIQAIYSILDVEKILSIHINNIVYMLNNLLREEKEDLMDLIGYLRTNDKPYEKFLEGQGNDGELLTAFRNLCMNKRLGYLNLEVIPKEPPKNKDGKPIRRKADPNTIKMTDEEFYHILVALGKMRNMLAHGNPKQNIYKIDGFGKNDAISAVLDRLYQERIIELNKGFLEKAGKNLAILFHAFSVKEREEKAKYVQDYYDFTVRKNYKNTGFSIKLLREHMTTDVEEAFVLRNKQYDSVRGKLYPFIDFAVYRYYRQHPAEAEELVGSLRASMDEKEKDVIYSREAERIWEKDQVGDLILHHILPEMSGDTIKNLKPDKDVTEEMLQSVLITDEATDFSKMVYMLTLFINGKEINDLLTTLIHQFENIDAFTSVMKSQNLRTGLVKDFQLFNRSAQVAKELRVINSFARMTKEAAFAKEPMYIEAFRVLGMKADEKQLVQEAKDLLDPEKSGAGTQRRGVRNFIANNVIESVRFKYLIRYGNVNKLKAIAQSPAVVSFVLKDIPDDQIRRYCQDLGDRREMDVTAMRSILTERLTGFSFEDIRDVRQNDQKANQKEQQEKQQKQALVRLYLTVLYLALKNLVYINSRYFLAFHCVERDRLVLDYEYWKATLNDEYRNKPGYELSAFAKEFLEKYPQKKRVKAYLELNFANSDDWAISAFRNKVEHLDAIRNADRYIADVKEIRSWFELYHYIMQRRIMDQYEWDSTHESRKEKGTMIISEDRLNPKTLEYFNKVRTYGSCCKDFVKALNVPFAYNLPRYKNLSVDELFDRNRPLAKGKGMGRNLENDE

SEQ ID NO: 8 - protein, 951 aa, unidentified
  source 1..951: note = uncultivated organism; note = MG103-7 Effector
MITKTIESGFIMGDVKTTEKKSIAKANGLKSTFFIDNDKTLMTSFGKGNSAVLEKMIDNYDVEELQDENKKAFDAAVKTLGEAHIKGRCGESDVVLPKSSDTNHLQAKSTIEKIYFGKTFTDNIHIQIAYNIMDIKKIFAIYANTIVESIDNLKDRHTDSVADKDEFLGMLYTFNSVEKSSLASLILKEGLIDEEKSKSYFFIKGNVWKSKKHKQLKALNAKFNYHGDFQNELRSLGTYLQNQFRDLKKMKKAAKSYDDIQKIIKSSIYFGDAFLDQNGNVDVNTAYTIICLLGVLRQQSFHEKSTTNTWIFEIDNKRDQGFNNALDTVFKNKVDKLNNDFLKNSKVNLSVLFEIYGGEKKSELVKEYYDFSVRKSYKNMGFSIKTLRETILTFEDAKVITDQKYDSVRSKFYSLFDFVIYRYYLANQKRGEDFVDKLRKCVTKEDKPNLYENEAKMLWTNIKTSVLKQIMPKMDGNYIKKIAKNEDVASQYSSALDGIMQTAENTSYFSKTMYCVSLFLDGKEINCLLTSLINKFDNINSLCRVLVDAGYESDSNIFTDKYMLFADSEKITDELRFVNSFARMSKTTKAVKDGNASVNRQMYYDSAALFGEFNKEKVEKLYHLGDVKATKFDKALRNFMINNVINSNRFNYVTRFINPAEGARIMQSKVLVEFALKDIPEAQLIRYCKTAGITFDADNPDCAKMAKDLADMLNKVRFDNFVKVKQIVKADSEEAKQKERYKAIIRLYLTVLYLIVKSLVKVNMSYSIAFGILERDCEIMNQKYECKDYFIRSEPHSGQVRGYDFAATKITDDFRKDGRLNKRVANSIETNKELYKDDLFYAYRNQVAHLNVIETLPNYVDRIKNVKSYFDLYHCILFLNLMENKAPYLQESINTVKNHVSSNGKTVYENVCEHNTVSKDFLYGLNTPFAYNAARYINLSNREKFIEGYGK

SEQ ID NO: 9 - protein, 930 aa, unidentified
  source 1..930: note = MG103-8 Effector; note = uncultivated organism
MEKNETKNIDIKAKKKSSAKTAGVKSVMKTSADTTVIAVFGKGNEAVLEKRIINNETTDINKSSKISVDAKSEKKRYVITSKSRNAGKSTSAIKGYTDKPDEHSGLDLLGLKDELEQEIFDDNFQDNLRVQIAYNIFDIVKALAPVANNITYTLNSLDRKNGHKNDIIGFTLNYSVAYNQFGAGNDPKVRKKKQDLQNYIANAKNNFIYYDDIFYHDEIISKNDKKTSKNDKKISKNKTVSKLNSEKDIYYSLGLINFLRNSVSHFGDKFLLFKKDYQFMNGDLKNRLDETFRQKVRSVNKNFIANEKNNLWLAANALNIKGDEGLKNLACELYELSVLKSNKNLGFNLKKLRESAISQQIINLPFSEHLLNTFKSKLYKIFDLVIIHHLTQNDNQFIDNFVDALRKSHDDDEKEQLYKQYADELFNNKTLKAQLNKVIKSFSNGDYKKRENVIKINHAWIESVEIDNNAHDFSKLVYFISRFLDGKEINILLTSLISKFQVIDAFNKTVSELVGMNEIKPSDYSEKYGLFNDSGKIAEELNFIKSITKMTTKYENASLESMYKDALLTLGVSKNDVDRLYKQYFDKDSKTKLSAFFKNNILESGRFKYVIKYLNPSHISKIARNKAIAEFVLNRMPDTQIDRYYKSVVNPKVESTPKGNKIHQIADLIKDTKFDDFTGKALSNVKKGDRSRKGDMPEREKKKALLSLYYTIVYIFVKNLVQINSCYLLGYFYLERDKYFFGRKYSSAFGRNTFNVKNNYDALTRLFTGVVQSDVRKLRNNDAQKLKRYIYVSDKNSFSERYDSMYKEFRNSVDHINVISYIGSYVDKITSLDSYFDVYHFILQRTVYSKLVSIASGSQNSRSKDANKSVEYINSLLNISDSSNDFETGENVAPYSRTLLRAANVPMGYTLPRYKNLSYEKLFVRGRENS

SEQ ID NO: 10 - protein, 968 aa, unidentified
  source 1..968: note = MG103-9 Effector; note = uncultivated organism
MKKKLNGNGAIGSASLKKAFQQRQNAQSKAILAQQEEENRKKELREQQAREAAAAAEALRRKELEKNLHPVRERKLSTEKKSSAKAAGLKSTFILSDNELLMTAFGQGNEAIIEKKITDGRVENLNEKPAFTAVPEEKKAFRIDGRVKGAQTDNPLYTADASLRKPDDLIHAKAALEKRYFGQTFDSNLHIQAIYSILDIEKILAIHVNNIVYTLNNFLRQEGEDVDDFIGYVTSGEKTYQQFMESKDNNADELRALFTRLCSARQLRYLSLEIMKPRNPGDNTKEDENTIKLTEEEFYNILYAMGIMRQMLAHGDPYKNIYTLEGYGKNASVEKVLTRLYKERINQLNTDFLSKAGTNLTLLFKAFGITDVEKKGCLVRDYYDFTVRKQYKNLGFSIKLLREHMTADIEEAFILRDKAYDSVRGKLYPFIDFALFRYYHTHNEETNSLVNGLRASFNEVEKDGLYAREAARIWPKVRSVILEHILPEMSGEVIIEAKSNSDKDIKETMLSGAMISTEATDFSKFIYLTTLFINGKEINDLLTTLIHKFECIFSFLAVIQSQHLPSRFVDQFSVFSESDKIAQELRAINSFARMSRPSAKAKEIMYIEAFQVLGMRANSDQLEREAKELLDPKISGERSQKRGIRNFISKNVIASDRFKYLVRYGNVKKLKGIATNRAVVSFVLQDIPDSQIARYYNSVTGEDKDFQPAMRNFLADRLTGFSFEDISDVRQNDQRANQRQQEEKRQKQALVRLYLTVLYLTLKNLVYVNSRYFLAFHCMERDRRLLHPEKWEKIPREKQFDPEFGASVFARDFLAEYPRKTRQMTYLNQNFDNSDEWALRAFRNKAEHLDAVRNADLYLGDIREFHSWFELYHYIMQRRIMDQFTFDSTHKTRDKTRMIISEEELNPKTREYFHLIARYRVYCKDFVKALCVPFAYNLPRFKNLSIDGLFDKNRPGKEKDGEETLPED

SEQ ID NO: 11 - protein, 932 aa, unidentified
  source 1..932: note = MG103-10 Effector; note = uncultivated organism
MKKKENAKQRRDRAKAMRRSTVEAAETKRNEERRLAEEIHIVEEEVRKKQKEEEKRLRAIKAAEKADISLAKKTKTSAKAAGLKSTFVIDDKRLLMTSFGKCNRAIADKYIDDSDIKDAGEEASLRVERKGKNFQVSGRKVKDATADNPLRSKKPAGQDQIFCKDQLEHIYFGTSYVDNIHIQLIYNILDIEKILAVQVNNIVYELNNILREDKAEYDDLIGYMSCDYTFERFKKHNNGKLYEKFRKLIGQPQLGYFGKTFIPYDKAGKKLSSNAMPAFEEKCYNLLSVIGTVRQATAHGSERTRAKLYQFEKEFDGENKGCRIQARRALESLYRERVCSLNSGFLDMSGKDLYILFNAFDAFDNEKKKQIVREYYNFLILKAYKNMGFSIKKLREKMIARCAPELASDDFRTVRQKLYRFVDFAIYRFYLNDENRAYTEEFVASLRASVKASEKEAMYQLEAERLWKELKSIINERITTWMDGERIAKIKKPEIDKHVIDDIAIPESANPFCEMIYLLTIFLDGKEINDMLTQLISRFENIAGFIDVLKGEGMDRAFAPPYGLFDSSDKIAEELRIINSFARMTKEDPSAKPIMYKEAAQVLGFGNEEELEDYVNDMLDKDHKGGMGFRNFIANNVIESSRFKYLVRYGNPKKVRAIAENRKVVDFVLKEIPDPQIQAYYSSCTSGTTGNYDEMRGALATIITGLHFKDFEDVVQGNNASIQQKRDKERKKNIIRLYLTVLYLAQKNLIYVNSRYFLAFHCAERDAMTYDSNRFNKDALWEDRAAFAKAFIAEHPKKKRVQLYLYKNISNSDVWSFRAYRNLAEHLGAIRNMDKYIEDIRGFESYFELYHYLMQRSIIDRFRYEGRTESGAEGKLLEYFELVEKYNTYCKDFVKALNIPFSYNLARYKNLSVNELFDRNNYLPNKEREKDE

SEQ ID NO: 12 - protein, 909 aa, unidentified; identical to SEQ ID NO: 4
  source 1..909: note = uncultivated organism; note = MG103-11 Effector

SEQ ID NO: 13 - protein, 986 aa, unidentified
  source 1..986: note = uncultivated organism; note = MG103-12 Effector
MKKGSSIKEQREREKAQRMSAYKQRDAERKEAVALSENEKKQATAQKASGEDNKAEKKSTAKAAGLKSTFVIKGDELLMTSFGKGNDAIIEKRIRNAVVEDVSENPILSVFPENERYFRIDGRVSGATTDNPLHSAGEEKEKTDLIHAKNALEKRFFGSNFEDNIHIQMIYSILDVEKIMAPHINNIVCQLNNMLRNKGSDNDDLIGYLSLEREKSFEKIKANGSAPVKEGEKLSAAQWKYMLVRRLARNGTMGYYNLEVLRDEREQEEWNRIQEERKQKCEVENREYKAPEEDRNTIKLSYEEFYQTLCLLGEIRQSLAHGNEPSFLYRIEDFDKKFPQSAKVTVQRLYHDRVQELNRKFLKMAKTDLILLFRAFGVQTLEEKKEYVQAYYDFIVRKQYKNLGFSIKKLRESMIEVIPEAMEAGDDKYSTVRNKLYRFLDFAISQHFIKKEEAQENLVGQLRASRTDEEKTEIYVKNAKELWPQIRELVIKHILPQMKVTEVAKVIEENKKGDKDVTEQMLEGRWISDSATFFTTIIYTLTLFLDGKEINDLITQLIHKFESISAFVEVMRYEGMPFSVSGKYDVFNQSREVAAELRVLNNIARMTRPAAHTKPVMFKDALKVLDMQMDNSEIDSYVSTLLDPVLSAAGSHNRGIRNFIANNVIESSRFHYIIRYGNVNKVKGLINNPRVVRFVLKDIPDNQIHRYYRTVTGQDVSFDNSMRRYLADRLTSFSFVELRDVRQSDSGASAADKAEKEQKRAMVSLYLTILYLIIKNLVYINSRYFLAFHCVERDRLLLDEEHWGKMGQANQDDPDYSYKAFARDQLAKYPQKPKVQRYMAQNFANSDDWAIRAYRNKIEHMVAVRNADRYLNDIREFHSWYELYHYTMQREIRAQYEWESARESSRTPGRMIVEKEDLNQKTFEYFDKVEKYGTCCRDFIKALNVPFAYNLPRYKNLSIDGLFDKNRPGKKGEGKKLELDEKTYEN

SEQ ID NO: 14 - protein, 827 aa, unidentified
  source 1..827: note = MG103-13 Effector; note = uncultivated organism
MGKNRIKNHKTAAKAMGLKSTFILDDENVFITSFGKGFDADPEKTVQRDEIINHKNTFSVQKKSSADEKVQKLEMHGRTIVKPVSMYKDGTTKNCETTNKTVVDIPTKDSNMLHAKDIIEKMYFGETFDDNIHIQIAYNIMDIKKIFSLYANIVVNSVNNINRDKLDEDFLGFIFATQNTYEASKLAYDLSKTGALEVVKNEKERFLINFKKLNKACNKINESKKYEYPEIKGLMDEVNTIEKEIPLPIKIINVLKKYNIKPKYFDKIVKNFSNFLCFSDYMKQNAVYFPNLFYTNERFDMKKAYEIFRILGNLRHGSFHENDSSKSWILTIDKNTDERLKNTVNNIFDTKLNKINSKFVKTSKKNLLILQMLYPNNKDIVQQYYDFAVRKAYKNLGFSIKDVRETILTFDDAKHLNEQKFDSVRGKLFTLFDFVIYDYFINNTEISQRLVNELRATMSEDEKRSIYVKYANDVWSQIKNFILNTVVKKLNSDEINNLNGNISDSKLLDEIQKPEDISLFAKTIYCISMFLDGKEINMFLSSLINKFENIASLVETLSYNKIPLNLNEDYFIFENSSKYANDLAFVKSIAKMCKTKKAIKQGDEKAKSSVYYDSAALFGEFDKKRVDELYHLGDKNATSSQKALRNFMINNVINSNRFSYVTRFINPKTARDIMQSEGLVKYILRQLPESQLIRYCNTAEISYNANEPDFEAIIDELTKMLVKVELSQFLNVKQQVIEGSKQEKEKEKLKAIIGLYLTVLYLIVKSIVRINSSYTVAIGILERDTAMFFPDDFKKIKDFAISRALTDRFMDAGYLKNKNVLENIRLS

SEQ ID NO: 15 - protein, 939 aa, unidentified; identical to SEQ ID NO: 3
  source 1..939: note = uncultivated organism; note = MG103-14 Effector

SEQ ID NO: 16 - RNA, 864 nt, synthetic construct
  source 1..864: mol_type = other RNA; note = cis-cleavage target
gggagaccacaacggtttccctctagaaataattttgtttaactttaagaaggagatataccatggagcttttcactggcgttgttcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatctaactcgagccttaggagatccggctgctaacaaagcccgaaaggaagctgagttggctgctgccaccgctgagcaataactagcataaccccttggggcctctaaacgggtcttgaggggtttttt

SEQ ID NO: 17 - RNA, 101 nt, synthetic construct
  source 1..101: mol_type = other RNA; note = target for trans-cleavage assay
gacattaacctataaaaataggcgtcgaggccctttcgtcttccgtgagccaccacgtcgcaagcctcgactttaagccagccccgacacccgccaacacc

SEQ ID NO: 18 - DNA, 300 bp, synthetic construct
  source 1..300: mol_type = other DNA; note = 103-2_30_REV_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactatagggtcaatacccttctaaaattagaagggtctaaaactggagatatcttgaaccttgcatccccggaggtcaatacccttctaaaattagaagggtctaaaacaggctaggtggaggctcagtg

SEQ ID NO: 19 - DNA, 300 bp, synthetic construct
  source 1..300: mol_type = other DNA; note = 103-2_MA_30_REV
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactatagggtcaatacccttctaaaattagaagggtctaaaacccgtccagctcgaccaggatgggaacaacgggtcaatacccttctaaaattagaagggtctaaaacaggctaggtggaggctcagtg

SEQ ID NO: 20 - RNA, 124 nt, synthetic construct
  source 1..124: mol_type = other RNA; note = 103-2_MA_30_REV
gggtcaatacccttctaaaattagaagggtctaaaacccgtccagctcgaccaggatgggaacaacgggtcaatacccttctaaaattagaagggtctaaaacaggctaggtggaggctcagtg

SEQ ID NO: 21 - RNA, 36 nt, synthetic construct
  source 1..36: mol_type = other RNA; note = 103-2 repeat
ggtcaatacccttctaaaattagaagggtctaaaac

SEQ ID NO: 22 - DNA, 300 bp, synthetic construct
  source 1..300: mol_type = other DNA; note = 103-3_MA_30_REV_U67
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactatagctattacactacaccccttttacaggggtctaaaacgtcgaggcttgcgacgtggtggctcacggactattacactacaccccttttacaggggtctaaaacaggctaggtggaggctcagtg

SEQ ID NO: 23 - DNA, 300 bp, synthetic construct
  source 1..300: mol_type = other DNA; note = 103-3_30_REV_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactatagctattacactacaccccttttacaggggtctaaaactggagatatcttgaaccttgcatccccggactattacactacaccccttttacaggggtctaaaacaggctaggtggaggctcagtg

SEQ ID NO: 24 - RNA, 300 nt, synthetic construct
  source 1..300: mol_type = other RNA; note = 103-3_MA_30_REV
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactatagctattacactacaccccttttacaggggtctaaaacccgtccagctcgaccaggatgggaacaacgctattacactacaccccttttacaggggtctaaaacaggctaggtggaggctcagtg

SEQ ID NO: 25 - RNA, 124 nt, synthetic construct
  source 1..124: mol_type = other RNA; note = 103-3_MA_30_REV
gctattacactacaccccttttacaggggtctaaaacccgtccagctcgaccaggatgggaacaacgctattacactacaccccttttacaggggtctaaaacaggctaggtggaggctcagtg

SEQ ID NO: 26 - RNA, 36 nt, synthetic construct
  source 1..36: mol_type = other RNA; note = 103-3 repeat
ctattacactacaccccttttacaggggtctaaaac

SEQ ID NO: 27 - DNA, 300 bp, synthetic construct
  source 1..300: mol_type = other DNA; note = 103-4_30_REV_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggtactaataccctacgaaacagtagggttctgagactggagatatcttgaaccttgcatccccggagtactaataccctacgaaacagtagggttctgagacaggctaggtggaggctcagtg

SEQ ID NO: 28 - DNA, 300 bp, synthetic construct
  source 1..300: mol_type = other DNA; note = 103-4_MA_30_REV
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggtactaataccctacgaaacagtagggttctgagacccgtccagctcgaccaggatgggaacaacggtactaataccctacgaaacagtagggttctgagacaggctaggtggaggctcagtg

SEQ ID NO: 29 - RNA, 124 nt, synthetic construct
  source 1..124: mol_type = other RNA; note = 103-4_MA_30_REV
ggtactaataccctacgaaacagtagggttctgagacccgtccagctcgaccaggatgggaacaacggtactaataccctacgaaacagtagggttctgagacaggctaggtggaggctcagtg

SEQ ID NO: 30 - RNA, 36 nt, synthetic construct
  source 1..36: mol_type = other RNA; note = 103-4 repeat
gtactaataccctacgaaacagtagggttctgagac

SEQ ID NO: 31 - DNA, 300 bp, synthetic construct
  source 1..300: mol_type = other DNA; note = 103-6_MA_30_REV_U67
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacacccttacgaactcgtaggggtctgaaacgtcgaggcttgcgacgtggtggctcacggagaactacacccttacgaactcgtaggggtctgaaacaggctaggtggaggctcagtg

SEQ ID NO: 32 - DNA, 300 bp, synthetic construct
  source 1..300: mol_type = other DNA; note = 103-6_30_REV_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacacccttacgaactcgtaggggtctgaaactggagatatcttgaaccttgcatccccggagaactacacccttacgaactcgtaggggtctgaaacaggctaggtggaggctcagtg

SEQ ID NO: 33 - DNA, 300 bp, synthetic construct
  source 1..300: mol_type = other DNA; note = 103-6_MA_30_REV
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacacccttacgaactcgtaggggtctgaaacccgtccagctcgaccaggatgggaacaacggaactacacccttacgaactcgtaggggtctgaaacaggctaggtggaggctcagtg

SEQ ID NO: 34 - RNA, 124 nt, synthetic construct
  source 1..124: mol_type = other RNA; note = 103-6_MA_30_REV
ggaactacacccttacgaactcgtaggggtctgaaacccgtccagctcgaccaggatgggaacaacggaactacacccttacgaactcgtaggggtctgaaacaggctaggtggaggctcagtg

SEQ ID NO: 35 - RNA, 36 nt, synthetic construct
  source 1..36: mol_type = other RNA; note = 103-6 repeat
gaactacacccttacgaactcgtaggggtctgaaac

SEQ ID NO: 36 - RNA, 57 nt, synthetic construct
  source 1..57: mol_type = other RNA; note = Processed crRNA 21 nt spacer
gaactacacccttacgaactcgtaggggtctgaaacnnnnnnnnnnnnnnnnnnnnn

SEQ ID NO: 37 - RNA, 62 nt, synthetic construct
  source 1..62: mol_type = other RNA; note = Processed crRNA 26 nt spacer
gaactacacccttacgaactcgtaggggtctgaaacnnnnnnnnnnnnnnnnnnnnnnnnnn

SEQ ID NO: 38 - DNA, 300 bp, synthetic construct
  source 1..300: mol_type = other DNA; note = 103-9_30_FWD_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacacccctacggaatggtaggggtctaaaactggagatatcttgaaccttgcatccccggagaactacacccctacggaatggtaggggtctaaaacaggctaggtggaggctcagtg

SEQ ID NO: 39 - DNA, 300 bp, synthetic construct
  source 1..300: mol_type = other DNA; note = 103-9_MA_30_FWD
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacacccctacggaatggtaggggtctaaaacccgtccagctcgaccaggatgggaacaacggaactacacccctacggaatggtaggggtctaaaacaggctaggtggaggctcagtg

SEQ ID NO: 40 - RNA, 124 nt, synthetic construct
  source 1..124: mol_type = other RNA; note = 103-9_MA_30_FWD
ggaactacacccctacggaatggtaggggtctaaaacccgtccagctcgaccaggatgggaacaacggaactacacccctacggaatggtaggggtctaaaacaggctaggtggaggctcagtg

SEQ ID NO: 41 - RNA, 36 nt, synthetic construct
  source 1..36: mol_type = other RNA; note = 103-9 repeat
gaactacacccctacggaatggtaggggtctaaaac

SEQ ID NO: 42 - DNA, 300 bp, synthetic construct
  source 1..300: mol_type = other DNA; note = 103-10_MA_30_FWD_U67
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacactcctgactgttgtcgggagtctaaaacgtcgaggcttgcgacgtggtggctcacggagaactacactcctgactgttgtcgggagtctaaaacaggctaggtggaggctcagtg

SEQ ID NO: 43 - DNA, 300 bp, synthetic construct
  source 1..300: mol_type = other DNA; note = 103-10_30_FWD_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacactcctgactgttgtcgggagtctaaaactggagatatcttgaaccttgcatccccggagaactacactcctgactgttgtcgggagtctaaaacaggctaggtggaggctcagtg

SEQ ID NO: 44 - DNA, 300 bp, synthetic construct
  source 1..300: mol_type = other DNA; note = 103-10_MA_30_FWD
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacactcctgactgttgtcgggagtctaaaacccgtccagctcgaccaggatgggaacaacggaactacactcctgactgttgtcgggagtctaaaacaggctaggtggaggctcagtg

SEQ ID NO: 45 - RNA, 124 nt, synthetic construct
  source 1..124: mol_type = other RNA; note = 103-10_MA_30_FWD
ggaactacactcctgactgttgtcgggagtctaaaacccgtccagctcgaccaggatgggaacaacggaactacactcctgactgttgtcgggagtctaaaacaggctaggtggaggctcagtg

SEQ ID NO: 46 - RNA, 36 nt, synthetic construct
  source 1..36: mol_type = other RNA; note = 103-10 repeat
gaactacactcctgactgttgtcgggagtctaaaac

SEQ ID NO: 47 - DNA, 300 bp, synthetic construct; identical to SEQ ID NO: 18
  source 1..300: mol_type = other DNA; note = 103-11_30_FWD_NT_U40

SEQ ID NO: 48 - DNA, 300 bp, synthetic construct; identical to SEQ ID NO: 19
  source 1..300: mol_type = other DNA; note = 103-11_MA_30_FWD

SEQ ID NO: 49 - DNA, 124 bp, synthetic construct
  source 1..124: mol_type = other DNA; note = 103-11_MA_30_FWD
gggtcaatacccttctaaaattagaagggtctaaaacccgtccagctcgaccaggatgggaacaacgggtcaatacccttctaaaattagaagggtctaaaacaggctaggtggaggctcagtg

SEQ ID NO: 50 - RNA, 36 nt, synthetic construct; identical to SEQ ID NO: 21
  source 1..36: mol_type = other RNA; note = 103-11 repeat

SEQ ID NO: 51 - DNA, 300 bp, synthetic construct
  source 1..300: mol_type = other DNA; note = 103-12_30_FWD_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacacccttaccgattggtaagggtctgaaactggagatatcttgaaccttgcatccccggagaactacacccttaccgattggtaagggtctgaaacaggctaggtggaggctcagtg

SEQ ID NO: 52 - DNA, 300 bp, synthetic construct
  source 1..300: mol_type = other DNA; note = 103-12_MA_30_FWD
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggaactacacccttaccgattggtaagggtctgaaacccgtccagctcgaccaggatgggaacaacggaactacacccttaccgattggtaagggtctgaaacaggctaggtggaggctcagtg

SEQ ID NO: 53 - RNA, 124 nt, synthetic construct
  source 1..124: mol_type = other RNA; note = 103-12_MA_30_FWD
ggaactacacccttaccgattggtaagggtctgaaacccgtccagctcgaccaggatgggaacaacggaactacacccttaccgattggtaagggtctgaaacaggctaggtggaggctcagtg

SEQ ID NO: 54 - RNA, 36 nt, synthetic construct
  source 1..36: mol_type = other RNA; note = 103-12 repeat
gaactacacccttaccgattggtaagggtctgaaac

SEQ ID NO: 55 - RNA, 57 nt, synthetic construct
  source 1..57: mol_type = other RNA; note = Processed crRNA 21 nt spacer
gaactacacccttaccgattggtaagggtctgaaacnnnnnnnnnnnnnnnnnnnnn

SEQ ID NO: 56 - DNA, 300 bp, synthetic construct
  source 1..300: mol_type = other DNA; note = 105-1_MA_30_REV_U67
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggttgtaacagcctttagtttgtaaggtaaaaacaacgtcgaggcttgcgacgtggtggctcacggagttgtaacagcctttagtttgtaaggtaaaaacaacaggctaggtggaggctcagtg

SEQ ID NO: 57 - DNA, 300 bp, synthetic construct
  source 1..300: mol_type = other DNA; note = 105-1_MA_30_REV_NT_U40
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggttgtaacagcctttagtttgtaaggtaaaaacaactggagatatcttgaaccttgcatccccggagttgtaacagcctttagtttgtaaggtaaaaacaacaggctaggtggaggctcagtg

SEQ ID NO: 58 - DNA, 300 bp, synthetic construct
  source 1..300: mol_type = other DNA; note = 105-1_MA_30_REV
atacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttaatacgactcactataggttgtaacagcctttagtttgtaaggtaaaaacaacccgtccagctcgaccaggatgggaacaacggttgtaacagcctttagtttgtaaggtaaaaacaacaggctaggtggaggctcagtg

SEQ ID NO: 59 - RNA, 124 nt, synthetic construct
  source 1..124: mol_type = other RNA; note = 105-1_MA_30_REV
ggttgtaacagcctttagtttgtaaggtaaaaacaacccgtccagctcgaccaggatgggaacaacggttgtaacagcctttagtttgtaaggtaaaaacaacaggctaggtggaggctcagtg

SEQ ID NO: 60 - RNA, 36 nt, synthetic construct
  source 1..36: mol_type = other RNA; note = 105-1 repeat
gttgtaacagcctttagtttgtaaggtaaaaacaac

SEQ ID NO: 61 - RNA, 56 nt, synthetic construct
  source 1..56: mol_type = other RNA; note = Processed crRNA 20 nt spacer
nnnnnnnnnnnnnnnnnnnngttgtaacagcctttagtttgtaaggtaaaaacaac

SEQ ID NO: 62 - protein, 894 aa, unidentified
  source 1..894: note = uncultivated organism; note = MG103-15 effector
MEHIPETKTLKELKPKSKKAKAAGLKSTFVIGNDLLMTAFGKGNDAIPEKEIKNGEIHSLNSETKFDVKSLENGFNVKGRGITTNTVNPLVSVKTVGEDLIGCKDALEEKYFGKTYCDNIHIQLIYNILDIEKIMAIHINNIIYELDNLQRREDVEYGDFIGYMSSKNTYDVFCNYEKHFKKDSKIYDNLKKQNWQFDNYKGNKRLAYFGTTFYKIDQKRSTKSKTKYVIDPEKMYYLFAILGDIRQFCAHSQKNTEYSLYDMNTFLNDEAKAVLDELYSSEIDTLNNGFINHNGKNISLLCEILNVTNTQNKVVLIKDFYEYIVKKSFKNIGFSVKKLREYMIEQNIIELKDKRYDSVRSKLYTLLDYIIYHYYDKDKIEKNVDALRTCLTDKEKEQFYIKEANTLWLGINNKVATLKRKMNGNYIRSYEVSPNIQMLIKNTVNEVALSNTTDYFCKVIYLMTLFLDGKEINDLLTTLVSKFDNIASFVNVIKDRDIECNFKHNYKLFERSDKICQSLREINSFARMVKMEDDAKRVLYVDAAEILGTDMNEEELTNYLDEKVLSKKLGRRNHGFRNFLANNVIESSRFKYLVRYANPKKVRAVAENQNVIKFVLKRIPETQIDRYCDSCFNKIPQDYDTKIQNLTNLITGISFKNFEKVNQSATVGTFEGKDKQQKQAVISLYLTVLYHLTKNLVYVNSRYFLAFHCLERDAQLHKNTAGYDRKNWCNLTRQLSDKRLRDIDAWYGTLSEPNRNQKKRKWKAEQNCGYLAENISHIDDKTITAYRNNVEHLSPIRNINRFIGDTAKFNTYFELYHYLIQRTVAETSEKFLKRPLLPKVQEYCDFVLKNKTYCKDFVKALNSPFGYNFPRFKNLSIDALFDMHNPPLAENEEK

SEQ ID NO: 63 - protein, 783 aa, unidentified
  source 1..783: note = uncultivated organism; note = MG103-16 effector
MKKKKQSKKSGIKIIANKDNKIYFFEEDKHLNDDTKNYLKLEDNNQKIKELENNIKNLKFIDENTNKNISLTKDIKVSIYENKYTLKKNNIINDALIKKYKAENSNTNITIQIIHTNEDIKKISSIYINDVVFSINNTMSEDKQDIVSKITKKFFYDEKTQKIRVEQKDGSIEDYNNDDLKNLFKNLRNHKFFFGFSDTKEKFLFEHLKSISLFRNYISHDNLIANSNEIISKELNKYIDKNGKDFLENNKFNVSLISKLYKNNKEDELKKYYEYVINSKDKSLGISIKKLVEYSLIKYPPNLDAEKIKKYHTVLKYHLFNIINKNESLKNKYISKLRKSDFEEKEKIYNDLLEELQSKPFNDVEQICSNIDFRNKQKEKIDIKNIILNEHKDNWLPFTLYVYSLTKFLTIKETNELISNLISKLESMNDLIIASKKLGINIKDNLDLTLFIVNFDDLILEINQLRIINSIKKFSAPKHSDSSMDYYKKGLEIFKNSNLNLDELLKEKTKNEILKQKKIKHPIKNFVRNNVVKTKFFNYIIRYADPKKISELLYDNPKLVKFLLSNLSESQLNKFKNDKNLEEIISKITLETINNNLKNGNTEYTKYINLYLTCLYFLIKNLNRINAYYYLAFSFFVRDHQLLFNEDPFKTEDYMKLTNMQNKSIKDIKNINKSIESFNKINKKMFKEYRNEIAHFGIINILSETKFPEDFDFKNYFKIYHYCLQKKILSPNNEYLSKITKDIDKYNKYSKNFLFSINTPLMYNYSRYKNISDENIFMRLYDK

SEQ ID NO: 64 - protein, 949 aa, unidentified
  source 1..949: note = MG103-17 effector; note = uncultivated organism
MEKSNGIHNREEEIKELKRLQKKQNRKTSAKAAGLKSVLLTDKKTVLTSFGKGNDSIVEKIIEEKKIKDVADTKHYDARIIETKTRNTNKEELGIDGRLIKDAKSDLPKSVRKDQLGAKDKLEEIFFGEQFPQDNIRIQMLYNVLDIEKEYSRHIVNALYGINHLNRNIQGGRNEKNDYVGNIFASDKMSKIKSEDAKQKEGNKIPHNGTKYDRFLNYYKGAIDETDGKRKDGARDYLIYYGEAFDDAITELQSQRPDYQIAEELVYDRLCILSLLRQGVAHGEGGFETDIVLAPDAVLKSEKHSRLQSLLKKDYLKRVKGIDDDFIKNNKNRNLPIIFNIYNAVSEKEKKTLSIEFYKYVVLKENKNMGFSLKKLREMLLLKPEFVFMKNEEYDTARGKMYSIVDFVIYKYYAMQNANAKEIGIVERLRGTKSKSEKEAIYKEEAAHLAENIKGTLSTKNMSSFKSNEIKDIKGDDTVKENWLNGEKISDKEVSNFTLLANFMSMFLDGKEINEFLSILINKLESIAALKEVINNETISPKKDNKKEKTYSGFEALAAALATNDISIKEEFEWLKNAGEIATEIRILKNVARMKFADDLNFKRPMYCDAVNVLSNNKSAEALSEFIDEEMLSKNVKEKNLRNFISNNVIKSRRFKYLMRYSNPSIVRKLMQDENLIEFVLKRIDNNNPEQLEKYMNSIHEAGEDRVAVLTRKLLALRFEDLFDIKQKVRTGSNEAVKKETYKAVLGLYLTVAYIAIKNLVNINSRYVMAFSALERDSSIFLSRYDKEQPLKLLDKKLGEGAINRRYAQIFKENERNVIENAFRIYRNNVAHLTVVEKLPNYLGKGEGQKREILSYFELYHFILQNLINSECKSREKHPGSNLVYSNTQLANEIQKISNYRSYNKDALNILNTPFGYSLARYKALTVEALFDKNYPEERLKMLGECDND

SEQ ID NO: 65 - protein, 1004 aa, unidentified
  source 1..1004: note = uncultivated organism; note = MG103-18 effector
MAEKRFSKAKAAGLKSSLVTDNSVYMTTFGKHNEAILEKKIEDGRITNINNPEKLAVRIINNPKMSVSGLKVMDAVADNPLSKKAKNKTGDSDKLRSSRYIVGMDQLRIKEKLEEKYFGEVFDNENIHVQIAYNILDIKKILSVYINNIIYSVNNLRRLEVNGMEREDDFIGFLYTDNKYQSLADNCARCRFPEKFGSDLSKCSCFKTKWDESLCEKAKKYNESSKVCDKCRHASKCSFFSRSNKVSARPVWWSPENCNTFKELAHNCRICESRKECRYNLSGMKTTSNNWNFDKCGSAYSFGSYMNSVKPYIGYFRDVFYSGRFETIRGKNGKTEKKELPRDYKDIYNILRIVGYMRQSVMHEAVKTADFLFAPVTDPEIKELIDRLYSEKVTYANRSFLKNNEKNVTLLFDYYNCASNSDKVKLIQDFYDFIVCQEQKNLGFNLRTIRENLLEDDTLKYIKGNSYDSVRSKLYTLLDFVLFKYYKGTPLQEEIVENLRACTSQEDKDIVYEKQAKEENLLREAKRFSKVIMPQMKGDIIKGTDKSRIDSEWISGVKLETKASDFTKILFVLTMFLDGKEINELLSGLQNKLSNISSFTKVLEEMDKSCSFVEKYKMFGKSETISRELRTLKSFVRMQREIPKHSRGAYIDAVDILGIDRANTTLSNMSDDDTAEELVEKFFIGGKDNNVRNFIINNVLESRRFRYLVRYNNPKSTRQLAKNKDVVKFVLNGIPEAQINRYYTTVTGDDATIVSLERQIDELSNRISRIDFNHFTSVKQKPNTSEENIRKERMKAIIGLYLTVMYLVIKNLMRVNARYSIAVNCFERDTQYYNVFFKNQEVISGKLDPKELADRFADNGWSRDNWRDKDGRQHSVKEYGEKLWGASFGFYRNAIAHHNVIAKVGYYLSAEAGDARITKIKSYYQLYHSVMQRVLDYELRNPSNPDEDAKRRCDYFRQCVSLARENGSYSKELVKVLNYPYSYNLARYKNLVNEKTFEADRSKP

SEQ ID NO: 66 - protein, 1082 aa, unidentified
  source 1..1082: note = uncultivated organism; note = MG103-19 effector
MKKNKRQSADQRRQEQKAKRMQQLQEKGCKENVVEKQKEGFTDMAQEARKQTQTEKEKIHVKALGLKSILVMDDRIAVTSFADTKLPDRKGAAGEPRQIKTSNLEKVTSLEGDPIESVKTHERMFHVDIDREIIDLRAKGNEDRRVSLENPAKGNVGEDYIGVKAQLEESVFGRSFPMDNLHVQIAYNIFDIKKILGTYVNNIIYIFYNLNRTGEKKEEEEKEKAGAEGKQSEDLIGTLYTDKSLSEQEEWAREDPEKQETRQKALNKVKRLLKDTSPYQVYFGSLFKQVKSYQGEKSDRFLTKEELSEKKEKYRKEQEEEALSYNYNILRLLSLARQLCMHAQTGSAGIASLYNLDKELKEKNPELLALLDEKYKAGVEKLNRGFTKNAENNLYMLQKVYPDTPLDKLAEDYYYLTIRKEALNIGIDLRRLRSYMIEKYFPEAEDQRHDTYRSKIHTIFDFILIDYIRGNDGAEISVRQRMIGELRLAQKDDESKERIYRKYAGIYWEKVQNRWRVGFGAVIRQADSKFSGKAQVGKEFYEKSCVISEKNTDAFVKFLYFICKFLDGKEINELLCAMINKFDNIADLTDAGAACGKPIVFGENYMIFSRSRDIGEQLRIAKNLAAMEFTGVKKQKKKADKNELVYPEILYRDALTLLGYSFQTYKDGSSPEKLSSLSVEERKKWYTPEYMEFRRVFYETNKTDEYGNVKHKNGKPEINHQTRNFICNNVLMSKWFFYVVKYNRPWKCRTMMKNESLLRFVLRDIPDTQIDRYYETVMGRRGGVSQEDKRAELVNMLTKFSGKSILKTVPEISEREYKRQDPTGRKEKLKGIVRLYLTVAYLITKSMVKVNTRFSIAFATLERDYFMKIAAKDTSQLNGTEYLEMTKMAFEEDRAVFDKNTARWKELNGKIKAEAPQSEARKQLRKEMDKLRKEKTHYSFHWWSCLKTNMEEAEDLKFISKESNTYRKYRNAVAHLNVIDKMNIYTEGARVESYYGFYCYCMQMILLGERTEKGVAEKMAPVRQTGNYSRNLMWILNLPFAYNLARYKNLSNEALFYDQPDENEPGKKEALSSSPKSEKAEN

SEQ ID NO: 67 - protein, 977 aa, unidentified
  source 1..977: note = MG103-20 effector; note = uncultivated organism
MNDNTMKSKAKAAGVKSGIVLSRETVIMTAFGKGNAAILEKKIDRATIAPLSSPENFGLSLKNAASGNRKERRAGDPEKQAQREAYWKDHPEAKAKYEAAHAAPPATGDAVLAPASTADAYAVLSKRPFAPADALRDNPLKANPRDQLRAKDKLESLLFGETFADTLRVQIAYSVLDVTKIFTPHIANLVYVLNNLARDEKLSADDVIGGLYIDFEDEKKKPSSKCWEQFLTWKKAAQPAFGYFGDAFPERAKDDRRASEAQKQEKEKRYVDAVWCSVALVSALRASVTHGARTNVIFQPERLHPQILAPVSSVYSQRIDKLNADFIKLNATSNLPLLFHYFDAGTDEKQADMTRAYYDFVVRKASKNMGFSLRTVREEMLRLDADGQRLASDDYSTIRHKLYSLLDFIVYRHFTGLTQGQTPDYIISQLRAATDDAQKQRVYASAAKAAWAKLSAPVLAKLVPRIDRIKSEQGADALSAAEKERLAAALAPVRITADNTSPFSQMVYFVSRFLDGKEINDLLTTLIHAFENIQSFRDVERDLVARGELSGVAAFTKGYRLFERSGAIAAELRVVNSFARMSCESPDIKLPQYLDAARVLGIEAIPEGETEADYVSRVLKLGEKSTRESKVDKSFRNFIAKNIVESARFGYLVRYANPQTVKALATPNLLRFVLGRIWENDAARLKEHAGQRGQKSLIARYTEAMGIPADLPKDRQLDELVERLRSISFETFMNVKQKPGKDENEIKEKERLLALARLYLIALYHIVKNLVYVNSRYLIALQRLEADSALHGTGTFGSNGRLADGRHFTDLTRKFADNRWLNAKMCRLLPANMAAGKYDDWLFVVYRNAIAHGGVLARVSEVCETMGRIPSWFALYHYVIQSACLPAALTGYLENWDDKKGKPEDKAALASALGSYSADNGPLEKTLANGNFSKDVLWSLNAPFGYNLARYKNLSMEALFDRNEAPPSDAPESEQSV

SEQ ID NO: 68 - protein, 932 aa, unidentified
  source 1..932: note = uncultivated organism; note = MG103-21 effector
MSKKSIAKAIGLKSTFKIKDDDYLMTSFGKGNKAQEEKYIENFSVKDLHDTFEAAVDNKSIASIKKEKIGETRVQLPDKDRNQLQAKSVIEKIFFGQAFNDNIHIQIAYNVMDIKKLFSVYANNIIYTVNNLLRMDDDKDFIGTLGTMNNLEAAKIAYAIINSKLITKDKGKSDYYFIDMNVWFSEKDKSLSALNRIVKKNSVKSLKCFKNISNCIVYQLGFTEFKYIKSAAESYEPIKNMGLDKCANYFSDVFADKDGNINLDNIYECLRLLGTMRQTAFHSDDFKDEANRISNTYTLFEIDDKATKETRTVLNAITKNKIETINNNFVNTSTVNLNILFSAYPHMDKKELIKNYYDFAIRKAYKNIGFSVKTLREVMLAEEAELKKLDEKEYDSVRQKLYSLFDFVIYTYYLNNIDASLEFIDLLRQLTVAKDEGISLEEKKLILYKSEAKELWNNIKTIVMNNVIPGVDKCKSSGGIKSLSAEIDDNLKSEILSAIRNSSKLSYFSEAIYCICAFLDGKEINMFLDSMINSFDNILSLIEIAKYANIDVKFSDNYGFFNNCEKYADELRFVKSVARMNKGKKATKDSNVHIKSFQYYDAASIFGETDKNRVTTFFKLDERSTDHTLRNFFINNVINSNRYNYVVRFINPTNARAIMKNEALVSYAISDIDDKQIIKYCESTQISYNAKNPDLAEMRVTLVEKLLKVNFETFAKVSNNSEEAVEKERLKSLVGLYLTIIYLITKSLVRINTSYSIAFSIFERDCIIMDMKAKSTPDENSYLIENISFNNKESNYFGLTEYFIKNNRVNDRVRNLALRNQNHYNNDTFRYFRNNVEHLKVLAKIPVYVSDIVKVDSYFDLYHYLMLRTLDEGYKGNSSIKNVSTSSEIDKVLGYQSAWKDFLYGILAPFAYSPARYINLACKNKFIEGYGK

SEQ ID NO: 69 - protein, 920 aa, unidentified
  source 1..920: note = MG103-22 effector; note = uncultivated organism
MASRKTIAKADGLKSTFYLEPEHFYMTSFGKGMQAKPEKDIQDGTVTDLNNTFTASVPEGCNWISVSGKAGKCQVAEPKLQDQLHAKGTIESHYFGREYSDNIHIQLAYNIMDIQKILAAYSGIVIYTVNRMNRNSDDKDFLGEFKSQNSYDMTRTISKAYELRLLTKHRSGKGFFINGNVWRENQNGELKWLNRQVNIYKQKNFPDQHWNYAIISDFLCSRGIHNMKILKKTYDCWNDFYTFCKSAKDSAYYFGNTFYDEKGNFDENKTFTLLRLLGTFRQDFIHGEQIGGSSLFRSAEEWPADITALLNSLLDGRIENLNKNFCSQNRVNIRILEKIYPDISTKQLVKEYYDFLIRKSFKNIGFSVRTLRETILVQEEEAHALTEKSYDSVRSKLYSLIDFVIYKFYLDREKDIEEMVSALRLSISDEDKERVYQAEAKRLWSYIQEPVMKNIVPAMTSDSIRALSKGKAEAVEDIEDYMEKPGDLSAFSKAIYAFSLFLDGKEINMLLSDLVSKFENIASFLDTLRFLNMDCPFTDNFVFFNDSAKIAGDLRFIQSIARMNKGKKAKKNTTVTVKKCQYADSAAVLGETDESKINRLFNLKSSDAETPSGDNSLRNFIINNVINNNRFTYVVRFLKPADAREIMQCRPLIAYTLQKLPQSQIERYCRAVSVPYAEDECEKAVNELTKLLINVKLDNFVSVKQKASAGSEDAVQKEKYKAVISLYLTVLYLIVKTLVRINSRYMIAFGILERDLALYRIKTGDESFLISDKIKSETVNARPERLTLYFRENYKLNPRVAVSCETNAENFSKNVFKVYRNKVAHLQTVTLLPAYIKEVGIRKVKSMFDVYHCVLLYGISETDFRDETLNAPLKDNLEKCKEYCTVCKDFLYALNVPFAYNAARYINLSNREKFMESYGK

SEQ ID NO: 70 - protein, 920 aa, unidentified
  source 1..920: note = uncultivated organism; note = MG103-23 effector
MATRKTIAKADGLKSTFYLEPEHLFMTSFGKGMHAKSEKDIQDRTVTNLNNTFTASVPVDSNWISINGKAGQCQVAEPKLQDQLHAKEIIESIYFGKEYSDNIHIQLAYNIMDIQKILAAYSGIVIYTVNRMNRDSDDKDFLGEFKTQNSYDMTRTISKAYKLRLLDKNRNGKGFFINGNVWRDNRNGELKWLNRQVNIYKQKKFPDQHWNYAIISDFLCSRGIHNMKILKKTYDCWNDFCTFCKSAKDSAYYFGNTFCDEKGNFDERKTFTLLRLLGTFRQDFIHGEQLGGSSLFRPAEKWPDDVTAMLNSLLDGRIENLNKNFCSQNRVNIRILEKIYPDISTKQLVREYYNFLIRKSFKNIGFSVRTLRETILAQEEEAHALTEKSYDSVRGKLYSLMDFVIYKFYLDREKDIEEMVSKLRFSISDEDKERVYQAEAERLWLYIQESIMKNIVPAMTSDSIKSLSKGKADAIENIEDYMEKPGDLSAFSKAIYAFSLFLDGKEINMLLSDLVSKFENIASFLDTLRFLNMDCSFTDNFVFFKDSAEIAGDLRFIQSIARMNKGKKAKKNTEVTVKKCQYKDSAAVLGETDESKINRLYNLKSSDSETPSGDNSLRNFIINNVINNNRFTYVVRFLKPADAREIMQCRPLIAYTLQKLPQSQIERYCRAVSVPYAEDECEKAVNELTKLLINVKLDNFVSVKQKASSGSKEAVQKEKYKAVISLYLTVLYLIVKTLVRINSRYMIAFGILERDLALYRIKTGDESFRISDKVKSETVNARPERLTLYFRENYKLNPRVAVSCETNAGSFSKNVFKVYRNKVAHLQTVTLLPAYIKEVGIGKVKSMFDVYHCVLLYGISETDFHDETLNAALKDNLEKCKEYCTACKDFIYALNIPFAYNAARYINLSNREKFIESYGK

SEQ ID NO: 71 - protein, 920 aa, unidentified
  source 1..920: note = uncultivated organism; note = MG103-24 effector
MATRKTIAKADGLKSTFYLEPEHLFMTSFGKGMHAKSEKDIQDRTVTNLNNTFTASVPVDSNWISINGKAGQCQVAEPKLQDQLHAKEIIESIYFGKEYSDNIHIQLAYNIMDIQKILAAYSGIVIYTVNRMNRDSDDKDFLGEFKTQNSYDMTRTISKAYKLRLLDKNRNGKGFFINGNVWRDNRNGELKWLNRQVNIYKQKKFPDQHWNYAIISDFLCSRGIHNMKILKKTYDCWNDFCTFCKSAKDSAYYFGNTFCDEKGNFDERKTFTLLRLLGTFRQDFIHGEQLGGSSLFRPAEKWPDDVTAMLNSLLDGRIENLNKNFCSQNRVNIRILEKIYPDISTKQLVREYYNFLIRKSFKNIGFSVRTLRETILAQEEEAHALTEKSYDSVRGKLYSLMDFVIYKFYLDREKDIEEMVSKLRFSISDEDKERVYQAEAERLWLYIQESIMKNIVPAMTSDSIKSLSKGKADAIENIEDYMEKPGDLSAFSKAIYAFSLFLDGKEINMLLSDLVSKFENIASFLDTLRFLNMDCSFTDNFVFFKDSAEIAGDLRFIQSIARMNKGKKAKKNTEVTVKKCQYKDSAAVLGETDESKINRLFNLKSSDAETPSGDNSLRNFIINNVINNNRFTYVVRFLKPADAREIMQCRPLIAYTLQKLPQSQIERYCRAVSVPYAEDECEKAVNELTKLLINVKLDNFVSVKQKASSGSKEAVQKEKYKAVISLYLTVLYLIVKTLVRINSRYMIAFGILERDLALYRIKTGDESFRISDKVKSETVNARPERLTLYFRENYKLNPRVAVSCETNAGSFSKNVFKVYRNKVAHLQTVTLLPAYIKEVGIGKVKSMFDVYHCVLLYGISETDFHDETLNAALKDNLEKCKEYCTACKDFIYALNIPFAYNAARYINLSNREKFIESYGK

SEQ ID NO: 72 - protein, 920 aa, unidentified
  source 1..920: note = uncultivated organism; note = MG103-25 effector
MATRKTIAKADGLKSTFYLEPEHLFMTSFGKGMHAKSEKDIQDRTVTNLNNTFTASVPVDSNWISINGKAGQCQVAEPKLQDQLHAKEIIESIYFGKEYSDNIHIQLAYNIMDIQKILAAYSGIVIYTVNRMNRDSDDKDFLGEFKTQNSYDMTRTISKAYKLRLLDKNRNGKGFFINGNVWRDNRNGELKWLNRQVNIYKQKKFPDQHWNYAIISDFLCSRGIHNMKILKKTYDCWNDFCTFCKSAKDSAYYFGNTFCDEKGNFDERKTFTLLRLLGTFRQDFIHGEQLGGSSLFRPAEKWPDDVTAMLNSLLDGRIENLNKNFCSQNRVNIRILEKIYPDISTKQLVREYYNFLIRKSFKNIGFSVRTLRETILAQEEEAHALTEKSYDSVRGKLYSLMDFVIYKFYLDREKDIEEMVSKLRFSISDEDKERVYQAEAERLWLYIQESIMKNIVPAMTSDSIKSLSKGKADAIENIEDYMEKPGDLSAFSKAIYAFSLFLDGKEINMLLSDLVSKFENIASFLDTLRFLNMDCSFTDNFVFFKDSAEIAGDLRFIQSIARMNKGKKAKKNTEVTVKKCQYKDSAAVLGETDESKINRLYNLKSSDSETPSGDNSLRNFIINNVINNNRFTYVVRFLKPADAREIMQCRPLIAYTLQKIPQSQIERYCRAVSIPFAKDECEKAVNELTKLLINVKLDNFVSVKQKASSGSKEAVQKEKYKAVISLYLTVLYLIVKTLVRINSRYMIAFGILERDLALYRIKTGDESFRISDKVKSETVNARPERLTLYFRENYKLNPRVAVSCETNAGSFSKNVFKVYRNKVAHLQTVTLLPAYIKEVGIGKVKSMFDVYHCVLLYGISETDFHDETLNAALKDNLEKCKEYCTACKDFIYALNIPFAYNAARYINLSNREKFIESYGK

SEQ ID NO: 73 - protein, 926 aa, unidentified
  source 1..926: note = MG103-26 effector; note = uncultivated organism
MSKNKKTAAKRMGVKSVLRHGEENLTITTFGKGNDAEIAVQTNIYGEGIITPYNTNRFTIEDIDETIEVVHGDLESLLNNPIENPGEDYLQLKKTLENEFFGKTFDNDNIHIQIIYNILDVMKILGLYVNDIIYSVNNLQEVANTQAPLADIVDNFDIVGKSLSEDKAKTNLERMNPYLGFFGDVFILLPKKDYGKSFSRAKDQEENDKMEIYKHNKSVLRALGAARQITGHVKKFTILFSKNDDLRKKMKKEYKIDWDVVRNSYKNRVDDINDNFLLHSRVNLHILYELLGATEMRDQIEVTEEYYKFSILKEGKNLGLNMKKIRENLIDQFYPEVKEKQHDSYRQKIYAICDYLIFRYINNSEELNDMVAVLRKTSDEPEKDKLYEQFALTVKNKISGLFEKFIKQFDQGFPVFCTNSIPDEYIQNVKLKVNQTPLIELLSFLCNFMDAKEINELLSAYIHKFENIQTFIDTLKKLGENVQFTDSYGMFNDYNGCRAGRIAQDLRVLASIGKMKPDLSDAKMALYKAAIEMLGGQNKENIDYLAKQWLDDNLFLPNAPKDTKHSFRNFIANNVIESRRFFYLVRYAKPKTVRTLMNNPKIIRYVLSRLPEEQVDSYYLNIDDMPLKNRPLSDKIKALTNRLAAFTFQSILEKRGTINEAAKPGCRNRVVERLKALTGLYLTVAFVAVKNLVKINARYYIAYFARERDEEMFTRKLVEAKENINSFSYPFINNEGKDDFNAIAAITKYFLDLEKKNDYKPDPGMPFDVEACRKHLNGLRRHFTKKWRDILQKNLEEGEKINATGYFFMEHRNAAAHLTVLAILPQYVEQFRANTPGEMISYFELYHFLLQKYLCDKSLQDTSPQLDIPDIYRQNIEKFSTPCLDWIKIAYAPLGYNLPRYKNLTIEALFDKDSEEGIKRENQKKK

SEQ ID NO: 74 - protein, 893 aa, unidentified
  source 1..893: note = uncultivated organism; note = MG103-27 effector
MSKNTKTQAKRMGVKSAFAYGEGRNLAITSFGRGNQANLEVNTDARGSDLHLPYKADSFMVKSIDEEIGLSRGKLESLLNNPAEACDTEDYLGLKGTLENEVFGKAYPNDTVRIQIIHNILDIQKILGLYVNDIIYCINNLQENEIPEDRIGLGLGDDKAKELLHKMQSFLSFFGDAFQLPGKKETSASQPDRTVEHNIDVLRILGTIRQQTIHFKDISFFFVRDLDENFHKKEGKWEIIQQNYRKLTERINKDFLEHSAMNLQILFEIFSAETPEAKIQLTEEYYRFSILKEGKNLGVNMTKIRERIIEAFCPGIKGKTYDSFRSKLYTIADFILFRKLSAPGVLDKWVQQLRRTSDEEAKEALYEAFSKTVWKKTQPLIEPFFKRYSRGFMKTEKAMLSPSMIEKVCLKADGEPFVQMMAFLCNFWEGKEVNEILSAYIHKFENIQAFLDTLHELGEEVTFTNFTLFNEPGMAERIAAQLRVLASIGKMKPDLSAAKRPLYKAAIETLGINDSKYITDEWLADNVLLGKEATEERKKDVNPFRNFIAGNVIGSRRFMYLVRYTKPKITRALMGNIQIVHYVLTRLPEKQIESYYGNISSFLDEKVPLQTKINRLASKLTRFSFQDYLKNTSNIIANSQREKNVEIERLKALTGLYLTVAYVAVKNLIKANARYYIAFWALDRDYHLLLKKDKETLDSLCIKYGENEKQKHNEALALTEYYLKKEGENDYKPAPGQLFDKEVCRRHLDSIRRHFSKKWREIFRKQIAEALAIDDTGYFPIYFRNDAAHLNVLSPKTVPFISQFRARQPGMQSYFELYHFLLQMKMFKSESVTFNLPEEYRKFLDSGVPCLDIIKIAYVSLGYNLPRYKNLTTEALFDEDSKTGKERANRRKG

SEQ ID NO: 75 - protein, 942 aa, unidentified
  source 1..942: note = MG103-28 effector; note = uncultivated organism
MGEYKIMDNVKIKGKSKAAGLKSTFFIDNDNYLMTSFDKGAKANIEKKISANERKISYERSPKKFDVNFITEKPEVAIKGHGIESEINLDNISKCENSLHAKDTIENIFFGKKFEDNIHIQLAYNIMDIQKVLAAYAAIVVNAINNLDPECGDNDILGMLPTQNRFKLASAIKKAYDNKLFKFDKNKNLINIDKIKQDLNWEISEDDLKKIPKTEKVINKIANNVEFIKKIKEKSYYFSDAFMNSEGKFDEEKAFEVICIMGNFRQYFFHGENKKFKDNIFYNLEERFDKEQIDLVSKILDSKIDKLNTNFLKTNKVNLVILNKIYIESDIQDEKIKQEKLKELCKQYFNFMILKEYKNMGFSIKKIRENILENKCTRFKEVRYDSVRNKLNNLLDFIIFKFYTENDPNVNKFVEELRSSMTEEEKNEMYKIESDMVWESIGKYVKGESKNSKGENTTGKNISVMSELYGGNINRLKKDKSLNSIMDNNNDDEIKKDLKIPEISSSFSKTIYCLSLFLDGKEINMLLTTLIHKFDNISSFIDVIKENEIDVNNKNKNDIFKNGFCLFNNSQKIANDLRIINSIARMNKGKKAVKCSGVNIKFFQYRDALYVLGGNEEDVKQLKKKMEKNKKKKNKKDNSLRNFLINNVINNNRFVYVARFIDLKDARDIMSSKILIKYVLESLEPKQITRYCKTINIKDDEHSIENLSEELLNVKYENFKDVKTEVKKGSEEEKKKEKYKALISLYLTVLYLIVKTLVRINTSYIIAHGVLERDITIFEDKIKSTNGKPSSTLVFEYFEKNGLLNKRIKNNLEHGKNYYDDNIFRIYRNNIAHMSVITHLPKFVKNYLNDDKYIPEPLKNEPIKSMFDLYHCILLDLILKNEKLKKNDKLKEFENLDSAQKEYRYVRVSKDFLYSLNKPFAYNPSRYINLSNREKFLENFGN

SEQ ID NO: 76 - protein, 1017 aa, unidentified
  source 1..1017: note = uncultivated organism; note = MG103-29 effector
MEEVKNVVFIRDYANYKKCDIVKYDSLSDNIKKMKGIFLSENEKIHEVLEKITNKNNIFFAKVKLVESILEGNKTEIKNPIMLKNIVEAINAKKSISIKVEEDIAINKKGLSVFYLSDGKENYGIVVIAEIAPIKGKAKAMGLKSSFIHNNSLILTTSASKNDNVIEKKIKDNKLVSEKSPKEFDVKEINDIKIELYKENKSALIDNQMHRKDEQTTVVRNDYLDKKDDLEIEFFGKKFNDNIHIQLIYNILDIKKIMGLYIDDIIYMFKNLDRTLDDDILGTLYYEGTLSSQLNDPTHKEKANKFINYFKNISKYLPYFPAAFTVNMVKVNGKMKKVYDEYDGYNIFRILSMIRQFVKHSSNNNSKNKVSGLFNLENNQNAKIDDLLAFIDKKYSAMIEKINTSFVKNSQKNIYLIKGALKQNSISDQELTNLVNAYYDFSFAKANKNLGFSFVRLRELLLEKLMLEINDEDKGKYYSIIDFVLFYILTTKYQNRIDKMVLKLRECRREEEKSQAYNSFADELLPLIKNYIDYKLINNIKHVKTDKNEAKYKNVFTSRNFNIQKNDNYFGKIIYFMTCFLNPKEINMLLTSLINKFENINSLISLLKSLGEKVEFTSDYQLFNNLDKIDVLNNLNIIMNLSRIQKNYNKIDKETKAVQTDKMFGDAFRIFGYIPSDKNDNYNEFRQLMDKVFESTAKSKRERGEKSNLIVNGKKVASLRNFIVSNIINSNNFRFVVTYNDPASCYKIMQNMTVVKFVLNNIPESQLKKYYQRMNLKYTNKKETIEYLANSIRNININRIIDSYETNVLGIKEITNLYLAVLFIITKQMVQINSYYSIGFSCLERDSSLYNIDLKNKKSKDSYLTLLNKFIEKGKLKSRAVKYMLLNEEEYHELFSDKEKGNSLIAKYRNSIQHLNALSKIGHYINDFNINKEMRSYFELYNYIVQRDLLSELEHEYGQAKPELLNKYINNLNNYHTYDKELMKIMNLPLGYNLARYKNLTIEDIFYDKYKQGIKVD

SEQ ID NO: 77 - protein, 1199 aa, unidentified
  source 1..1199: note = uncultivated organism; note = MG103-30 effector
MPHSNKKATNKVSSAKISGLKSTFVINDSKLLVTSFGNGNEAISEYEITKNDGKWETKIIENGNENRKIGISLNRIPDEDESKSEEESTDTVENNKFNKIYVKRIGIENINGQTDNPIAKHERKKMDQIGIRDKLEEYYFGETFDDNIHIQIAYCINDIEKMLAFYINDIAYSINNIIPEIRYDNNNETIIDSGFKGNDLLGSMSYQNPYFIYILSKNNTQPSHSLRAFKELINNAKVYGINVIMPQENTEQENTDKYKICLTEEQFYYLVNVVGYVRQCIAHNSNKNNLYCLYRFRRTTESEFGNDNNLNALFKSLTSDDYSDNNLKNNLIQYFKNYICSNFGEQREDEGIKKWLKEKYDMDDLTEEIFENILSQDDLINKSFYFAIDNSINTIKELFDKFYEDNVDYNTLNSNKKKEHMKSIKAVFKSSFLEPICKNGYIEVLNNIYNYKIENINMDFLNNSQINIAVILSIFDEKIKPENISKETLISEFYNFIIKKDYKYIGVSIKKLREYIIGKAYPNENSSNYNNISINRTKNKINLLTDFMIFHYYMHDDSLKNMIDECGLKLRMCIEEKNKDDVYAEYGEKIYKYYETIFTSIIEKNNYFNYDYKTFVEKFNLNDNNYNKYNKYKKQIANVRNEIINKLPDDGNYSYIKELNDKRYKATVIKTIDMKLYNYYKNMKSPNKNKKILDLMKSKDNYNYSYAAVKCLNNKGIETFLNEINSKVKNVETTIINSIKDIKSDNFELKTEELFFDLDYDGLNQDNNYRIFCHIIYAMTLFLDGKEINELLTKLSNKFDNIASFLDVINNKINDNSSDNNNIFKEEYQLFEKSKEIAEELRFINSFARMNYCLICNDENMKKALKILGCNQENTSITDNKKSNFIINNVIRTRSFMYITRYANMDTISNIIKNRSIVELVINNIPDKQIDKYYNSIKNIKNNQDKQTPTEETSNEDITLEKKKICLVDCLHEYSYDSIIDNNKGINRSVVKLYLTVLYLTTKNIVNINSRYFIGVDSLIKDYRLKGKLTEYEINNTSHPIDYYNITKEYIDSLSDDKINNTILKNTLNSLVGESANHNKEVEANKIYVLFRNCIEHLSAVRLIGSIDLNIDDISFSDIKFKRWFDLYQYIIQVNIFKNVTNDNNLLDNEKEISMEYLNKIKRYKKYSKDALKAMCLPFAYNLPRYKNLTTERLFDMNR

SEQ ID NO: 78 - protein, 1159 aa, unidentified
  source 1..1159: note = uncultivated organism; note = MG103-31 effector
MKEEKDFERSLEEPKFTAPLKNRLQGPLRDRLQGKKSNAKRIGVKSALAHGDNRLAITKCGQGNSAEIVFDEPNGGRDVVTPKPLEKAPFTIDYINEEIGFSGGYDSNELEAMLHNPTEKRDQDRLKLKPTIEMEIFGREFPGSTVHIQLAHNILDIQKILGLYINDVLFAINNLQASEFKLGCIKYDGAKDQVIKEPENLYKRINNIDDILGQTLDDDNEDDNTRKKQIKKLKGILFKLRPYLGFFGDAFKTYPKIENANDNKQQGKTENQGEELTDIDNHNLDVLRVLSALRHWAAHWGVNEFTQMDRNEEYPFFNINKTGDELLLGTKGKWNIVEDNYKTKINRINKDFLKNSKLNIQILCQLLNLHDEPEKKALIQDYYYFSILKQGKNLGVNITKLRENIIKQFHSELLDKVCDPHRQKINTITDYVIYRKLKASPEMVVQTVNRLQYSANEEQKNLVYKNLAISLWTQLKGILESFFKEVHCKSPRCVCDKYAKKIDKKLVDDIMLKSNDGIPFVKLMSFMCNFLEGKEINELLTAYIHKFENIQSFIDIIVQFGEPVEFVETYCAFNSLDNQFAKKVASQLRILASIGKMKPDLTDAKKPLYFAAVHTLGATDEQAEEFIEKYVLPNKSREDYETEEDYEKIKKSANPFRNFIANNVIESRQFQYLVRYSKPKIVRALMNNATIVRYALSRLPEKQIDSYYEQTPRVHKHAKRDEKLDVLTQTITGMTFETVSDNRNNIAEKSQKLQEIKELSGEIKSAENQIKQSKASVKQLKEDINNKKNQIKQSKSQVNKLEEKIEPLKAQVNLYLTVAYVAIKQLVKINARYYIAYQIFERDLQLMQTKVDDESTKRVSKHFIPYMHKDDKGNDKEDFVKHFGLIRYFLEQDENLFNEIGQWKSPDKATWYAHLNEMKKMRHFSPKCKEYLEREVNESLEISDTGFLPVAVRNITEHLILLMLIQDYVGSFHKNASSGPMTSYFELYHFLMQSLLHQVEGLKLDKYSKDNQNGVVDTRLVHIAYVPLGYNLARYKNLTIEALFDEDGVKGQNFVANKCYKQTFNRIATAKRRGKPDNVIEALKKDFIDQYGSEEEAQKAVRLGELWGYQDEARECGLKGKPFPEMVIRMMKFRKFTEAEISEVKRLYEVAKRKAPQNQ

SEQ ID NO: 79 - protein, 1023 aa, unidentified
  source 1..1023: note = uncultivated organism; note = MG103-32 effector
MKKVSEENKECDRKDAAKAFGKMKTKAKASGLKSVFLYDNGKDDNGKGNRLAVISFRDDERGTSAGGRVRYPHAEKVTTSDGKSVFGEEPLMFESVAPRSDAIELTDAGGRTFEVANPSRFSSKSDYIGLKSKLEEEFFGKAYGDDTFRVQIAYNIVDIRKILVPYVSNVIHSVLNISGKVSGFLGNGDSDYIGTMDFRADFQQFMKKADCFFGNGCRCIGKCADCVSYRKTADSKAKDPGKVCEKFYKLYCTLKSESAASGSFRKLGYFFPGTFEYYKPAKGKQVGIKANGVNDANGIPEGTLRRNYDCLRFLSMMRQFALHKNVLVNSGLDYEGRDWESCETLLFKAGAILKESGLRSFVDDKMNMVMTYLNAMPENLSNNIFMLAEYYGDKKSRQEILKEYCTLVIRKENRNLGVNLRKIRELIIDRHLVGLRSKEWDSCRQKAYAVLDFIIYDFIKDNEEMRIRVINDLRGSISEGCKEAVYEKAADLIFSDSSVDFPKALRKLEDQREAGFNGGKVSKSKKDDENCQEESKKKELLPEECRISPDDVSGFAKVVYLLGLFLENKDINILFDSLISKFDSIADLVDCVRFSDGLGALEFGGKYFGCDMDFRRLSSDMRLAKSLGKLCPNKDDYEAELFKDALLMLGIPQDEINGENWQEYDGRIFEAKKGNKNIRNFIINNVLKSRWFYYLSKTFRPADCHKLIKNESVVRFAVESLPDSQIDRYNGLTGGNDGIGINEKKDSIVNTLIEFSFKSMMDNIESLTDKEKKDQSIGSKAERNKKIVTLYLTVLYLIVKGISAVNVRFQIAFSCLERDKFFLEPKESDRKDYLSLVRKFLDEDGRIAEEYRIERKGIRDGNSSEEEKKALYSRADSKLKSMHYSLKYFDIIKGNLAEAENLDRIVKDSSKEPIGKNLDRAYVKFRNQVMHFDVLSKLPDIASEEAFQRMKFKSYYSVYRHILIKSLWKSVLCNSDFCKERFADFSQSDSIQRNLMWVLNIPFAYNLARYKNLSIEDIFNDKE

SEQ ID NO: 80 - protein, 1059 aa, unidentified
  source 1..1059: note = MG103-33 effector; note = uncultivated organism
MKSCVGKYKRMGKIKEKERQEAEIRKKGEEKRAEDMLRAKIEKEKKAEADKFYHKTDAKAKGLKSVFLYDDGNGPKLNVVSFLDDRDEDDDVKYPHIEMFSDTEGTDKKLELRKFEQISYGGEEKEKQKTISIRVKNKDGQLESIFYPTALLDKGDYLGIKKILERKYFGGEFENDNLRMQIAYNFLDIKKILSLYISYIVGSISNIVSGYYESNGDKKNRIDDDFSRRDVIGNIASFSDYTDFMSYNLKLKNEPEKNKDKIEKNDNLEGFLIRSASDGCMRKLNFFFPKVFKTVKSNNSGKTDSDKKELNKAVEHNYDVLRAVSFIRQCVEHEEVSFSSVFKKRNEYEYTDLFLNGEDALESTKLKGVFESMFDKRIEKLKSVPKNLGNTYYILKRYYGSKTEKEKRILFERLCRNKIFKDYKNVGVNIGKLREFMVARHFDSLTEKEWDSCRSLKINKIIDFVLYEYLVAKGQNILEDMIDELRSSKQESLEYDVKDKIYWEYAERIYKGIGNVDALVGIIKKEKEKKEDEGGPFSGKDASDDSLLSSLPESCARGFEGVSAFAKIICFMGEFLDNKDINILIDTLISKFDSMADLIDCVGYAKKQSAISFAIKKDPSGINFRKISMDLRFVKNLEKMPLLESKNYEAELYKDALMMLGVDESSIRDKARWEVIENRIYKKEKNKKTLKNFLINNVIKSRWFGYVGRNYNPAKCRLLMSNRSLLNFVFKDIPETQIDRYCGTIGCSCIGSLQSKIDSLIDILLNYSFLEKMNEIENLTDHEKKEQEEGSRFEKLKKTATLYLTVLYLIVRGLLRVNARFMIAFSCLDRDKELLKEELGYDKKSLPKKKHELLTEWYLNRDEQIYNDFLAEKRKATDPSISDEDRDRIYSEAGKIYKRMHFKSRVYKYIRENYQEAKELGLTDRSDPSGAELTFVKFRNYVAHFRVLAEVANLLEDECFRKRFSEIEFASFYSLYRHFLLVSLIKGCLFGNQEFFDRKFADYETNLRTQEKMMWELNIPFAYNLARYKNLSIENLFNDRDIRQREKAQKNKSNETNTL

SEQ ID NO: 81 - protein, 977 aa, unidentified
  source 1..977: note = uncultivated organism; note = MG103-34 effector
MSNRKQIKTDAKSNGLKSVFLYNEGHGGKLGVVSFEDDEMKNGFGDDLKFPHIEKITSKDGGEEFWFVRKMFKDVDSDVANVNIKSLSGEGFSVSNPAHSLKKSDYIGLKEVLEDKYFGKSFEDDTIRMQVIYNFLDIKKILALYISRIIFSVGCIIDSKKPEYNFSGSDIIGNMHSSTTFQKFENDVLNYKSYKKRGQSAKIKFYDLLVEEMSDNEKAGGSVFRKLSYFFPGVFEMRMFLKKDKKGNILQQADTDDDIKAKTRNNYDCFRVLSLVRQLVVHQDAKFDRDSNSNKLGYEYEDLFFNARRLFNEIKLTEIIDDRFSRGIDEMNDELSRNLKNNLYILSKYDPSRKTNELAEKYYRFIVYKKNRNIGIDARAVRENILKNHIKGLTDQKWDNCRKKIYMVVDFCIYEYFQEHKAVVDEIRGRLRFAKEEGDKDAIYLGVARKIYESGDIDFRAIESILETEAAETDDHKPFSGEVEYSIDGFKLKSKDNYGISNFSNFSKLMYFLALLLENKDKNILLDRLINKFDAIADLIDCIKYNDKLKNIEFAQKYSDFPISFRNISGELRLVKNISRMKCRVDDCGPDFFRDALLILGMSKERVGKEWVEIKKRIYDAVDGHKNLRNFIMKNVVKSKWFAYLSKSYVPYKCSMLMKNRSMLEFVIDKRPDEQIERYFDLVFGLNKKPNSMESSVPSLTITEKKEKLVKILLDFSFESIMVEVEKSADKDRREIHKRLVTLYLTVLYLMVKGIVDVNSRFVIAFSCYDRDSKLILKEDDSIGNLGLTEYFISHDDDILSKYYIERNKIRDDVSLSKEDKKLRYSEIDGKFGKTHYNTRFLKIIKDNMKETKDIPVVKGQNGKESGVCNVFRNIIAHYNVLANMDRYIGDQRFCSMSFKSFYSVFRYLLQRTVYDRCGGEVKLYIDSKYGDFASNEETQKKLMWILNIPFAYNLPRYKNLSIENLFNERDGNNDNG

SEQ ID NO: 82 - protein, 1059 aa, unidentified
  source 1..1059: note = uncultivated organism; note = MG103-35 effector
MKSCVGKYKRMGKIKEKERQEAEIRKKGEEKRAEDMLRAKIEKEKKAEADKFYHKTDAKAKGLKSVFLYDDGNGPKLNVVSFLDDRDEDDDVKYPHIEMFSDTEGTDKKLELRKFEQISYGGEEKEKQKTISIRVKNKEGQLESIFYPTALLDKGDYLGIKKILERKYFGGEFENDNLRMQIAYNFLDIKKILSLYISYIVGSISNIVSGYYESNGDKKNRIDDDFSRRDVIGNIASFSDYTDFMSYNLKLKNEPEKNKDKIEKNDNLEGFLIRSASDGCMRKLNFFFPKVFKTVKSNNSGKTDSDKKELNKAVEHNYDVLRAVSFIRQCVEHEEVSFSSVFKKRNEYEYTDLFLNGEDALESTKLKGVFESMFDKRIEKLKSVPKNLGNTYYILKRYYGSKTEKEKRILFERLCRNKIFKDYKNVGVNIGKLREFMVARHFDSLTEKEWDSCRSLKINKIIDFVLYEYLVAKGQNILEDMIDELRSSKQESLEYDVKDKIYWEYAERIYKGIGNVDALVGIIKKEKEKKEDEGGPFSGKDASDDSLLSSLPESCARGFEGVSAFAKIICFMGEFLDNKDINILIDTLISKFDSMADLIDCVGYAKKQSAISFAIKKDPSGINFRKISMDLRFVKNLEKMPLLESKNYEAELYKDALMMLGVDESSIRDKARWEVIENRIYKKEKNKKTLKNFLINNVIKSRWFGYVGRNYNPAKCRLLMSNRSLLNFVFKDIPETQIDRYCGTIGCSCIGSLQSKIDSLIDILLNYSFLEKMNEIENLTDHEKKEQEEGSRFEKLKKTATLYLTVLYLIVRGLLRVNARFMIAFSCLDRDKELLKEELGYDKKSLPKKKHELLTEWYLNRDEQIYNDFLAEKRKATDPSISDEDRDRIYSEAGKIYKRMHFKSRVYKYIRENYQEAKELGLTDRSDPSGAELTFVKFRNYVAHFRVLAEVANLLEDECFRKRFSEIEFASFYSLYRHFLLVSLIKGCLFGNQEFFDRKFADYETNLRTQEKMMWELNIPFAYNLARYKNLSIENLFNDRDIRQREKAQKNKSNETNTL

SEQ ID NO: 83 - protein, 1202 aa, unidentified
  source 1..1202: note = uncultivated organism; note = MG103-36 effector
MPHSNKQDTKKVSSAKISGLKSTFVINENKLLVTSFGNGNEAIPEYKLTKDGENWEAKIISNGNENRKIEISLTRIPDEDESKSEEDSNDTAEHNEFDKIYVKRIGKENIKGQTDNPIAKQERKMDQIGLRDKLEEYYFGETFDDNIHIQIAYCIRDIDKMLSQHINDIKSSINNLVPEIQFNNITIPEGNEDNDLIAYFKYNKKFANFLNDANMNNSKSIAKPFDELCNSGYLGYYGLTVVANAEEGLTKEDLYYLFCVLSFVRQSLAHPEKNNNLFNLCWSIDQRHGHTYDDILGVLNKLYDNKINEIDVGFIKKQKRNIKIISDVLKQSESDINSLIQIYYRFYLNNEYKNLGFSIKKLREEVLNIFMEKNEIDSLAYDKYKRNINNLVDFIIYYDFITNNKIDEIVEQLRASAVNEDEKNKIYKDYAEKNTESIYQEKIKLILKLVDINSNALKIYTANENFDYEGFVDIRESNGFNVSKIREILKKEKKLNELYSEDNIAVYRIIDLIVDYEIYSSCKDDIDTIKTNAKNSLNTFKNKCYNELGNKYLKKFKESKLYKKLNEGNGYFLNNDKNFYYVNDKYGQNDGIINNDVITNIKSLFEKFDKIEENDRITNQYINFFKLKNVLKEKEIIQKIYDKYNEDEHIKKIDKLISFKIYYDQYNLKMKSNNKDVELIKFYKNKINKKNFNKCLADECYSKIENDIENIKNKIINSTVSALFVDEDTIRNAVNDIKISNCNDNNDNSDIFCKMIYAITLFMDGKEINELLTGLINIFDNISSFMDVLEKNRIEVSFTDKFSIFNDSKKIVDNLKIINSIARVNKNANVKEYSKQKKALYLLGCKNDDIISIIGRKTDNRIDRNIRANRNYINNNILKSPKYTYLARYCNTSMISNIVKNKSIVRLVLNNMPQEQILSYNDILNEIVQNNNQTSVNLSAETDDISAENVINLLTELLTDFNYNSIFLDEKKELYKSFLNLYMTVLYIAAKNISHINSRYFIAFDMFLRDNNTSEDEQYYEIIKESTNALTHKDKENNKKTFCKCFLGQDSTDISATEANIDNSKLDNIINNLNYIIKAFRNSIEHMTVVRSIGDPDLDENFEIISDVFTDETLSDLEFKDVEFKRWFDVYHYILQIIIFKKIIGNSNHTKDQNGFAEKYLRSIIKYKKYSKDALKAMCLPFAYNLPRYKNLTTESLFDMNR

SEQ ID NO: 84 - protein, 909 aa, unidentified
  source 1..909: note = uncultivated organism; note = MG103-37 effector
MGDKAAIEKKVEGQSVLDMRNPHAFELKLKERQLQIIGKNKINTVRENPLITAPSKRQMIREERMKRAIEENRPYHIIKAAGEDDLRAKSQLEKQYFGREFNDNIKIQIIYNILDINKIIAPYINDIIYSMNNLSRNERQEDDNKIDIIGNLISTGDYVSFRTPSPYPDKEKNNKRNRDNYDNFLKAAKPYMRYYGKVFIKEVKKTRIKEEKRDKLEIIHRSDGEVFTIFQILSFVRQNLMHSGKNASSIFNIEDYSETFRTFLKEIYRQKINEINKGFIDNNSHTNFSILYDLYGLEENTKNTKEIVQEFYDFVIKADSKNIGFSLKKLREHMLDLPEAVELKNKQFDTVRSKLYTMLDFMIYHHYVSNQTIINEIVEHLRTTLTDEAKEYIYLSQAKILWNALGSIVFEKLVPMMNGDSLKEMKKENKSYKLPESLISTVLAKPDANVFSCLIYFLTLFLDGKEINEMVSNLITKFENIDSLLEVDKKVYRDTNEELDLESLIRGFKGKVTKKIDDQKLNGELLGVFKLFNDAKKISEELKIIKNITRMDKEIYPSEGVFLDAAKVLGIKKQNLDYDSDTLANDLDSDFNKNIVNVINGTKEDRNLRNFIINNVIKSRRFQYIARHMNMNHVKKLAQSEALNRFVLTKMNDTKIIDRYYESVSGQVVTLNQKAKIDYLVKKLHGLTFEDFIDVKQKVSENTLQSKEKAQKQALVGLYLTVLYLIYKNLVNINARYTTAFYCLERDSALKGLEIKWDDLESCKGLAKHFIEKNYLPVRKAGLLKANLTHLDNENGFKFYRNQVTHLNAIRVAHMYINDIKFVDSYFALYHYVMQRHLLTSLQKYPDNQGFVIESLVKAKTNGVYSKDFLHVIHAPFGYNSARYKNLSIQDLFDKNEVKPEVNLPSTND

SEQ ID NO: 85 - protein, 281 aa, synthetic construct
  source 1..281: note = GFP-PEST reporter
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLSHGFPPEVEEQDDGTLPMSCAQESGMDRHPAACASARINV

SEQ ID NO: 86 - RNA, 123 nt, synthetic construct
  source 1..123: mol_type = other RNA; note = Non-Targetting Chemically Synthesized RNA Guide for 103-2
  modified_base 1: mod_base = OTHER; note = 2'-O-Methyladenosine
  modified_base 2: mod_base = OTHER; note = 2'-O-Methyladenosine
  modified_base 3: mod_base = cm
  modified_base 121: mod_base = um
  modified_base 122: mod_base = um
  modified_base 123: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 120^121: mod_base = OTHER; note = phosphorothioate bond
  modified_base 121^122: mod_base = OTHER; note = phosphorothioate bond
  modified_base 122^123: mod_base = OTHER; note = phosphorothioate bond
aaccncncntnancncnanancntngngntncngngngngntntntngnananancnananangnangnanancnantngncnancnantngnanangncntngntnancnantngntntttt

SEQ ID NO: 87 - RNA, 123 nt, synthetic construct
  source 1..123: mol_type = other RNA; note = Non-Targetting Chemically Synthesized RNA Guide for 103-3
  modified_base 1: mod_base = cm
  modified_base 2: mod_base = OTHER; note = 2'-O-Methyladenosine
  modified_base 3: mod_base = cm
  modified_base 121: mod_base = um
  modified_base 122: mod_base = um
  modified_base 123: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 120^121: mod_base = OTHER; note = phosphorothioate bond
  modified_base 122^123: mod_base = OTHER; note = phosphorothioate bond
  modified_base 121^122: mod_base = OTHER; note = phosphorothioate bond
cactngngntngncnananantntntngncnancntnangntncntnanananancnananangnangnanancnantngncnancnantngnanangncntngntnancnantngntntttt

SEQ ID NO: 88 - RNA, 123 nt, synthetic construct
  source 1..123: mol_type = other RNA; note = Non-Targetting Chemically Synthesized RNA Guide for 103-6
  modified_base 1: mod_base = cm
  modified_base 3: mod_base = cm
  modified_base 121: mod_base = um
  modified_base 122: mod_base = um
  modified_base 123: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 120^121: mod_base = OTHER; note = phosphorothioate bond
  modified_base 121^122: mod_base = OTHER; note = phosphorothioate bond
  modified_base 122^123: mod_base = OTHER; note = phosphorothioate bond
  modified_base 2: mod_base = OTHER; note = 2'-O-Methyladenosine
caccncngntngncnananananantngncnangngngngntncntnanananancnananangnangnanancnantngncnancnantngnanangncntngntnancnantngntntttt

SEQ ID NO: 89 - RNA, 303 nt, synthetic construct
  source 1..303: mol_type = other RNA; note = Non-Targetting Chemically Synthesized RNA Guide for 103-7
  modified_base 1: mod_base = OTHER; note = 2'-O-Methyladenosine
  modified_base 2: mod_base = OTHER; note = 2'-O-Methyladenosine
  modified_base 3: mod_base = cm
  modified_base 301: mod_base = um
  modified_base 302: mod_base = um
  modified_base 303: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 300^301: mod_base = OTHER; note = phosphorothioate bond
  modified_base 301^302: mod_base = OTHER; note = phosphorothioate bond
  modified_base 302^303: mod_base = OTHER; note = phosphorothioate bond
aaccncncntnancncnanancntngngntncngngngngntntntngnananancnananangnangnanancnantngncnancnantngnanangncntngntnancnantngnanancncncncntnancncnanancntngngntncngngngngntntntngnananancngnanangngncnancncngntntngnancnanancncnancncnancntntntnanangnanancncncncntnancncnanancntngngntncngngngngntntntngnananancntntttt

SEQ ID NO: 90 - RNA, 339 nt, synthetic construct
  source 1..339: mol_type = other RNA; note = Non-processed spacer array with 103-2 repeats targetting eGFP at positions 1 and 2
  modified_base 1: mod_base = cm
  modified_base 2: mod_base = um
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ctacntnancnancntngngntngncnananantntntngncnancntnangntncntnanananancngntnanantntntncncncnangntnangntnancnananantnananantntntngnancntnancntnancnancntngngntngncnananantntntngncnancntnangntncntnanananancnangntngntntngntntnancncnananangntntngngncncnantngngngnancnancntnancntnancnancntngngntngncnananantntntngncnancntnangntncntnanananancntntttt

SEQ ID NO: 91 - RNA, 339 nt, synthetic construct
  source 1..339: mol_type = other RNA; note = Non-processed spacer array with 103-2 repeats targetting eGFP at positions 3 and 4
  modified_base 1: mod_base = cm
  modified_base 2: mod_base = um
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ctacntnancnancntngngntngncnananantntntngncnancntnangntncntnanananancncngngnantnancncngnangnangnananancnantntngntnancncncncnantnancntnancntnancnancntngngntngncnananantntntngncnancntnangntncntnanananancngncntntntntnananangnananantncnantngntntngntntntncnantnantngncntnancntnancnancntngngntngncnananantntntngncnancntnangntncntnanananancntntttt

SEQ ID NO: 92 - RNA, 339 nt, synthetic construct
  source 1..339: mol_type = other RNA; note = Non-processed spacer array with 103-2 repeats targetting eGFP at positions 5 and 6
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
gaacntnancnancncncngntngncnananananantngncnangngngngntncntnanananancngncncnangntnananancnangntntncntntncncncncntntntngngnananancngnanancntnancnancncncngntngncnananananantngncnangngngngntncntnanananancntncncncncnantncncnanantntncncnancntnanananantncngngnanancntngnanancntnancnancncncngntngncnananananantngncnangngngngntncntnanananancntntttt

SEQ ID NO: 93 - RNA, 339 nt, synthetic construct
  source 1..339: mol_type = other RNA; note = Non-processed spacer array with 103-2 repeats targetting eGFP at positions 7 and 8
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
gaacntnancnancncncngntngncnananananantngncnangngngngntncntnanananancncntncncntngnangnancntngnanananantntntnantngntncncnantntnanangnanancntnancnancncncngntngncnananananantngncnangngngngntncntnanananancngntnanangntntntncncncnantnantngntnangncngntncntncncntntncncngnanancntnancnancncncngntngncnananananantngncnangngngngntncntnanananancntntttt

SEQ ID NO: 94 - RNA, 339 nt, synthetic construct
  source 1..339: mol_type = other RNA; note = Non-processed spacer array with 103-3 repeats targetting eGFP at positions 1 and 2
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
gaacntnancnancncncngntngncnananananantngncnangngngngntncntnanananancngntnanantntntncncncnangntnangntnancnananantnananantntntngnangnanancntnancnancncncngntngncnananananantngncnangngngngntncntnanananancnangntngntntngntntnancncnananangntntngngncncnantngngngnancnangnanancntnancnancncncngntngncnananananantngncnangngngngntncntnanananancntntttt

SEQ ID NO: 95 - RNA, 339 nt, synthetic construct
  source 1..339: mol_type = other RNA; note = Non-processed spacer array with 103-3 repeats targetting eGFP at positions 3 and 4
  modified_base 1: mod_base = gm
  modified_base 3: mod_base = OTHER; note = 2'-O-Methyladenosine
  modified_base 338: mod_base = um
  modified_base 337: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 2: mod_base = OTHER; note = 2'-O-Methyladenosine
gaacntnancnancncncngntngncnananananantngncnangngngngntncntnanananancncngngnantnancncngnangnangnananancnantntngntnancncncncnantnangnanancntnancnancncncngntngncnananananantngncnangngngngntncntnanananancngncntntntntnananangnananantncnantngntntngntntntncnantnantngngnanancntnancnancncncngntngncnananananantngncnangngngngntncntnanananancntntttt

SEQ ID NO: 96 - RNA, 339 nt, synthetic construct
  source 1..339: mol_type = other RNA; note = Non-processed spacer array with 103-3 repeats targetting eGFP at positions 5 and 6
  modified_base 1: mod_base = gm
  modified_base 2: mod_base = gm
  modified_base 3: mod_base = um
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ggtcnanantnancncncntntncntnanananantntnangnanangngngntncntnanananancngncncnangntnananancnangntntncntntncncncncntntntngngnananancngngntncnanantnancncncntntncntnanananantntnangnanangngngntncntnanananancntncncncncnantncncnanantntncncnancntnanananantncngngnanancntngngntncnanantnancncncntntncntnanananantntnangnanangngngntncntnanananancntntttt

SEQ ID NO: 97 - RNA, 339 nt, synthetic construct
  source 1..339: mol_type = other RNA; note = Non-processed spacer array with 103-3 repeats targetting eGFP at positions 7 and 8
  modified_base 1: mod_base = gm
  modified_base 2: mod_base = gm
  modified_base 3: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 337: mod_base = um
  modified_base 338: mod_base = um
  modified_base 339: mod_base = um
ggtcnanantnancncncntntncntnanananantntnangnanangngngntncntnanananancncntncncntngnangnancntngnanananantntntnantngntncncnantntnanangngntncnanantnancncncntntncntnanananantntnangnanangngngntncntnanananancngntnanangntntntncncncnantnantngntnangncngntncntncncntntncncngngntncnanantnancncncntntncntnanananantntnangnanangngngntncntnanananancntntttt

SEQ ID NO: 98 - RNA, 339 nt, synthetic construct
  source 1..339: mol_type = other RNA; note = Non-processed spacer array with 103-6 repeats targetting eGFP at positions 1 and 2
  modified_base 1: mod_base = gm
  modified_base 2: mod_base = gm
  modified_base 3: mod_base = um
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ggtcnanantnancncncntntncntnanananantntnangnanangngngntncntnanananancngntnanantntntncncncnangntnangntnancnananantnananantntntngnangngntncnanantnancncncntntncntnanananantntnangnanangngngntncntnanananancnangntngntntngntntnancncnananangntntngngncncnantngngngnancnangngntncnanantnancncncntntncntnanananantntnangnanangngngntncntnanananancntntttt

SEQ ID NO: 99 - RNA, 339 nt, synthetic construct
  source 1..339: mol_type = other RNA; note = Non-processed spacer array with 103-6 repeats targetting eGFP at positions 3 and 4
  modified_base 1: mod_base = gm
  modified_base 2: mod_base = gm
  modified_base 3: mod_base = um
  modified_base 337: mod_base = um
  modified_base 338: mod_base = um
  modified_base 339: mod_base = um
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 1^2: mod_base = OTHER; note = phosphorothioate bond
ggtcnanantnancncncntntncntnanananantntnangnanangngngntncntnanananancncngngnantnancncngnangnangnananancnantntngntnancncncncnantnangngntncnanantnancncncntntncntnanananantntnangnanangngngntncntnanananancngncntntntntnananangnananantncnantngntntngntntntncnantnantngngngntncnanantnancncncntntncntnanananantntnangnanangngngntncntnanananancntntttt

SEQ ID NO: 100 - RNA, 339 nt, synthetic construct
  source 1..339: mol_type = other RNA; note = Non-processed spacer array with 103-6 repeats targetting eGFP at positions 5 and 6
  modified_base 1: mod_base = cm
  modified_base 2: mod_base = um
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ctatntnancnancntnancnancncncncntntntntnancnangngngngntncntnanananancngncncnangntnananancnangntntncntntncncncncntntntngngnananancncntnantntnancnancntnancnancncncncntntntntnancnangngngngntncntnanananancntncncncncnantncncnanantntncncnancntnanananantncngngnanancntncntnantntnancnancntnancnancncncncntntntntnancnangngngngntncntnanananancntntttt

SEQ ID NO: 101 - RNA, 339 nt, synthetic construct
  source 1..339: mol_type = other RNA; note = Non-processed spacer array with 103-6 repeats targetting eGFP at positions 7 and 8
  modified_base 1: mod_base = cm
  modified_base 2: mod_base = um
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ctatntnancnancntnancnancncncncntntntntnancnangngngngntncntnanananancncntncncntngnangnancntngnanananantntntnantngntncncnantntnanancntnantntnancnancntnancnancncncncntntntntnancnangngngngntncntnanananancngntnanangntntntncncncnantnantngntnangncngntncntncncntntncncncntnantntnancnancntnancnancncncncntntntntnancnangngngngntncntnanananancntntttt

SEQ ID NO: 102 - RNA, 339 nt, synthetic construct
  source 1..339: mol_type = other RNA; note = Non-processed spacer array with 103-7 repeats targetting eGFP at positions 1 and 2
  modified_base 1: mod_base = cm
  modified_base 2: mod_base = um
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ctatntnancnancntnancnancncncncntntntntnancnangngngngntncntnanananancngntnanantntntncncncnangntnangntnancnananantnananantntntngnancntnantntnancnancntnancnancncncncntntntntnancnangngngngntncntnanananancnangntngntntngntntnancncnananangntntngngncncnantngngngnancnancntnantntnancnancntnancnancncncncntntntntnancnangngngngntncntnanananancntntttt

SEQ ID NO: 103 - RNA, 339 nt, synthetic construct
  source 1..339: mol_type = other RNA; note = Non-processed spacer array with 103-7 repeats targetting eGFP at positions 3 and 4
  modified_base 1: mod_base = cm
  modified_base 2: mod_base = um
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
ctatntnancnancntnancnancncncncntntntntnancnangngngngntncntnanananancncngngnantnancncngnangnangnananancnantntngntnancncncncnantnancntnantntnancnancntnancnancncncncntntntntnancnangngngngntncntnanananancngncntntntntnananangnananantncnantngntntngntntntncnantnantngncntnantntnancnancntnancnancncncncntntntntnancnangngngngntncntnanananancntntttt

SEQ ID NO: 104 - RNA, 339 nt, synthetic construct
  source 1..339: mol_type = other RNA; note = Non-processed spacer array with 103-7 repeats targetting eGFP at positions 5 and 6
  modified_base 1: mod_base = gm
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 338^339: mod_base = OTHER; note = phosphorothioate bond
  modified_base 337^338: mod_base = OTHER; note = phosphorothioate bond
  modified_base 2: mod_base = OTHER; note = 2'-O-Methyladenosine
gaacntnancnancncncntntnancngnanancntncngntnangngngngntncntngnananancngncncnangntnananancnangntntncntntncncncncntntntngngnananancngnanancntnancnancncncntntnancngnanancntncngntnangngngngntncntngnananancntncncncncnantncncnanantntncncnancntnanananantncngngnanancntngnanancntnancnancncncntntnancngnanancntncngntnangngngngntncntngnananancntntttt

SEQ ID NO: 105 - RNA, 339 nt, synthetic construct
  source 1..339: mating_type = Non-processed spacer array with 103-7 repeats targetting eGFP at positions 7 and 8; mol_type = other RNA
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 337: mod_base = um
  modified_base 338: mod_base = um
  modified_base 339: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 336^337: mod_base = OTHER; note = phosphorothioate bond
  modified_base 337^338: mod_base = OTHER; note = phosphorothioate bond
  modified_base 338^339: mod_base = OTHER; note = phosphorothioate bond
gaacntnancnancncncntntnancngnanancntncngntnangngngngntncntngnananancncntncncntngnangnancntngnanananantntntnantngntncncnantntnanangnanancntnancnancncncntntnancngnanancntncngntnangngngngntncntngnananancngntnanangntntntncncncnantnantngntnangncngntncntncncntntncncngnanancntnancnancncncntntnancngnanancntncngntnangngngngntncntngnananancntntttt

SEQ ID NO: 106 - DNA, 328 bp, synthetic construct
  source 1..328: mol_type = other DNA; note = DNA encoding for non-processed guide and repeat array for 103-2 guides 1,2,3,and 4 targetting eGFP cloned into and expressed by plasmid.(includes restriction digest overhangs)
ccaccttgttggggtcaatacccttctaaaattagaagggtctaaaacgccagtaaacagttcttcccctttggaaacggtcaatacccttctaaaattagaagggtctaaaactccccatccaattccactaaaatcggaactggtcaatacccttctaaaattagaagggtctaaaacctcctgagactgaaaatttatgtccattaaggtcaatacccttctaaaattagaagggtctaaaacgtaatttcccagtagtacaaataaatttgaggtcaatacccttctaaaattagaagggtctaaaacttttttttgcggccgc

SEQ ID NO: 107 - DNA, 328 bp, synthetic construct
  source 1..328: mol_type = other DNA; note = DNA encoding for non-processed guide and repeat array for 103-2 guides 5,6,7,and 8 targetting eGFP cloned into and expressed by plasmid. (includes restriction digest overhangs)
ccaccttgttggggtcaatacccttctaaaattagaagggtctaaaacgtaatttcccagtagtacaaataaatttgaggtcaatacccttctaaaattagaagggtctaaaacagtgttgttaccaaagttggccatgggacaggtcaatacccttctaaaattagaagggtctaaaaccggataccgagagaaacattgtaccccataggtcaatacccttctaaaattagaagggtctaaaacatagtccgttcttgcacatacccctcaggcggtcaatacccttctaaaattagaagggtctaaaacttttttttgcggccgc

SEQ ID NO: 108 - DNA, 328 bp, synthetic construct
  source 1..328: mol_type = other DNA; note = DNA encoding for non-processed guide and repeat array for 103-3 guides 1,2,3,and 4 targetting eGFP cloned into and expressed by plasmid. (includes restriction digest overhangs)
ccaccttgttggctattacactacaccccttttacaggggtctaaaacgccagtaaacagttcttcccctttggaaacctattacactacaccccttttacaggggtctaaaactccccatccaattccactaaaatcggaactctattacactacaccccttttacaggggtctaaaacctcctgagactgaaaatttatgtccattaactattacactacaccccttttacaggggtctaaaacgtaatttcccagtagtacaaataaatttgactattacactacaccccttttacaggggtctaaaacttttttttgcggccgc

SEQ ID NO: 109 - DNA, 328 bp, synthetic construct
  source 1..328: mol_type = other DNA; note = DNA encoding for non-processed guide and repeat array for 103-3 guides 5,6,7,and 8 targetting eGFP cloned into and expressed by plasmid.(includes restriction digest overhangs)
ccaccttgttggctattacactacaccccttttacaggggtctaaaacgtaatttcccagtagtacaaataaatttgactattacactacaccccttttacaggggtctaaaacagtgttgttaccaaagttggccatgggacactattacactacaccccttttacaggggtctaaaaccggataccgagagaaacattgtaccccatactattacactacaccccttttacaggggtctaaaacatagtccgttcttgcacatacccctcaggcctattacactacaccccttttacaggggtctaaaacttttttttgcggccgc

SEQ ID NO: 110 - DNA, 328 bp, synthetic construct
  source 1..328: mol_type = other DNA; note = DNA encoding for non-processed guide and repeat array for 103-6 guides 1,2,3,and 4 targetting eGFP cloned into and expressed by plasmid. (includes restriction digest overhangs)
ccaccttgttgggaactacacccttacgaactcgtaggggtctgaaacgccagtaaacagttcttcccctttggaaacgaactacacccttacgaactcgtaggggtctgaaactccccatccaattccactaaaatcggaactgaactacacccttacgaactcgtaggggtctgaaacctcctgagactgaaaatttatgtccattaagaactacacccttacgaactcgtaggggtctgaaacgtaatttcccagtagtacaaataaatttgagaactacacccttacgaactcgtaggggtctgaaacttttttttgcggccgc

SEQ ID NO: 111 - RNA, 328 nt, synthetic construct
  source 1..328: mol_type = other RNA; note = DNA encoding for non-processed guide and repeat array for 103-6 guides 5,6,7,and 8 targetting eGFP cloned into and expressed by plasmid.(includes restriction digest overhangs)
ccaccttgttgggaactacacccttacgaactcgtaggggtctgaaacgtaatttcccagtagtacaaataaatttgagaactacacccttacgaactcgtaggggtctgaaacagtgttgttaccaaagttggccatgggacagaactacacccttacgaactcgtaggggtctgaaaccggataccgagagaaacattgtaccccatagaactacacccttacgaactcgtaggggtctgaaacatagtccgttcttgcacatacccctcaggcgaactacacccttacgaactcgtaggggtctgaaacttttttttgcggccgc

SEQ ID NO: 112 - RNA, 328 nt, synthetic construct
  source 1..328: mol_type = other RNA; note = DNA encoding for non-processed guide and repeat array for 103-7 guides 1,2,3,and 4 targetting eGFP cloned into and expressed by plasmid. (includes restriction digest overhangs)
ccaccttgttgggctataataccctcgtatttgtcggtgttctataacgccagtaaacagttcttcccctttggaaacgctataataccctcgtatttgtcggtgttctataactccccatccaattccactaaaatcggaactgctataataccctcgtatttgtcggtgttctataacctcctgagactgaaaatttatgtccattaagctataataccctcgtatttgtcggtgttctataacgtaatttcccagtagtacaaataaatttgagctataataccctcgtatttgtcggtgttctataacttttttttgcggccgc

SEQ ID NO: 113 - DNA, 328 bp, synthetic construct
  source 1..328: mol_type = other DNA; note = DNA encoding for non-processed guide and repeat array for 103-7 guides 5,6,7,and 8 targetting eGFP cloned into and expressed by plasmid. (includes restriction digest overhangs)
ccaccttgttgggctataataccctcgtatttgtcggtgttctataacgtaatttcccagtagtacaaataaatttgagctataataccctcgtatttgtcggtgttctataacagtgttgttaccaaagttggccatgggacagctataataccctcgtatttgtcggtgttctataaccggataccgagagaaacattgtaccccatagctataataccctcgtatttgtcggtgttctataacatagtccgttcttgcacatacccctcaggcgctataataccctcgtatttgtcggtgttctataacttttttttgcggccgc

SEQ ID NO: 114 - DNA, 30 bp, synthetic construct
  source 1..30: mol_type = other DNA; note = Spacer sequence that targets ueGFP
gccagtaaacagttcttcccctttggaaac

SEQ ID NO: 115 - DNA, 30 bp, synthetic construct
  source 1..30: mol_type = other DNA; note = Spacer sequence that targets ueGFP
tccccatccaattccactaaaatcggaact

SEQ ID NO: 116 - DNA, 30 bp, synthetic construct
  source 1..30: mol_type = other DNA; note = Spacer sequence that targets ueGFP
ctcctgagactgaaaatttatgtccattaa

SEQ ID NO: 117 - DNA, 30 bp, synthetic construct
  source 1..30: mol_type = other DNA; note = Spacer sequence that targets ueGFP
gtaagtttcccatatgtagcgtctccttcc

SEQ ID NO: 118 - DNA, 30 bp, synthetic construct
  source 1..30: mol_type = other DNA; note = Spacer sequence that targets ueGFP
gtaatttcccagtagtacaaataaatttga

SEQ ID NO: 119 - DNA, 30 bp, synthetic construct
  source 1..30: mol_type = other DNA; note = Spacer sequence that targets ueGFP
agtgttgttaccaaagttggccatgggaca

SEQ ID NO: 120 - DNA, 30 bp, synthetic construct
  source 1..30: mol_type = other DNA; note = Spacer sequence that targets ueGFP
cggataccgagagaaacattgtaccccata

SEQ ID NO: 121 - DNA, 30 bp, synthetic construct
  source 1..30: mol_type = other DNA; note = Spacer sequence that targets ueGFP
atagtccgttcttgcacatacccctcaggc

SEQ ID NO: 122 - RNA, 36 nt, synthetic construct; identical to SEQ ID NO: 21
  source 1..36: mol_type = other RNA; note = Repeat sequence identified by the Type VI 103-2 effector protein

SEQ ID NO: 123 - DNA, 36 bp, synthetic construct
  source 1..36: mol_type = other DNA; note = Repeat sequence identified by the Type VI 103-3 effector protein
ctattacactacaccccttttacaggggtctaaaac

SEQ ID NO: 124 - DNA, 36 bp, synthetic construct
  source 1..36: mol_type = other DNA; note = Repeat sequence identified by the Type VI 103-6 effector protein
gaactacacccttacgaactcgtaggggtctgaaac

SEQ ID NO: 125 - DNA, 36 bp, synthetic construct
  source 1..36: mol_type = other DNA; note = Repeat sequence identified by the Type VI 103-7 effector protein
gctataataccctcgtatttgtcggtgttctataac

SEQ ID NO: 126 - DNA, 2787 bp, unidentified
  source 1..2787: mol_type = other DNA; note = MG103-2_codon_optimized; note = Mammalian codon optimized
atgggaggcggaggtagctacccctatgatgtacctgattatgctggcggcggaggcagcattgtaaagaatctgtccaacagcggagaaaagcaagtgctgaagtcaaaagccaagtccatcggggttaaatccacattcgtcctcagcaacaagaaggtaatcataacgtctttcggacggggcaatgcggcgattattgagaaattgatcgacgaaaattcagaagacaagaaaatcatcgctataaaccccaagaacgccttcgacgtgaacttgctcaataaagaacccaaagtgtatgaaatcaagaagggccgggaagaaaccaaaattacgaagcccaatattaaggaaaaggtcggttcagatctgattggggctaaagataaactggagatgaaaatattcgggagaaactatgatgacaatgttgccatacagataatttataacattctggatatatccaaaataatctcaatatatataaattccgcaatcgccactatgaataaccttgctcgctcatctggggtggaagaggtagactatattggagactccgttttcagcgacactgattatgataaaatgattaagcagattaataatgagcggaaaatagacaagtttactccctattacgacaaatgtgagccaagactgacatattttcatgctgtttatgaggacaaaatagatgaaaacaagaagagggtgcctatcgatgcaaagtacctgcacgaaacattgagggtcctgtccgccattcgagtgttctgttttcatgggaagaaggacaacaataattctttgatatttcatttggaagaagggccaaaggaagaggactttccagatgatttaaaggatataatccacaacatatacaagaacgcagtcgactctgctaataagggctttaagaaaagcgcaactaacattaatattctcttcaagattctcgagtgcaatgataataacgaaaagaaggaagtggcacagtcattttataatcacattattattagggatagcaagaaccaaggttttagttataagaaattacgtgaggaactcttgaagaatgaaatttataagaaaataaacgataagaaattcgatcctcaaagaggtaagctgtatacgcttttagattacatcatcttttggtacctcgagaaagaaaagaataccgaccccatgatcattgacaaactttgcaacaaattacggtccatcagaggcactaatgagttcgcgaaggagaagacatacaatgactttgccaatgagatcgctccaaaactcaagaattatatttatcggctgattagctttatgggtgcagaagaaattaagaaattccagaaaatggccaagaagaacgaaaatgagatcgtaagacttgccgaggaaaagattaaggactctgatacgacctacttttctaagctgatatacttcatatcccgctttctggatgggaaagagataaacgaattttatggggcgctcattaataagttcgacaacatcagtgggataattgacgccattaataataaagacttcaactataatgagaaattagagttctccaacctgtatagtcttttcaaaactccccacaaagttgcaaaggagttgcgcatcacgaagaacattgcgtcattgaaagcgattgatccgccggcaatccaccgggaaaagtatatcgacgctgtacttctgttgggattaaaagaaaatgattcaaagggaaaaccttatatagaggaagtagaagtatttgtcgataaactcctggacaagtcaggaaaggacacgaaccttcggaactttcttgtaaataatgtcattagttcaaagaaattcttgtacatcgttaaatattgtaacccgcttacagccaaagagttcctgaagaacacaaatattgtggagttcactatatccaggatggactcaaacttgaaggaacgctactgccaagcaatcggcatttctttcgatgagaataccgccaacagcctcttgactaagaagatcgttgacttatcaggggaagattttctggatgtaaagcagaacccaaagacaaatgacgaaaagctgaagaaggagcaatacagtgcgatcttaggtctgtatctcgccgtcatgtatgtaattattaagtccctcgtgaaggtaaacgcattttacgttatcgcaatttcacgtttagaatttgatacaaagatgattatggggaagatacctaagaagaccctggagaaaactagggacaagaacggaaaaccaatcaacgtcgtaatatatagcgccatcctcaatgatatgcttgaaaaggacaattatctgaagaagaccaaataccggcacattcaacaaaatctgggcgaatactccaatacccttttcatagaataccgtaacaagatcgcccatctctccgtcattaatctgggacataagtatattaatggtgtagaaaagatctactcctacttcgggttgtatcattatattatgcaacgtatattaagggattcccgagaaaagatcaagaattatgagggaagcgatgtggagaataacaccataaacaagtgttttaatgacctcgaaaagtttaattatttctccaaagatttgatcaacatcttaaacctgccctttgcatacaatatcgcaagatacaagaatctcagcatcgaggacctgtttaacagccaagacgagaagtacataccaacagagaaaggcaggaagaagtga

SEQ ID NO: 127 - DNA, 2787 bp, unidentified
  source 1..2787: mol_type = other DNA; note = MG103-3_codon_optimized; note = Mammalian codon optimized
atgggaggcggaggtagctacccctatgatgtacctgattatgctggcggcggaggcagcgccaagaagaaacaatctattcgggacaaaagaaacctggaaaaggagagaaaagcagagaaatttcgcaaaatgtatgatgctggtacggctgaggccgttccagtcccaccgccgaagggtaaacgcaagaagtcactcgtcaaagcggccggtgttaagagcggattcgcggtcggtagtgacgtatatatgacttcatttggtcggggaaacagcgctgtgctggaaaagaagatctccggtagaacagtcgtggatctccctgactccaagggagcattcactgccgaagcggtgtccgatgccgacatcgcggtcacgaatagtcgacttaagtataaggttgcgaatgccaataaccccttgcaccacaatcaaggcgcgccagccgtaaggcaagacaatctgcacgctaagtctaaactggaacgaattttcttcggagaagtttatagcgacgataacattcatattcagctcatatataatattcttgacctggagaaaatcctggcgcaatacgtgttggatgcagtgtatttactccataatacagttaaggactcagataaagacgaccttatggggtatctgagcgtacgtaacagctactacgtatttatgcacgctaatggacaatccgaagacccgatagaaaagcaaattgcagaacaaaaggcccaattcctcaaacggcttaactcaggcaggcttggttacttcggaacggccttttacgaagcccctgcagataagaactctatcgcggacaagcaaaaggatacaaaggaaatataccatatattcgctttgattggctccatgcggcaaagtttgttccatgggcatagaaagaccaagaggcaccaagggtccgcctggatttattgcctcgaggataaactttcacaagaatacaaagacactttagacaagacgtatcgttcagctattgaccgcctcaatagggattttggcaagacaaaccgcgtcaacctgtacattattgaaaacgccttgaagtgcttatacgactctgttgaaccaagtgaactcgcgcaggattactacgacttcattgttaagaaaagctacaaatacctcggattttctatcaaacatatgagggaaactatgctggagaatactgatgctcaaaagtataaatctaagtcctttgaccccgtgagaagcaaactgtataaattgatagactttctgatatatgacctgtactataataggtaccccgaacggattgaggatgtagtatgtcgccttcggctgaccatgtctgaagaggagaagaacgaaatttattgtaaggaagcgaatttcgtatggaagaagttatcagatgtcattctgaatattattggcccagcagttgatgttaataacgtcggcaagataaaacctaataactcaaataagcgctggaatgtggacaaagtgtgtatcaaggccgatactaactacttctgcaaacttatttacgttcttactctgctgctggatgggaaagagatcaacgatctgctcacaaccctcattaataaattcgataacattgccagtttcattgatgtaatggacaaattgggcttaaagtgcgaatttacgcctgagtataaaatgttcgctgattcaaagaaaatatgtgccgatttaaagttaataaatagcttctcacggatgcaaaagcctaacaataaatctatcaaagttatgtttaaggatgcgctggaaattatgggaatggatatcagcgattctgacgagtatattgataatatgcaggatcgagatgaaacaggaaaaccgaagcaacgaggggaccataacttcaggaacttcataataaataacgttattaccagtgatcggtttaaatatttggtaaaatacagtaatgccaaaacggtaagaaaactcgcatcaaacaagaaaatcgttggcttcgttctcaaccacatacccgatacgcaaattgaacgctattactcaagctgtcacgggagcggagcatatgatattaacggcaagagaaagtatctggcagatatactgcatgacctggattataaagattttgaaggggtcattaataatgataatgataagaacgtgacagaccaacagaaagacgataaaagcaagaagcaggcaatcattacgctctactttactgttttatacttggctgttaagaaccttatctacgttaatagcagatacgtaattgcttttcattgtttagagcgagactactatctccatgataagagtctcgacgataactattcaaattgtttcgaactgactgatttcttcattgataacggttatatcaatactcgtgcgagccaatatctgaagcagaacatctcaaatagtaacactttagccgtaaagaaataccggaacaatatcgatcactttgcgatcgtaagggatattggggactatatagagcagatcagagattttacaacctggtttcaattgtatcactactttatgcagcgtatcgtgctcaaggagaatatctccggattcgagaaatataacgatatgatccttgagcataacacctattgcaagaatatggtgaaaagtctgaacgcagctttcggctacaatctggtaagatttaaagctctcagtattgatgaattgtttgataggaacaattatctgcctgataagacagagaagaattga

SEQ ID NO: 128 - DNA, 2994 bp, unidentified
  source 1..2994: mol_type = other DNA; note = Mammalian codon optimized; note = MG103-6_codon_optimized
atgggaggcggaggtagctacccctatgatgtacctgattatgctggcggcggaggcagcaagaagaaaacatccatccgggaccaacgagaacaacaagccgcgaagatgagactggcccaccaacaaaaggaagcagttcgccaggaaacggtcagagcccagcgagaagctgaggaagcagccgcgcgcgagaaggagcggatcaggcaagcggcacagaaagctgtccgtctgaaagaactcaataacgatcctaactatcacccaattagacaacgaagcctcagcgaagaaaagaagtccaccgctaaagctgcagggttaaaatctacctttatcatgggtgagaaggaccttctcatgacgagctttgggaaaggcaatgccgcggtcgtggaaaagaaaatagcggcgggcgaggttagcgatttgacagataaacctgcattcacagctgtggccgagaaggacatatcatttcggatcgatggtagggtgaagaacgcatccaccgacaacccactccacatagccgaagaaaagacaaagacacgggaagacttgattcacgcccggagctcccttgagatgctgtattatggcgagaattttgaagacaacatccacatccaagcgatttacagcatcttagatgtggaaaagatactgtcaatccacatcaataacattgtgtatatgctcaacaatcttctccgggaagagaaagaagatctgatggacttaataggatacttgcggactaatgataaaccatacgaaaagttccttgagggtcagggcaatgacggagagctgttgaccgcttttaggaacctctgtatgaataaacgtctcggctaccttaatcttgaggtaattcctaaagagccacccaagaacaaagatgggaagcctattcgaagaaaagccgacccaaatacaattaaaatgactgatgaagaattttatcacattctggttgccctggggaaaatgcgcaacatgctggcgcacggaaatccaaaacaaaacatatacaagattgatggtttcggtaagaatgatgccataagtgcggttctcgaccggctgtatcaagagcgtataatagaactgaacaaggggtttctcgagaaggctggcaagaacctcgcaatccttttccacgccttttccgtcaaagagcgtgaggagaaggctaaatatgtgcaagattattatgattttaccgttcgcaagaattataagaataccggtttcagtatcaagctcttgagggaacacatgactaccgacgtcgaagaggcctttgtccttcgcaacaaacagtatgattccgtacgcggaaagttgtatccgttcattgactttgcagtttacagatactacagacagcatcccgctgaagccgaggaactcgttggctctttgcgggcttctatggacgaaaaggagaaggatgtcatatactcaagagaggctgaaagaatctgggagaaagaccaagtcggcgatctcattttgcaccatattctcccagaaatgagcggcgacactataaagaacctgaaacccgataaggacgtcactgaagaaatgctgcaaagtgttcttataaccgatgaggctactgactttagcaaaatggtctacatgcttacattattcattaatgggaaggagattaacgatcttctcaccacgcttattcatcagtttgagaacatagatgcctttacttcagtgatgaaatcacagaacctgcgaaccgggctcgtgaaagacttccaacttttcaataggtccgcgcaggttgcaaaagaactgcgggtaataaacagctttgcccggatgactaaagaggccgccttcgcaaaggaacctatgtatattgaagcattccgcgtgcttggcatgaaggccgacgagaagcaactcgtgcaggaagccaaagatttactcgatcctgagaaaagcggcgcaggcactcagaggcgcggggtgagaaatttcatagcaaataacgtcatcgaatcagtgaggttcaagtatcttattcgctatggcaatgtcaacaagctgaaggccattgcccaatcccctgccgtggtttcattcgtcttaaaagatatacctgatgatcaaatacgacggtattgccaagacttgggagatcgtcgtgaaatggatgtgactgcaatgagatcaatactgacggaaagattaactggatttagctttgaggatatcagggatgtcagacaaaacgatcagaaagcaaatcagaaagagcaacaagagaaacaacaaaagcaagcccttgtgcggttgtacttaacggtactctatctggccctgaagaatttggtttatatcaattcacgttatttcttggcatttcattgtgtggagcgtgatcgattggtactcgactatgaatattggaaagccacgctgaatgatgagtatagaaacaagccaggatatgaattaagtgcatttgcaaaagagttcctggaaaagtaccctcagaagaagcgcgtcaaggcgtatcttgaactgaacttcgccaacagcgatgattgggctatcagcgcctttcgcaataaagttgagcatttggacgcgataaggaatgcagataggtacattgctgacgtcaaagagataagaagttggttcgaactttatcactatataatgcaacggagaattatggatcagtacgagtgggatagcactcacgagtctaggaaagaaaagggcactatgataatttctgaagacaggttgaaccccaagactcttgagtacttcaacaaggtgagaacttatgggtcatgttgtaaagatttcgtgaaggcgttgaacgttcctttcgcatacaaccttccacgttacaagaacctgagcgtcgatgagctgtttgatcgaaatcgacccttggctaaggggaaaggcatgggaaggaacctggagaacgatgaatga

SEQ ID NO: 129 - DNA, 2913 bp, unidentified
  source 1..2913: mol_type = other DNA; note = Mammalian codon optimized; note = MG103-7_codon_optimized
atgggaggcggaggtagctacccctatgatgtacctgattatgctggcggcggaggcagcattaccaagactattgagagcggatttattatgggtgatgtaaagacaaccgagaagaagtccatcgccaaggcaaatggcctcaagagcactttctttattgacaatgataaaaccctgatgacatccttcggaaagggcaatagtgcggtcctggaaaagatgattgataactacgatgtagaagaattgcaggatgaaaataagaaggcattcgacgctgcagtgaaaacgctcggagaagcacacatcaaaggcaggtgcggtgaaagtgatgtagtccttcccaaatcctctgacacgaaccatttgcaagcaaaatctacaattgagaaaatatactttggaaagacgttcaccgataacatccacattcagatcgcttacaatataatggacatcaagaagatcttcgcaatctacgctaatacgatagtcgagagcattgataacctgaaagataggcatacagattctgtcgctgataaagatgagttcctgggaatgttgtatacctttaattcagtggaaaagagttctctcgctagtctgatacttaaagaggggttaatcgacgaggaaaagagtaaaagctatttctttataaagggaaacgtgtggaaaagtaagaagcacaaacaactcaaagctctcaacgctaagttcaattatcatggagactttcagaacgaattgcgtagtcttgggacctatctccagaatcaattccgagatttaaagaaaatgaagaaggcagctaagtcatatgacgacatacaaaagatcattaaaagcagcatttacttcggcgatgccttcttggatcagaatggtaatgttgacgtgaatactgcttatacaattatatgcctgctcggagtattacggcaacaatcttttcatgaaaagagtaccactaacacttggatctttgagatcgataacaaacgggaccaaggatttaacaatgcccttgatacagtattcaagaataaggtggataagttgaataacgatttcttgaagaactccaaggtaaacctgtccgtacttttcgaaatttatggtggagaaaagaaaagcgaacttgtcaaggaatattacgacttcagcgtcaggaaatcttataagaatatgggtttcagcataaagacactgagagaaactatccttacattcgaggatgcaaaagtgattacagaccaaaagtatgactccgtaagatcaaagttctacagtctgtttgattttgtcatttatcgttattatctcgccaatcaaaagcgtggagaggatttcgttgataagcttagaaagtgtgtcactaaggaagacaaacccaacctgtatgaaaacgaagcaaagatgctgtggacgaacatcaaaacctcagtgctcaaacaaatcatgcctaaaatggacggtaattatataaagaagatcgcgaagaatgaggatgttgcctcacaatactccagtgcactcgatggcattatgcaaacggcagaaaatacgagctatttctctaagacaatgtactgtgtgagcttgttcctcgatggaaaagaaataaattgtctcctgaccagtctgataaacaaattcgacaacataaattctttatgtagggtgctggtcgatgctggctacgaaagtgatagcaacattttcaccgataagtacatgcttttcgctgatagtgagaaaatcacggacgaactccggttcgtcaattcctttgcgaggatgtctaaaacgactaaagctgtcaaagatgggaacgcttccgtaaataggcaaatgtattatgattctgctgcattgtttggcgagttcaataaagagaaagtggaaaagttgtatcacctgggcgacgtcaaggccaccaagtttgataaggcgttgcgcaactttatgatcaacaacgtgattaacagtaatcgattcaactacgtaactaggttcataaacccagctgaaggcgcgaggattatgcagagcaaagttctggtggagtttgcattgaaggatatacctgaagctcaattgatccggtactgtaaaactgcagggattacctttgacgccgataatcctgattgcgccaaaatggcaaaagatctggcagacatgctgaacaaggtacgattcgataacttcgttaaagttaaacaaatagtcaaggccgactccgaagaggccaaacaaaaggagcgctataaagccatcatcaggctctatctgacagtcctttatctgattgtgaaatccctcgtgaaagtcaacatgtcctactccatcgcgtttggcattttagagagagactgcgagattatgaatcagaaatacgaatgcaaggattacttcatccggagtgaaccacattcaggtcaggttaggggatacgatttcgcagccaccaaaattactgatgacttccgaaaggatggacggctcaacaaacgagtagcaaattccattgagacgaacaaagagctgtacaaagatgatctgttctacgcttatcgcaaccaagtggcccatctgaacgtaatagagactctccctaattacgtcgacaggataaagaatgtcaagtcatattttgatctgtatcattgtattctgttcctgaaccttatggagaataaagccccctatttgcaagaaagcattaacacggtgaagaaccatgtcagttcaaatggaaagactgtctacgaaaacgtgtgtgagcataatacggtcagcaaggattttctgtatggtttgaacacgcctttcgcgtacaacgctgcacgatacataaatctttccaatagagagaaatttattgaagggtacggaaaatga

SEQ ID NO: 130 - DNA, 2949 bp, unidentified
  source 1..2949: mol_type = other DNA; note = MG103-9_codon_optimized; note = Mammalian codon optimized
atgtacccctatgatgtacctgattatgctgggtctggtggttctaagaagaagctcaatggcaacggcgcgatcgggtctgcatcactgaagaaagcgtttcaacagagacagaatgcacagagtaaagccatcttagctcaacaagaggaagagaaccgtaagaaggaactgcgggagcagcaagcacgcgaggctgctgctgccgctgaggctttgcgacgaaaggagctcgagaagaatcttcatcccgtccgcgaacgcaagctctcaactgagaagaaatctagcgccaaagccgcagggttaaaatctaccttcatcctgagcgataatgaactgcttatgactgcctttggccagggcaacgaagccatcattgagaagaagatcacagatgggcgggtcgaaaatctgaatgagaaacccgccttcacggcggtaccagaagaaaagaaagcctttcgcattgacgggagagtgaaaggtgcccagaccgataaccccctttacaccgcagatgccagtcttaggaagcccgacgatcttatacatgctaaagccgccctggaaaagcgctattttggccaaactttcgactcaaatttgcatatacaggccatctacagcatcttggacattgagaaaatcctggctattcatgtgaataatatcgtgtatacattgaacaacttccttagacaggaaggcgaggacgtggacgactttattggctacgtgacttccggcgaaaagacttaccagcaatttatggaatctaaggacaataacgccgatgagctccgcgccctcttcactcggctctgttccgccaggcaactgcgttatctgtctcttgagatcatgaagcccaggaaccccggcgataataccaaggaagacgagaatacgatcaaactgacggaagaggaattttataatattctgtacgccatgggcattatgcggcaaatgcttgcgcatggagatccatacaagaacatttacaccctcgaaggatacggaaagaacgccagtgtagagaaggtcctgacccggctttataaggaacgtataaatcagctgaataccgattttctgagcaaggccggcactaacctcacattgttgtttaaggcctttgggataaccgatgtggagaagaagggctgcctcgtgcgcgattactatgacttcactgtcagaaagcaatataagaacctgggctttagcattaagctgctgcgcgagcatatgacagcagatatcgaggaagccttcatacttcgggataaggcatacgacagcgtgaggggcaagttgtaccctttcatcgactttgccctgttccgatattaccacacccacaacgaggagactaatagcctggtgaatggcctgagagccagcttcaacgaagtggaaaaggacgggctttatgcgcgtgaggctgcccgaatatggcccaaagtgcggagtgtgattctggaacacatactcccggagatgtctggcgaggtcataatcgaggccaaatccaactcagacaaagatattaaagagaccatgttgagcggagcgatgatatcaaccgaagctacggacttttccaaatttatttatctgaccaccctgtttataaatgggaaagaaatcaacgacctgctgactaccctgattcacaaatttgagtgtatcttctcttttctggccgtgatacagtcccagcatctccctagccgtttcgtcgaccagttttctgtctttagcgaatccgataagatcgctcaggaactccgtgcaatcaacagtttcgcccggatgtctcggcccagcgccaaggccaaggagattatgtatattgaagcctttcaggtcctgggtatgcgcgccaactccgatcaattggagagagaagcgaaagagctgctggaccctaaaatcagtggcgaaagatcacaaaagcgtggaataagaaatttcatttccaagaatgttatcgcgagcgataggttcaagtacttagtcagatacggcaacgtaaagaaattgaagggcattgctacgaatcgcgctgtggtaagtttcgtgttgcaagatattcctgacagccagatcgcccggtactacaactctgttacgggcgaggataaggactttcaacctgcgatgcgaaattttctggctgatcgcctgactggtttctctttcgaggacatcagcgatgtaaggcagaacgaccaaagagccaaccagcgccaacaagaagagaaacgccagaagcaggccctcgtgcggctgtatctcacagtgctctacctcaccctcaagaacctggtgtacgtcaacagccgctacttcctggccttccactgcatggagagggaccgccggctgcttcaccccgaaaagtgggagaaaatcccccgagagaaacagtttgacccagaatttggtgcaagtgtcttcgctcgggacttcctggctgaatatccccgcaaaactcggcaaatgacctatctgaatcagaattttgataacagtgatgaatgggcactgagagcctttcgtaacaaagccgagcatttggatgcagtgcggaacgccgacttgtatctgggcgacattcgcgagttccacagctggttcgaactttatcactacattatgcagaggcggataatggaccagtttacctttgattctacccacaagacccgagacaagacaaggatgattataagcgaagaggagctcaaccctaagacgcgggagtacttccatcttatcgcacggtacagggtatactgcaaagattttgtgaaggcactctgtgtgccattcgcatataacttaccacgttttaagaatctctcaatcgacgggttatttgacaagaatcgccccggtaaagagaaagacggggaagaaactttacctgaggattga

SEQ ID NO: 131 - DNA, 2841 bp, unidentified
  source 1..2841: mol_type = other DNA; note = Mammalian codon optimized; note = MG103-10_codon_optimized
atgtacccctatgatgtacctgattatgctgggtctggtggttctaagaagaaagaaaacgccaagcaaaggagagacagagccaaagcaatgcgcaggtcaacagtagaagccgccgagaccaaacggaatgaggaacggcgcctggcagaggagatacatattgttgaggaagaggtgcggaagaaacagaaggaagaggagaagcggctgcgggccatcaaggcggccgagaaggccgacatctcactggccaagaagactaaaacatctgccaaggccgcgggcctgaaatctactttcgttatcgacgataagcgccttctgatgactagcttcggcaaatgcaatcgcgcaatagctgacaagtacatagatgactctgacataaaggatgcaggcgaggaagcaagtctcagggtggagagaaagggcaagaattttcaagtgtccggccggaaagttaaagacgccacagccgataaccctctgcgttctaagaagccggccgggcaggaccagatattctgcaaagaccaattagagcacatctattttggtaccagttacgtcgacaacattcatattcaactcatctacaacatactggacatcgagaaaatcctggcggtgcaagtgaacaatattgtatatgagttaaataacattctgcgagaagacaaggccgagtacgacgatctgatcggctacatgtcttgcgactacacctttgagcgctttaagaaacataacaatgggaagctctatgaaaagttcaggaagctgataggccagccccaactcgggtatttcggcaaaacatttataccatacgacaaggcaggaaagaagctcagtagtaatgctatgcctgcatttgaggagaaatgctataacttgctgtctgtgatcggcaccgttcggcaagccaccgctcacgggagtgagcgcaccagagcaaagttataccaattcgagaaagaatttgacggagagaataaggggtgccgaatccaagcgcggcgggctctggaaagtctgtatagggagcgcgtgtgctcactcaactcaggatttctggacatgagcgggaaagacctctacatactgttcaatgccttcgacgccttcgacaacgagaagaagaaacaaatcgtccgggagtattacaacttcctgattctcaaggcttacaagaacatgggtttcagcattaagaagctgagggaaaagatgattgcccgctgtgctccagagctggccagtgacgattttcggacggtaaggcaaaagctctacaggtttgttgatttcgctatatacagattttaccttaatgacgaaaatagggcttacactgaagagttcgtggcttccctgcgcgcttctgtgaaggcctcagagaaagaagccatgtaccaactggaagctgagagactgtggaaagaattaaagagcatcatcaatgaaagaattacgacgtggatggatggggaacgtattgctaaaatcaagaagcccgagatcgataagcatgtgatagacgacatagctattcctgagtctgctaaccctttctgtgagatgatttatctgctcactatctttcttgacggcaaggagattaacgacatgcttacccagctgattagccgctttgagaacatcgccggatttattgatgtgttaaagggcgaaggcatggaccgagcgtttgctccgccctatgggctgttcgacagttccgacaaaatcgccgaggagcttcggataattaacagttttgccaggatgaccaaagaagacccgagcgcaaagcccataatgtacaaggaagccgcccaggttttaggtttcggaaacgaagaagagctcgaagactatgtgaatgacatgctggacaaagatcacaagggcggaatggggttccggaactttatcgccaacaatgtcatagaaagcagtagatttaagtatttggttcggtacggaaacccaaagaaagtacgcgctattgccgaaaataggaaggtcgttgattttgtgctgaaggagatccccgatccacaaatccaggcttactactcttcctgcacaagcgggacaacaggtaattatgacgagatgcgcggcgccttagcgactataattactggcctgcacttcaaagactttgaggacgttgtgcaaggcaataatgcctctatacaacagaaacgggataaagaaaggaagaagaacattattcgtttgtacctgaccgtactttatctcgcccagaagaatttgatctatgttaattctcgctattttctggcatttcattgcgcagagcgggacgccatgacctacgattcaaaccgattcaacaaagatgcactgtgggaagacagggccgcatttgctaaggcattcattgcagagcaccccaagaagaaacgtgtgcaactttatctttataagaacatttccaattctgacgtctggtccttccgcgcttacagaaacctcgcagagcacttaggcgctattcgtaatatggacaagtatattgaggacatacgcggatttgagagttattttgagctctaccactacttgatgcaaagaagcatcatcgacagatttagatacgaaggaaggacagagagcggggccgagggtaaacttctggaatatttcgagttggtggaaaagtacaatacttattgcaaagacttcgtgaaagctctgaacataccattctcatacaatctggcccgctataagaacttatccgtgaatgagttgttcgaccgcaataattatttgcccaacaaagagcgagagaaagatgagtga

SEQ ID NO: 132 - DNA, 2772 bp, unidentified
  source 1..2772: mol_type = other DNA; note = Mammalian codon optimized; note = MG103-12_codon_optimized
atgtacccctatgatgtacctgattatgctgggtctggtggttctatagtgaagaacctgagcaacagcggcgagaaacaagtcctgaagagtaaggcaaagtccatcggggttaagagcacatttgtgctctccaataagaaggtcatcatcacgagctttggcaggggtaatgcagccatcattgagaagctcattgacgaaaactctgaggataagaagatcatcgccataaatcccaagaatgcgtttgatgtcaacctcttgaacaaagaaccaaaagtctatgagatcaagaagggtcgcgaggagaccaagattacaaaacccaacattaaagagaaagttgggtccgacctcataggagccaaggacaaactcgaaatgaagattttcggacgcaattacgacgacaacgtggcgatccaaatcatttataacattctggacatctcaaagatcatctcaatttatattaactctgctattgccacaatgaacaatttggcccgctcaagtggagtggaagaagtcgactatattggcgacagtgtgttttccgacaccgactatgacaagatgataaaacaaattaacaacgaaagaaagattgacaagttcacaccctattatgacaagtgtgagcctcgactgacttacttccacgctgtgtatgaggataagatcgacgaaaataagaagagggtccctatcgatgccaagtaccttcatgagacgctgcgcgtgctgtcagcaatccgcgtcttttgttttcacggaaagaaagacaataacaacagtctcattttccatttggaagaggggcctaaagaagaggatttccctgacgaccttaaggacattattcataatatttataagaacgctgtggatagcgcgaacaaaggttttaagaaaagcgcaaccaacataaacatcctcttcaagatcctggagtgcaatgacaataatgagaagaaggaagtagctcaatctttctataaccacataataattcgcgacagtaagaatcagggcttcagctacaagaaacttcgggaagagcttctgaagaatgaaatatataagaagattaacgacaagaagtttgacccccaacggggcaagttatacaccttactggactatatcatattttggtatctcgagaaagaaaagaacaccgaccctatgatcattgacaaactctgtaacaaactccggtctatccgcgggacaaacgaatttgcgaaagagaaaacttataatgacttcgccaatgaaatcgcacctaaacttaagaactacatctacaggcttatctccttcatgggcgcagaggaaataaagaaatttcaaaagatggccaagaagaatgaaaacgaaatcgtcaggctcgcagaggaaaagatcaaggactctgataccacgtactttagtaagctgatttactttatctcaagatttctcgacggaaaagaaattaatgagttttacggcgccctgatcaacaagttcgacaatatatccggcatcatcgacgctattaacaacaaagactttaactacaacgagaagctggagttctccaacctgtacagcctcttcaagacgccacataaagtagccaaagagctgcggattaccaagaacatagcatctttgaaagccattgatccgcccgccattcaccgggagaaatacatcgatgccgtcctgctgctgggtcttaaagaaaatgactccaagggcaagccttacattgaagaggtggaagtcttcgtggataaactgctcgacaagagtggcaaagatactaatcttcgcaattttctggtgaacaacgtgatttccagtaagaaatttctgtacatagtcaagtactgcaaccccttgacagcaaaggagtttctcaagaatactaatatcgtggaatttaccattagccgcatggactccaatctgaaggagcgctattgtcaggcaatcgggatttcctttgacgaaaatacagcgaactccctgctgaccaagaagatcgtggatctctctggggaagattttctggacgttaagcaaaatcccaaaacgaacgacgaaaagctcaagaaagaacaatactccgcaatcctgggcctgtacctggccgtcatgtatgtgatcattaaaagtctggtaaaggtcaatgccttctatgttatcgctatctcccggttagaatttgacaccaagatgattatgggcaaaatacccaagaagacactcgagaaaacgagggacaagaacggaaaacctataaatgttgtgatttacagcgccatactcaacgacatgctggagaaggataactatttaaagaaaaccaagtatagacacattcagcagaacctgggcgagtattctaataccctgttcatcgaatatcgcaataaaatcgcgcatttaagcgtcattaacttgggacacaagtacataaacggggtagagaaaatatacagctactttgggttgtatcactatataatgcagcggatactgcgagactctagggagaaaattaagaattacgagggcagcgacgtagagaataacacaatcaataaatgctttaacgacctggaaaagtttaattatttctccaaggacctgatcaacatactgaacttgccattcgcctacaacattgcacgctacaagaatctttccatcgaagatcttttcaattctcaggacgagaaatacatccctactgaaaagggccgcaagaagtga

SEQ ID NO: 133 - DNA, 3003 bp, unidentified
  source 1..3003: mol_type = other DNA; note = MG103-12_codon_optimized; note = Mammalian codon optimized
atgtacccctatgatgtacctgattatgctgggtctggtggttctaagaaaggaagtagcattaaggaacaacgcgaaagggagaaagcacagcgcatgagtgcctacaagcagagggacgcagagcgaaaagaggcagtggcattaagtgagaatgaaaagaagcaggctacagctcagaaggcgtcaggggaagacaacaaagctgaaaagaaaagcacggcgaaagccgccgggctgaaatccacctttgttatcaaaggggatgagctgctgatgacaagttttggtaaaggaaatgacgccattatcgagaaacggattcgcaacgccgtggtcgaggatgtaagtgaaaatcctattctgtctgtgtttccagagaacgaaaggtacttccgcatcgacggaagggtgagcggtgctaccacagataatccactgcactccgccggcgaggagaaggagaagaccgacttaattcacgccaagaacgcgttggagaaaagattcttcgggagcaacttcgaagacaatatccatattcagatgatctacagtatcttggatgtggagaaaattatggcgccccacataaacaacatcgtatgtcagttaaacaatatgttgcgtaacaaaggatcagacaatgatgacctgatagggtacctgagcttggaaagggagaaatcatttgaaaagatcaaagcaaacggaagtgcaccagttaaagagggagagaagctgtcagcagcccagtggaagtatatgttggtgcgtcgtctcgcaaggaatggcaccatgggttactataatctcgaagttctcagggacgaacgagaacaagaggagtggaatagaatccaagaagagcggaaacaaaagtgtgaggttgaaaatcgcgaatacaaggcccccgaagaagaccgtaatacaattaagctgtcttatgaagagttttaccagactctctgcctgttaggcgagatcagacaaagcctggcacacggtaatgagcctagttttctgtatcgcatcgaggactttgacaagaagtttccgcagagtgccaaagttacagttcaacggctgtaccatgatcgggttcaagaactgaatcggaaatttctcaaaatggcgaagaccgatctcatcctgctgttccgggcctttggtgttcagacattagaggagaagaaagagtatgtccaggcttactatgacttcattgtgcgcaagcaatacaagaacctcgggttctcaattaagaaactgcgggagagcatgatcgaggtcatccccgaagccatggaagctggggatgataaatactctaccgtcagaaataagctctatcgcttcctggattttgccatctcccaacacttcattaagaaagaagaggcacaagagaacctcgttggccagttgcgggccagcaggaccgacgaggagaagaccgagatctacgttaagaacgcaaaggagctgtggccccagatcagggagctggtaatcaaacacatactgccacaaatgaaagtgaccgaagtggcaaaggtgatcgaggaaaataagaaaggtgataaggacgtaaccgaacagatgctcgagggtaggtggataagcgattccgcaacattcttcaccacaattatctacacgttgaccttattccttgacggcaaagagatcaacgaccttattacacagctcattcataagtttgagagcataagtgcgtttgtcgaggtgatgaggtacgaaggtatgccattctccgtcagcgggaaatatgacgtcttcaatcaatcccgggaagtcgccgctgagttacgggtgttgaacaacatagcaaggatgaccagaccagctgctcacaccaagcctgtgatgttcaaagacgccctgaaggttttagacatgcaaatggacaattcagagatagacagttatgtctctacccttctggaccctgtgctctctgcggcagggtcacataatcggggaataagaaacttcattgcgaataatgtcatagaatccagccgctttcactatattataaggtacggtaatgtcaacaaggtgaaaggcctcattaataaccccagggtcgtgaggtttgtcctgaaagacataccagataaccaaatacatcgctattatcgtaccgtcacggggcaagacgtcagcttcgataattcaatgcgacgctacctcgcagatcgcctgacgagctttagctttgtggagttgagggatgtccgccaatctgatagcggagcttctgccgccgataaggcggaaaaggaacaaaagcgcgccatggtgtctttatatcttacaatcctctacctcatcataaagaacctggtttatataaattcccgatatttcctcgccttccactgtgtcgagagggaccgtctgctcttggacgaagagcactggggaaagatggggcaggcgaaccaggatgaccctgattacagttacaaggcctttgcccgcgatcagttagcaaagtacccacagaagccgaaagtacagcgttacatggcgcaaaattttgctaattcagacgactgggccatcagagcctatagaaataagatcgagcatatggtggccgtgcgcaatgccgacagatacctcaatgatattcgtgagtttcactcttggtatgagctttatcactacacaatgcaacgtgagatccgcgcccagtatgaatgggagtctgcgagagaatcaagccgcacacctggccgcatgattgtggagaaggaagaccttaatcagaagacgtttgagtacttcgacaaggtagagaagtacggcacttgctgccgcgattttattaaagccctgaacgtcccattcgcctacaaccttccgcgttataagaatctgtccatcgacggattattcgataagaatagacctggcaagaagggtgaaggaaagaaacttgaacttgacgagaagacctacgaaaactga

SEQ ID NO: 134 - DNA, 2862 bp, unidentified
  source 1..2862: mol_type = other DNA; note = MG103-14_codon_optimized; note = Mammalian codon optimized
atgtacccctatgatgtacctgattatgctgggtctggtggttctaacggggccaaatcaaagaaatcagtcgccaaagcaaacggcctcaagagtaccttcaagctgggagagaataaatactttatgacttcattcggaaaaggcaatactgcgctggaagagaaagacgtggaagaaagcaccataacagacctgcggaacacctttaaggccgaatttgccgataatgataataccaccatcgtcatcaatggggtcgccggctccgctggagtccagttaccctgcaatgacgccaatcaactcagggccaagagcaccatcgagaaaattgtgtttggggagacctatagtgataatatgcacattcagatagcctacagtgtcatgaacattaagaagctgctggctgtgtatgccaataatatcatctataccgtgaataatctcttgaaaactgactataactacgactttctcgggatgttccctacaatgaatgattttcgcaagggctcaattgcctataacattattaagacgcaactcattaacgcagagcgctctggtatcacgccaaccaatgggacgccccgtcgcggcacctattttatcaacaaagacagttggaaagccaaccgaaagacggctcttaaagagcttgaactgttggttaaccaggagcctgacagcggaatagacttctggggtaagctctcttactgcatcatacatcgcctggggttcgggaattacgaacacataaagaaggccgcagaaagtttcgaaaagattaagaacatagggctctgccagtattacttcagtgatgtcttcacagacaagaagagcgagccaaacatggagtatatatatgaatcccttaggctcctggggatcatgagacaaaccgctttccattctagtgagattaagaagactgggaataagaatagttacacactgttcgagatcgattgttgcggggagaaagagatgaagtcaaccctggacaacataactaagaagaaaatcgacgcccttaatgacaagttcatcaacaccagcagcgtcaatcttaatatcctgttctctgcgtacccgaaagccgacaaatctgaaataatcaaggaatattatgacttctccgtgaggaaggtgtataagaatatggggttttccgtaaaagcgctccgcgaaacccttttggctaccgaggaaagtattaagaatctgacttccaaggactttgactctgtgagaagtaagctgtactctctcatcgatttcgtgatttactgtcactacaagaataacgaagacgaggctgagtcttttgtgtccgcactgcgctccctcatatctaccaaagaggacgaaaagtccggggttacctacgacgacaagaagaacgaactgtatatcaaggagtccaagaggttatggaagtccatcgaccggacagtggttgaacacatcataccagacgtcgagaagtgtaagaagagtgaaaacatcaagcctctcctcagtgatattgaacggaaagtaaaagagtccattcgcgacatcatccagcactatgacgaccttagctatttctccaaggcagtatattgtatatgttctttcctggacggcaaggaaataaatatgttcctcagcagcctcattcaggactttgagaacattgagtcctttaaaagagcgatcgaatacaccgataatgacatcagcttttccaagaactttgacttctacaatcattgtcagaatatcgcagaagagctctctttcatcaagagcatcgctcgcatgaacaagagcaatatcgcagtcaagcagtcaacggttaagattaagagcttccaatacttcgagagcgccgcactgttcggggagaataaccaagagacggtcaaagaattgtttaagttggagtccggcaagtccccacagaaaggagcccatgtgctgcggaacttcttcatcaataacgtgatcaactccaataagtacagatatgtgatcagattcgtgaacccctctaacgcacgcaagataatgcagaacgaggcgattgtgtcctttgctattaaagaggtcgacgatagccagatcatccggtactgcaactctatcggcatcgcttatgacgctgagaatcccgacctggacaaaatgagaaatgagctctccaagaagctcatgaaggtgaattttgagacattcgccaaagtgagcaatgaccgggataagaatatcgaaaaggagcagctgaaggccttaattggcctctacctggccataatataccttattacgaaatcactggtcagaataaacaccagctattccatagccttcagcgtattcgaacgagactgttacattatgtccgagaagtctaaggataaggaagacgagttttcagcgctgctggagaatgtctctgtcaagaacaatcctcagaatatcaccgagtactttaaacgtagcggtttgctcaataagagagtccgtaatttaatcaacaggaatgagaagcatttcaacgcagaggtgttcaaatactttcggaataacgtggaacacctcagcgtcgtgagtgccctgaccaattatgctgaggagatcaaacgggtggagtcttattttgatttatatcactacctgatgctcaaaattataagccgcacttatgtggggaagtacattgaaaatatcgatagcgccctggatcaccaaacggcctacaaggactttctgtacggcattctcactccttttgcatataaccctgcccgctacctgaacctgacatctaaaactctgtttattagaggatatggcaagtga

SEQ ID NO: 135 - DNA, 340 bp, synthetic construct
  source 1..340: mol_type = other DNA; note = Chemically synthesized guide array with guides 1 and 2 for 103-9
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancncncncntnancngngnanantngngntnangngngngntncntnanananancngncncnangntnananancnangntntncntntncncncncntntntngngnananancngnanancntnancnancncncncntnancngngnanantngngntnangngngngntncntnanananancntncncncncnantncncnanantntncncnancntnanananantncngngnanancntngnanancntnancnancncncncntnancngngnanantngngntnangngngngntncntnanananancntntttt

SEQ ID NO: 136 - RNA, 340 nt, synthetic construct
  source 1..340: mol_type = other RNA; note = Chemically synthesized guide array with guides 1 and 2 for 103-10
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 339: mod_base = um
  modified_base 338: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancntncncntngnancntngntntngntncngngngnangntncntnanananancngncncnangntnananancnangntntncntntncncncncntntntngngnananancngnanancntnancnancntncncntngnancntngntntngntncngngngnangntncntnanananancntncncncncnantncncnanantntncncnancntnanananantncngngnanancntngnanancntnancnancntncncntngnancntngntntngntncngngngnangntncntnanananancntntttt

SEQ ID NO: 137 - RNA, 340 nt, synthetic construct
  source 1..340: mol_type = other RNA; note = Chemically synthesized guide array with guides 1 and 2 for 103-11
  modified_base 1: mod_base = gm
  modified_base 2: mod_base = gm
  modified_base 3: mod_base = um
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
ggtncnanantnancncncntntncntnanananantntnangnanangngngntncntnanananancngncncnangntnananancnangntntncntntncncncncntntntngngnananancngngntncnanantnancncncntntncntnanananantntnangnanangngngntncntnanananancntncncncncnantncncnanantntncncnancntnanananantncngngnanancntngngntncnanantnancncncntntncntnanananantntnangnanangngngntncntnanananancntntttt

SEQ ID NO: 138 - RNA, 340 nt, synthetic construct
  source 1..340: mol_type = other RNA; note = Chemically synthesized guide array with guides 1 and 2 for 103-12
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancncncntntnancncngnantntngngntnanangngngntncntngnananancngncncnangntnananancnangntntncntntncncncncntntntngngnananancngnanancntnancnancncncntntnancncngnantntngngntnanangngngntncntngnananancntncncncncnantncncnanantntncncnancntnanananantncngngnanancntngnanancntnancnancncncntntnancncngnantntngngntnanangngngntncntngnananancntntttt

SEQ ID NO: 139 - RNA, 340 nt, synthetic construct
  source 1..340: mol_type = other RNA; note = Chemically synthesized guide array with guides 1 and 2 for 103-14
  modified_base 1: mod_base = gm
  modified_base 2: mod_base = um
  modified_base 3: mod_base = um
  modified_base 338: mod_base = um
  modified_base 339: mod_base = um
  modified_base 340: mod_base = um
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
  modified_base 1^2: mod_base = OTHER; note = phosphorothioate bond
gttntntnangnanancncnantngncnanananantncnancnancnancncntncntnanananancngncncnangntnananancnangntntncntntncncncncntntntngngnananancngntntntntnangnanancncnantngncnanananantncnancnancnancncntncntnanananancntncncncncnantncncnanantntncncnancntnanananantncngngnanancntngntntntntnangnanancncnantngncnanananantncnancnancnancncntncntnanananancntntttt

SEQ ID NO: 140 - RNA, 340 nt, synthetic construct
  source 1..340: mol_type = other RNA; note = Chemically synthesized guide array with guides 3 and 4 for 103-9
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 339^340: mod_base = OTHER; note = phosphorothioate bond
  modified_base 338^339: mod_base = OTHER; note = phosphorothioate bond
gaancntnancnancncncncntnancngngnanantngngntnangngngngntncntnanananancncntncncntngnangnancntngnanananantntntnantngntncncnantntnanangnanancntnancnancncncncntnancngngnanantngngntnangngngngntncntnanananancngntnanangntntntncncncnantnantngntnangncngntncntncncntntncncngnanancntnancnancncncncntnancngngnanantngngntnangngngngntncntnanananancntntttt

SEQ ID NO: 141 - RNA, 340 nt, synthetic construct
  source 1..340: mol_type = other RNA; note = Chemically synthesized guide array with guides 3 and 4 for 103-10
  modified_base 1: mod_base = gm
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
  modified_base 2: mod_base = OTHER; note = 2'-O-Methyladenosine
gaancntnancnancntncncntngnancntngntntngntncngngngnangntncntnanananancncntncncntngnangnancntngnanananantntntnantngntncncnantntnanangnanancntnancnancntncncntngnancntngntntngntncngngngnangntncntnanananancngntnanangntntntncncncnantnantngntnangncngntncntncncntntncncngnanancntnancnancntncncntngnancntngntntngntncngngngnangntncntnanananancntntttt

SEQ ID NO: 142 - RNA, 340 nt, synthetic construct
  source 1..340: mol_type = other RNA; note = Chemically synthesized guide array with guides 3 and 4 for 103-11
  modified_base 1: mod_base = gm
  modified_base 2: mod_base = gm
  modified_base 3: mod_base = um
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
ggtncnanantnancncncntntncntnanananantntnangnanangngngntncntnanananancncntncncntngnangnancntngnanananantntntnantngntncncnantntnanangngntncnanantnancncncntntncntnanananantntnangnanangngngntncntnanananancngntnanangntntntncncncnantnantngntnangncngntncntncncntntncncngngntncnanantnancncncntntncntnanananantntnangnanangngngntncntnanananancntntttt

SEQ ID NO: 143 - RNA, 340 nt, synthetic construct
  source 1..340: mol_type = other RNA; note = Chemically synthesized guide array with guides 3 and 4 for 103-12
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancncncntntnancncngnantntngngntnanangngngntncntngnananancncntncncntngnangnancntngnanananantntntnantngntncncnantntnanangnanancntnancnancncncntntnancncngnantntngngntnanangngngntncntngnananancngntnanangntntntncncncnantnantngntnangncngntncntncncntntncncngnanancntnancnancncncntntnancncngnantntngngntnanangngngntncntngnananancntntttt

SEQ ID NO: 144 - RNA, 340 nt, synthetic construct
  source 1..340: mol_type = other RNA; note = Chemically synthesized guide array with guides 3 and 4 for 103-14
  modified_base 1: mod_base = gm
  modified_base 2: mod_base = um
  modified_base 3: mod_base = um
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gttntntnangnanancncnantngncnanananantncnancnancnancncntncntnanananancncntncncntngnangnancntngnanananantntntnantngntncncnantntnanangntntntntnangnanancncnantngncnanananantncnancnancnancncntncntnanananancngntnanangntntntncncncnantnantngntnangncngntncntncncntntncncngntntntntnangnanancncnantngncnanananantncnancnancnancncntncntnanananancntntttt

SEQ ID NO: 145 - RNA, 340 nt, synthetic construct
  source 1..340: mol_type = other RNA; note = Chemically synthesized guide array with guides 5 and 6 for 103-9
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancncncncntnancngngnanantngngntnangngngngntncntnanananancngntnanantntntncncncnangntnangntnancnananantnananantntntngnangnanancntnancnancncncncntnancngngnanantngngntnangngngngntncntnanananancnangntngntntngntntnancncnananangntntngngncncnantngngngnancnangnanancntnancnancncncncntnancngngnanantngngntnangngngngntncntnanananancntntttt

SEQ ID NO: 146 - RNA, 340 nt, synthetic construct
  source 1..340: mol_type = other RNA; note = Chemically synthesized guide array with guides 5 and 6 for 103-10
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancntncncntngnancntngntntngntncngngngnangntncntnanananancngntnanantntntncncncnangntnangntnancnananantnananantntntngnangnanancntnancnancntncncntngnancntngntntngntncngngngnangntncntnanananancnangntngntntngntntnancncnananangntntngngncncnantngngngnancnangnanancntnancnancntncncntngnancntngntntngntncngngngnangntncntnanananancntntttt

SEQ ID NO: 147 - RNA, 340 nt, synthetic construct
  source 1..340: mol_type = other RNA; note = Chemically synthesized guide array with guides 5 and 6 for 103-11
  modified_base 1: mod_base = gm
  modified_base 2: mod_base = gm
  modified_base 3: mod_base = um
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
ggtncnanantnancncncntntncntnanananantntnangnanangngngntncntnanananancngntnanantntntncncncnangntnangntnancnananantnananantntntngnangngntncnanantnancncncntntncntnanananantntnangnanangngngntncntnanananancnangntngntntngntntnancncnananangntntngngncncnantngngngnancnangngntncnanantnancncncntntncntnanananantntnangnanangngngntncntnanananancntntttt

SEQ ID NO: 148 - RNA, 340 nt, synthetic construct
  source 1..340: mol_type = other RNA; note = Chemically synthesized guide array with guides 5 and 6 for 103-12
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancncncntntnancncngnantntngngntnanangngngntncntngnananancngntnanantntntncncncnangntnangntnancnananantnananantntntngnangnanancntnancnancncncntntnancncngnantntngngntnanangngngntncntngnananancnangntngntntngntntnancncnananangntntngngncncnantngngngnancnangnanancntnancnancncncntntnancncngnantntngngntnanangngngntncntngnananancntntttt

SEQ ID NO: 149 - RNA, 340 nt, synthetic construct
  source 1..340: mol_type = other RNA; note = Chemically synthesized guide array with guides 5 and 6 for 103-14
  modified_base 1: mod_base = gm
  modified_base 2: mod_base = um
  modified_base 3: mod_base = um
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 37^38: mod_base = OTHER; note = phosphorothioate bond
  modified_base 338^339: mod_base = OTHER; note = phosphorothioate bond
  modified_base 339^340: mod_base = OTHER; note = phosphorothioate bond
gttntntnangnanancncnantngncnanananantncnancnancnancncntncntnanananancngntnanantntntncncncnangntnangntnancnananantnananantntntngnangntntntntnangnanancncnantngncnanananantncnancnancnancncntncntnanananancnangntngntntngntntnancncnananangntntngngncncnantngngngnancnangntntntntnangnanancncnantngncnanananantncnancnancnancncntncntnanananancntntttt

SEQ ID NO: 150 - RNA, 340 nt, synthetic construct
  source 1..340: mol_type = other RNA
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancncncncntnancngngnanantngngntnangngngngntncntnanananancncngngnantnancncngnangnangnananancnantntngntnancncncncnantnangnanancntnancnancncncncntnancngngnanantngngntnangngngngntncntnanananancnantnangntncncngntntncntntngncnancnantnancncncncntncnangngncngnanancntnancnancncncncntnancngngnanantngngntnangngngngntncntnanananancntntttt

SEQ ID NO: 151 - RNA, 340 nt, synthetic construct
  source 1..340: mol_type = other RNA; note = Chemically synthesized guide array with guides 7 and 8 for 103-10
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancntncncntngnancntngntntngntncngngngnangntncntnanananancncngngnantnancncngnangnangnananancnantntngntnancncncncnantnangnanancntnancnancntncncntngnancntngntntngntncngngngnangntncntnanananancnantnangntncncngntntncntntngncnancnantnancncncncntncnangngncngnanancntnancnancntncncntngnancntngntntngntncngngngnangntncntnanananancntntttt

SEQ ID NO: 152 - RNA, 340 nt, synthetic construct
  source 1..340: mol_type = other RNA; note = Chemically synthesized guide array with guides 7 and 8 for 103-11
  modified_base 1: mod_base = gm
  modified_base 2: mod_base = gm
  modified_base 3: mod_base = um
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
ggtncnanantnancncncntntncntnanananantntnangnanangngngntncntnanananancncngngnantnancncngnangnangnananancnantntngntnancncncncnantnangngntncnanantnancncncntntncntnanananantntnangnanangngngntncntnanananancnantnangntncncngntntncntntngncnancnantnancncncncntncnangngncngngntncnanantnancncncntntncntnanananantntnangnanangngngntncntnanananancntntttt

SEQ ID NO: 153 - RNA, 340 nt, synthetic construct
  source 1..340: mol_type = other RNA; note = Chemically synthesized guide array with guides 7 and 8 for 103-12
  modified_base 1: mod_base = gm
  modified_base 2: mod_base = OTHER; note = 2'-O-Methyladenosine
  modified_base 3: mod_base = OTHER; note = 2'-O-Methyladenosine
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gaancntnancnancncncntntnancncngnantntngngntnanangngngntncntngnananancncngngnantnancncngnangnangnananancnantntngntnancncncncnantnangnanancntnancnancncncntntnancncngnantntngngntnanangngngntncntngnananancnantnangntncncngntntncntntngncnancnantnancncncncntncnangngncngnanancntnancnancncncntntnancncngnantntngngntnanangngngntncntngnananancntntttt

SEQ ID NO: 154 - RNA, 340 nt, synthetic construct
  source 1..340: mol_type = other RNA; note = Chemically synthesized guide array with guides 7 and 8 for 103-14
  modified_base 1: mod_base = gm
  modified_base 2: mod_base = um
  modified_base 3: mod_base = um
  modified_base 338: mod_base = um
  modified_base 339: mod_base = um
  modified_base 340: mod_base = um
  modified_base 1^2: mod_base = OTHER; note = phosphorothioate bond
  modified_base 2^3: mod_base = OTHER; note = phosphorothioate bond
  modified_base 3^4: mod_base = OTHER; note = phosphorothioate bond
  modified_base 337^338: mod_base = OTHER; note = phosphorothioate bond
  modified_base 338^339: mod_base = OTHER; note = phosphorothioate bond
  modified_base 339^340: mod_base = OTHER; note = phosphorothioate bond
gttntntnangnanancncnantngncnanananantncnancnancnancncntncntnanananancncngngnantnancncngnangnangnananancnantntngntnancncncncnantnangntntntntnangnanancncnantngncnanananantncnancnancnancncntncntnanananancnantnangntncncngntntncntntngncnancnantnancncncncntncnangngncngntntntntnangnanancncnantngncnanananantncnancnancnancncntncntnanananancntntttt

SEQ ID NO: 155 - protein, 7 aa, unidentified
  source 1..7: note = source - SV40
PKKKRKV

SEQ ID NO: 156 - protein, 16 aa, unidentified
  source 1..16: note = source - nucleoplasmin bipartite NLS
KRPAATKKAGQAKKKK

SEQ ID NO: 157 - protein, 9 aa, unidentified
  source 1..9: note = source - c-myc NLS
PAAKRVKLD

SEQ ID NO: 158 - protein, 11 aa, unidentified
  source 1..11: note = source - c-myc NLS
RQRRNELKRSP

SEQ ID NO: 159 - protein, 38 aa, unidentified
  source 1..38: note = source - hRNPA1 M9 NLS
NQSSNFGPMKGGNFGGRSSGPYGGGGQYFAKPRNQGGY

SEQ ID NO: 160 - protein, 42 aa, unidentified
  source 1..42: note = source - Importin-alpha IBB domain
RMRIZFKNKGKDTAELRRRRVEVSVELRKAKKDEQILKRRNV

SEQ ID NO: 161 - protein, 8 aa, unidentified
  source 1..8: note = source - Myoma T protein
VSRKRPRP

SEQ ID NO: 162 - protein, 8 aa, unidentified
  source 1..8: note = source - Myoma T protein
PPKKARED

SEQ ID NO: 163 - protein, 8 aa, unidentified
  source 1..8: note = source - p53
PQPKKKPL

SEQ ID NO: 164 - protein, 12 aa, unidentified
  source 1..12: note = source - mouse c-abl IV
SALIKKKKKMAP

SEQ ID NO: 165 - protein, 5 aa, unidentified
  source 1..5: note = soruce - influenza virus NS1
DRLRR

SEQ ID NO: 166 - protein, 7 aa, unidentified
  source 1..7: note = soruce - influenza virus NS1
PKQKKRK

SEQ ID NO: 167 - protein, 10 aa, unidentified
  source 1..10: note = source - Hepatitis virus delta antigen
RKLKKKIKKL

SEQ ID NO: 168 - protein, 10 aa, unidentified
  source 1..10: note = source - mouse Mx1 protein
REKKKFLKRR

SEQ ID NO: 169 - protein, 20 aa, Homo sapiens
  source 1..20: note = source - human poly(ADP-ribose) polymerase
KRKGDEVDGVDEVAKKKSKK

SEQ ID NO: 170 - protein, 17 aa, Homo sapiens
  source 1..17: note = source - steroid hormone receptor (human) glucocorticoid
RKCLQAGMNLEARKTKK

(19) United States
(12) Patent Application Publication
(10) Pub. No.: US 2024/0352433 A1
(43) Pub. Date: Oct. 24, 2024

(54) ENZYMES WITH HEPN DOMAINS

(71) Applicant: Metagenomi, Inc., Emeryville, CA (US)

(72) Inventors: Brian C. THOMAS, Berkeley, CA (US); Christopher BROWN, Albany, CA (US); Cindy CASTELLE, San Francisco, CA (US); Lisa ALEXANDER, Albany, CA (US); Liliana GONZALEZ-OSORIO, Hercules, CA (US); Morayma TEMOCHE-DIAZ, Oakland, CA (US)

(21) Appl. No.: 18/646,380

(22) Filed: Apr. 25, 2024

Related U.S. Application Data
(63) Continuation of application No. PCT/US2022/078720, filed on Oct. 26, 2022.
(60) Provisional application No. 63/272,500, filed on Oct. 27, 2021.

(51) Int. Cl. C12N 9/22; C12N 15/10; C12N 15/11

(52) U.S. Cl. CPC C12N 9/22; C12N 15/102; C12N 15/11; C12N 2310/20

(57) ABSTRACT

The present disclosure provides for endonuclease enzymes having HEPN domains, as well as methods of using such enzymes or variants thereof.

### CROSS-REFERENCE

This application is a continuation of International Application No. PCT/US2022/078720, entitled “ENZYMES WITH HEPN DOMAINS”, filed on Oct. 26, 2022, which claims the benefit of U.S. Provisional Application No. 63/272,500, entitled “ENZYMES WITH HEPN DOMAINS”, filed on Oct. 27, 2021, which is incorporated by reference herein in its entirety.

### SEQUENCE LISTING

The instant application contains a Sequence Listing which has been submitted electronically in XML format and is hereby incorporated by reference in its entirety. The XML copy, created on Apr. 25, 2024, is named 55921-740_301.xml and is 506,289 bytes in size.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SUMMARY

In some aspects, the present disclosure provides for an engineered nuclease system comprising: (a) an endonuclease comprising an HEPN domain, wherein said endonuclease is derived from an uncultivated microorganism; and (b) an engineered guide ribonucleic acid structure configured to form a complex with said endonuclease comprising: (i) a ribonucleic acid sequence configured to hybridize to a target ribonucleic acid sequence; and (ii) a ribonucleic acid sequence configured to bind to said endonuclease. In some embodiments, said endonuclease comprises a sequence having at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1-15 and 62-84, or a variant thereof. In some embodiments, said endonuclease is not a Cas9 endonuclease, a Cas14 endonuclease, a Cas12a endonuclease, a Cas12b endonuclease, a Cas12c endonuclease, a Cas12d endonuclease, a Cas12e endonuclease, a Cas13a endonuclease, a Cas13b endonuclease, a Cas13c endonuclease, or a Cas13d endonuclease. In some embodiments, said endonuclease has less than 80% identity to a Cas13b endonuclease. In some embodiments, said endonuclease comprises a sequence having at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1, 4, 5, 6, 7, 8, 10, 11, 12, 13, or 15, or a variant thereof. In some embodiments, said engineered guide ribonucleic acid structure comprises a repeat having a least 30, at least 31, at least 32, at least 33, at least 34, at least 35, or at least 36 continuous nucleotides having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 21, 26, 30, 35, 41, 46, 50, 54, 60,122,123, 124, or 125. In some embodiments, said ribonucleic acid sequence configured to hybridize to said target ribonucleic acid sequence comprises at least about 18 to about 26 nucleotides. In some embodiments, said engineered guide ribonucleic acid structure is provided as a sequence comprising: (i) a first copy of said repeat; (ii) said ribonucleic acid sequence configured to hybridize to said target ribonucleic acid sequence; and (iii) a second copy of said repeat. In some embodiments, said engineered guide ribonucleic acid structure comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to non-degenerate nucleotides of any one of SEQ ID NOs: 36, 37, 55, or 61.

In some aspects, the present disclosure provides for an engineered nuclease system comprising, (a) an engineered guide ribonucleic acid structure comprising: (i) a ribonucleic acid sequence configured to hybridize to a target ribonucleic acid sequence; and (ii) a ribonucleic acid sequence configured to bind to an endonuclease; and (b) a class 2, type VI endonuclease configured to bind to said engineered guide ribonucleic acid. In some embodiments, said guide ribonucleic acid sequence is 60-100 nucleotides in length. In some embodiments, aid endonuclease comprises a sequence having at least 75% sequence identity to any one of SEQ ID NOs: 1, 4, 5, 6, 7, 8, 10, 11, 12, or 13, or a variant thereof. In some embodiments, said engineered guide ribonucleic acid structure comprises a repeat having a least 30, at least 31, at least 32, at least 33, at least 34, at least 35, or at least 36 continuous nucleotides having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 21, 26, 30, 35, 41, 46, 50, 54, 60,122,123, 124, or 125. In some embodiments, said ribonucleic acid sequence configured to hybridize to said target ribonucleic acid sequence comprises at least about 18 to about 26 nucleotides. In some embodiments, said engineered guide ribonucleic acid structure is provided as a sequence comprising: (i) a first copy of said repeat; (ii) said ribonucleic acid sequence configured to hybridize to said target ribonucleic acid sequence; and (iii) a second copy of said repeat. In some embodiments, said engineered guide ribonucleic acid structure comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to non-degenerate nucleotides of any one of SEQ ID NOs: 36, 37, 55, or 61. In some embodiments, said endonuclease comprises one or more nuclear localization sequences (NLSs) proximal to an N- or C-terminus of said endonuclease. In some embodiments, said NLS comprises any one of SEQ ID NOs: 155-170. In some embodiments, the system further comprises a single-stranded RNA repair template comprising from 5′ to 3′: a first homology arm comprising a sequence of at least 20 nucleotides 5′ to said target ribonucleic acid sequence, a synthetic RNA sequence of at least 10 nucleotides, and a second homology arm comprising a sequence of at least 20 nucleotides 3′ to said target sequence. In some embodiments, said first or second homology arm comprises a sequence of at least 40, 80,120,150, 200, 300, 500, or 1,000 nucleotides. In some embodiments, said sequence identity is determined by a BLASTP, CLUSTALW, MUSCLE, MAFFT, or CLUSTALW with the parameters of the Smith-Waterman homology search algorithm. In some embodiments, said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment. In some embodiments, the endonuclease is fused at its N- or C-terminus to an additional protein domain. In some embodiments, the additional protein domain is a heterologous domain.

In some aspects, the present disclosure provides for an engineered guide ribonucleic acid polynucleotide comprising: (a) an RNA-targeting segment comprising a nucleotide sequence that is complementary to a target sequence in a target RNA molecule; and (b) a protein-binding segment comprising two complementary stretches of nucleotides that hybridize to form a double-stranded RNA (dsRNA) duplex; wherein said two complementary stretches of nucleotides are covalently linked to one another with intervening nucleotides, and wherein said engineered guide ribonucleic acid polynucleotide is configured to form a complex with an endonuclease comprising sequence having at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1-15 and 62-84, or a variant thereof and target said complex to said target sequence of said target RNA molecule.

In one aspect, the present disclosure provides an engineered nuclease system comprising: (a) an endonuclease comprising an HEPN domain; and (b) an engineered guide ribonucleic acid structure configured to form a complex with the endonuclease comprising: (i) a ribonucleic acid sequence configured to hybridize to a target ribonucleic acid sequence; and (ii) a ribonucleic acid sequence configured to bind to the endonuclease. In some embodiments, the endonuclease comprises a sequence having at least 75% sequence identity to any one of SEQ ID NOs: 1-15 and 62-84. In some embodiments, the endonuclease is derived from an uncultivated microorganism. In some embodiments, the endonuclease is not a Cas9 endonuclease, a Cas14 endonuclease, a Cas12a endonuclease, a Cas12b endonuclease, a Cas12c endonuclease, a Cas12d endonuclease, a Cas12e endonuclease, a Cas13a endonuclease, a Cas13b endonuclease, a Cas13c endonuclease, or a Cas13d endonuclease. In some embodiments, the endonuclease has less than 80% identity to a Cas13b endonuclease.

In another aspect, the present disclosure provides an engineered nuclease system comprising, (a) an engineered guide ribonucleic acid structure comprising: (i) a ribonucleic acid sequence configured to hybridize to a target ribonucleic acid sequence; and (ii) a ribonucleic acid sequence configured to bind to an endonuclease; and (b) a class 2, type VI endonuclease configured to bind to the engineered guide ribonucleic acid. In some embodiments, the guide ribonucleic acid sequence is 60-100 nucleotides in length. In some embodiments, the endonuclease comprises one or more nuclear localization sequences (NLSs) proximal to an N- or C-terminus of the endonuclease. In some embodiments, the NLS comprises a sequence selected from SEQ ID NOs: 155-170. In some embodiments, the engineered nuclease system further comprises a single-stranded RNA repair template comprising from 5′ to 3′: a first homology arm comprising a sequence of at least 20 nucleotides 5′ to the target ribonucleic acid sequence, a synthetic RNA sequence of at least 10 nucleotides, and a second homology arm comprising a sequence of at least 20 nucleotides 3′ to the target sequence. In some embodiments, the first or second homology arm comprises a sequence of at least 40, 80,120,150, 200, 300, 500, or 1,000 nucleotides. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment. In some embodiments, the endonuclease is fused at its N- or C-terminus to an additional protein domain. In some embodiments, the additional protein domain is a heterologous domain. In another aspect, the present disclosure provides an engineered guide ribonucleic acid polynucleotide comprising: (a) an RNA-targeting segment comprising a nucleotide sequence that is complementary to a target sequence in a target RNA molecule; and (b) a protein-binding segment comprising two complementary stretches of nucleotides that hybridize to form a double-stranded RNA (dsRNA) duplex; wherein the two complementary stretches of nucleotides are covalently linked to one another with intervening nucleotides, and wherein the engineered guide ribonucleic acid polynucleotide is configured to form a complex with an endonuclease comprising sequence having at least 75% sequence identity to any one of SEQ ID NOs: 1-15 and 62-84 and target the complex to the target sequence of the target RNA molecule. In some embodiments, the RNA-targeting segment is positioned 5′ of both of the two complementary stretches of nucleotides. In another aspect, the present disclosure provides a deoxyribonucleic acid polynucleotide encoding an engineered guide ribonucleic acid polynucleotide or structure described herein. In another aspect, the present disclosure provides a nucleic acid comprising an engineered nucleic acid sequence optimized for expression in an organism, wherein the nucleic acid encodes an endonuclease comprising a sequence having at least 75% sequence identity to any one of SEQ ID NOs: 1-15 and 62-84. In some embodiments, the endonuclease comprises a sequence encoding one or more nuclear localization sequences (NLSs) proximal to an N- or C-terminus of the endonuclease. In some embodiments, the NLS comprises a sequence selected from SEQ ID NOs: 155-170. In some embodiments, the organism is prokaryotic, bacterial, eukaryotic, fungal, plant, mammalian, rodent, or human. In another aspect, the present disclosure provides a vector comprising a nucleic acid described herein. In some embodiments, the vector further comprises a nucleic acid encoding an engineered guide ribonucleic acid structure configured to form a complex with the endonuclease comprising: (a) a ribonucleic acid sequence configured to hybridize to a target ribonucleic acid sequence; and (b) a ribonucleic acid sequence configured to bind to the endonuclease. In some embodiments, the vector is a plasmid, a minicircle, a CELiD, an adeno-associated virus (AAV) derived virion, or a lentivirus. In another aspect, the present disclosure provides a cell comprising a vector described herein. In another aspect, the present disclosure provides a method of manufacturing an endonuclease, comprising cultivating a cell described herein. In another aspect, the present disclosure provides a method for binding, cleaving, marking, or modifying a single-stranded ribonucleic acid polynucleotide, comprising: contacting the single-stranded ribonucleic acid polynucleotide with a class 2, type VI endonuclease in complex with an engineered guide ribonucleic acid structure configured to bind to the endonuclease and the single-stranded ribonucleic acid polynucleotide. In some embodiments, the single-stranded ribonucleic acid polynucleotide comprises a protospacer flanking site (PFS). In some embodiments, the single-stranded ribonucleic acid polynucleotide comprises a sequence complementary to a sequence of the engineered guide ribonucleic acid structure and a PFS. In some embodiments, the PFS is directly adjacent to the sequence complementary to the sequence of the engineered guide ribonucleic acid structure. In some embodiments, the single-stranded ribonucleic acid polynucleotide does not comprise a protospacer flanking site (PFS). In some embodiments, the class 2, type VI endonuclease is not a Cas9 endonuclease, a Cas14 endonuclease, a Cas12a endonuclease, a Cas12b endonuclease, a Cas12c endonuclease, a Cas12d endonuclease, a Cas12e endonuclease, a Cas13a endonuclease, a Cas13b endonuclease, a Cas13c endonuclease, or a Cas13d endonuclease. In some embodiments, the single-stranded ribonucleic acid polynucleotide is a eukaryotic, plant, fungal, mammalian, rodent, or human single-stranded ribonucleic acid polynucleotide. In another aspect, the present disclosure provides a method of modifying a target nucleic acid locus, the method comprising delivering to the target nucleic acid locus an engineered nuclease system described herein, wherein the endonuclease is configured to form a complex with the engineered guide ribonucleic acid structure, and wherein the complex is configured such that upon binding of the complex to the target nucleic acid locus, the complex modifies the target nucleic locus. In some embodiments, modifying the target nucleic acid locus comprises binding, nicking, cleaving, or marking the target nucleic acid locus. In some embodiments, the target nucleic acid locus comprises deoxyribonucleic acid (DNA) or ribonucleic acid (RNA). In some embodiments, the target nucleic acid comprises genomic DNA, genomic RNA, viral DNA, viral RNA, bacterial DNA, or bacterial RNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, delivering the engineered nuclease system to the target nucleic acid locus comprises delivering a nucleic acid described herein or a vector described herein. In some embodiments, delivering the engineered nuclease system to the target nucleic acid locus comprises delivering a nucleic acid comprising an open reading frame encoding the endonuclease. In some embodiments, the nucleic acid comprises a promoter to which the open reading frame encoding the endonuclease is operably linked. In some embodiments, delivering the engineered nuclease system to the target nucleic acid locus comprises delivering a capped mRNA containing the open reading frame encoding the endonuclease. In some embodiments, delivering the engineered nuclease system to the target nucleic acid locus comprises delivering a translated polypeptide. In some embodiments, delivering the engineered nuclease system to the target nucleic acid locus comprises delivering a deoxyribonucleic acid (DNA) encoding the engineered guide ribonucleic acid structure operably linked to a ribonucleic acid (RNA) pol III promoter. In some embodiments, the endonuclease induces a single-stranded break at or proximal to the target locus. In another aspect, the present disclosure provides an engineered guide ribonucleic acid polynucleotide comprising: (a) an RNA-targeting segment comprising a nucleotide sequence that is complementary to a target sequence in a target RNA molecule; and (b) a protein-binding segment comprising two complementary stretches of nucleotides that hybridize to form a double-stranded RNA (dsRNA) duplex, wherein the two complementary stretches of nucleotides are covalently linked to one another with intervening nucleotides, and wherein the engineered guide ribonucleic acid polynucleotide is configured to form a complex with a class 2, type VI endonuclease and target the complex to the target sequence of the target RNA molecule.

In another aspect, the present disclosure provides a system for generating an edited immune cell, comprising: (a) an RNA-guided endonuclease; (b) an engineered guide ribonucleic acid polynucleotide described herein configured to bind the RNA-guided endonuclease; and (c) a single-stranded RNA repair template comprising first and second homology arms flanking a sequence encoding a chimeric antigen receptor (CAR). In some embodiments, the cell is a peripheral blood mononuclear cell, a T-cell, an NK cell, a hematopoietic stem cell (HSCT), or a B-cell. In some embodiments, the RNA-guided endonuclease is a class II, type VI endonuclease.

In some embodiments, the RNA-guided endonuclease comprises an HEPN domain.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A-1C depicts the MG103 Family. (FIG. 1A) depicts a multiple alignment of MG103 effectors representatives showing domains compositions and conservation of the HEPN catalytic residues critical for function for a single stranded RNA cleavage. (FIG. 1B) depicts a representation of a CRISPR-containing contig with genomic context surrounding the CRISPR array and the effector (example of MG103-2). (FIG. 1C) depicts folding of the Direct repeat of MG103-2.

FIG. 2A-2C depicts the MG105 Family. (FIG. 2A) depicts a multiple alignment of MG105 effector representative showing conservation of the HEPN catalytic residues critical for function for a single stranded RNA cleavage activity. (FIG. 2B) depicts a representation of a CRISPR-containing contig with genomic context surrounding the CRISPR array and the effector (example of MG105-1). (FIG. 2C) depicts folding of the Direct repeat of MG105-1.

FIG. 3 depicts a phylogenetic tree inferred from a multiple sequence alignment of Cas13d protein sequences. Reference Cas13d sequences were included in the tree for classification purposes. Closed dark circles indicate novel candidates.

FIG. 4 depicts a fluorescence-based mRNA cis-cleavage assay. Minimal arrays targeting deGFP mRNA and nucleases were transcribed and translated in vitro with PURExpress (NEB). Mature crRNAs were processed by the translated nuclease. After a 20 min incubation at 37° C., the deGFP mRNA was added to each reaction to form an activated complex with the mature targeting crRNA. Fluorescence signal of translated deGFP mRNA was measured at 37° C. for 3 hours in 3-minute intervals. An active complex (+crRNA) is expected to exhibit a robust decrease in fluorescence compared to the apo conditions (−crRNA). Immediately after the 3-hour incubation, all reactions were stored at −80° C. until ready for RNA extractions. RNA extractions were treated with T4 PNK to mono-phosphorylate the 5′ end of the mature crRNA and sequenced to determine the active crRNA processing.

FIG. 5 depicts in vitro deGFP mRNA cleavage. Fluorescence was measured at 485/20 excitation and 528/20 emission every 3-5 min for 2-3 hours. MA2X1 refers to the minimal array designs that have two repeats and one targeting spacer. Repeats were tested in the forward (FWD) and reverse (REV) orientations. Apo and non-targeting (NT) conditions generated high fluorescence while targeting active conditions exhibited a robust decrease in fluorescence. The data was subtracted from background fluorescence (Non template conditions) and each curve was fit to a plateau followed by one phase exponential decay. One replicate of each condition was tested.

FIG. 6 depicts deGFP fluorescence knock down by targeted cleavage. MA2X1 refers to the minimal array designs that have two repeats and one targeting spacer. Repeats were tested in the forward (FWD) and reverse (REV) orientations. Fluorescence decrease percentages were quantified from the plateau parameter. The Apo plateau value was subtracted from each condition then divided by the apo plateau and multiplied by 100. The percentages from the targeting and non-targeting (NT) reactions were plotted in solid and striped bars, respectively. Targeted cleavage resulted in up to 97.70% decrease in fluorescence. One replicate of each condition was tested.

FIG. 7 depicts a fluorescence-based mRNA trans-cleavage assay. Minimal arrays targeting the 101 nt activator RNA and nucleases were transcribed and translated in vitro with PURExpress (NEB). Mature crRNAs were processed by the translated nucleases. After a 20 min incubation at 37° C., the deGFP mRNA and activator RNA were added to each reaction to form an activated complex with the mature targeting crRNA. deGFP mRNA was not targeted by the minimal array, it was present as a bystander RNA that can be cleaved by trans activity. The fluorescence signal of translated deGFP mRNA was measured at 37° C. for 3 hours in 3-minute intervals. An active complex (+crRNA) is expected to exhibit a robust decrease in fluorescence compared to the apo conditions (−crRNA).

FIG. 8 depicts in vitro deGFP mRNA cis vs. trans-cleavage. Apo reactions were plotted in circles. Reactions plotted in squares tested cleavage with minimal arrays targeting the deGFP mRNA. Reactions plotted in triangles tested cleavage with minimal arrays not targeting the deGFP mRNA. Reactions plotted in diamonds tested trans-cleavage of deGFP mRNA with activated nuclease complexes, spacers in the minimal arrays are not complementary to deGFP mRNA. Apo and non-targeting conditions exhibited high fluorescence compared to cis and trans cleavage reactions. The data was subtracted from background fluorescence (Non template conditions) and each curve except for MG105-1 reactions were fit to a plateau followed by one phase exponential decay.

FIG. 9 depicts deGFP fluorescence knock down by cis vs. trans-cleavage. Fluorescence decrease percentages were quantified from the plateau parameter. The Apo plateau value was subtracted from each condition then divided by the apo plateau and multiplied by 100. For MG105-1, not enough data points were collected for a proper fit of the data to a plateau followed by one phase exponential decay. Instead, the Apo max fluorescence signal was subtracted from each condition then divided by the apo max fluorescence signal and multiplied by 100. Cis and Trans-cleavage results showed comparable decrease in fluorescence. One replicate of each condition was tested.

FIG. 10A-10C depicts RNAseq Analysis. Reads were mapped to minimal array sequences used in each reaction. The crRNA processing boundaries were denoted by white double pointed arrows. FIGS. 10A and 10B demonstrate that MG103 nucleases process crRNA on the 5′ end of the repeat and the 3′ end of the spacer. The resulting active spacer lengths were 21 or 26 nucleotides and the active repeat length was 30 nucleotides. FIG. 10C demonstrates that MG105-1 processes crRNA differently. The crRNA is trimmed 10 nucleotides on 5′ of the spacer leaving behind an untrimmed repeat sequence.

FIG. 11 depicts an overview of the protocol for testing Type VI nucleases in HEK293 T cells.

FIG. 12A-12B depicts GFP knockdown in HEK293T cells using a cas13 positive control. The suitability of the assay was validated by using guided and unguided positive controls. FIG. 12A depicts an overlapping distribution of GFP fluorescence for the guided (plasmid guide, chemically synthesized guide) and unguided conditions (Apo) showing a shift to lower fluorescence for the guided conditions. FIG. 12B depicts quantification of FIG. 12A, showing the means of each population. “Plasmid guide” and “plasmid” refer to an array encoded in a plasmid. “Chem. synt. guide” and “chem. synthesized” refer to an array chemically synthesized with 5′ and 3′ modifications.

FIG. 13A-13J depicts GFP knockdown in HEK293T cells with the positive control and MG nucleases. FIGS. 13A through 13E: The overlapping distributions of GFP fluorescence for the guided (arrays 1-4 and arrays 5-8) and unguided conditions (Apo) show a shift to lower fluorescence for the guided conditions. FIGS. 13A through 13E represent each candidate. FIGS. 13F through 13J: The overlapping distributions of GFP fluorescence for the guided (array 1-2, 3-4, 5-6, or 7-8) and unguided conditions (Apo) show a shift to lower fluorescence for the guided conditions. FIGS. 13F through 13J represent each candidate.

FIG. 14A-14K depicts quantification of GFP knockdown in HEK293T cells with the positive control and MG nucleases. FIGS. 14A through 14E: Quantification and distribution of GFP fluorescence for the guided (arrays 1-4 and arrays 5-8) and unguided conditions (Apo) show lower median values for guided conditions. The differences of Apo vs. guided conditions were significantly different for all conditions. Numbers shown represent the median fluorescence of each population. FIGS. 14A through 14E represent each candidate. FIGS. 14F through 14K: Quantification and distribution of GFP fluorescence for the highest knockdown chemically synthesized guide array (either 1-2, 3-4, 5-6, or 7-8) and unguided conditions (Apo). 103-9, 103-11, 103-12, and 103-14 show lower median values for guided conditions than Apo control. The differences of Apo vs. guided conditions were significantly different for all conditions except for 103-10, where the guided arrays all had the same or higher fluorescence than Apo. The lines and associated values shown represent the median fluorescence of each population of 25,000 cells. FIG. 14F represents positive control and FIGS. 14G through 14K represent each candidate.

FIG. 15A depicts GFP knockdown in HEK293T cells with the positive control and MG nucleases. The knockdown efficiency was calculated setting the median for the Apo condition as 100% GFP expression. 103-3 shows a similar level of repression as the positive control. 103-3 repression is followed by 103-6, 103-7, and 103-2. FIG. 15B depicts GFP knockdown in HEK293T cells with the positive control and MG novel nucleases using chemically synthesized guides. The knockdown efficiency was calculated setting the median for the Apo condition as 100% GFP expression. 103-12 shows similar knockdown to the positive control.

### BRIEF DESCRIPTION OF THE SEQUENCE LISTING

The Sequence Listing filed herewith provides exemplary polynucleotide and polypeptide sequences for use in methods, compositions and systems according to the disclosure. Below are exemplary descriptions of sequences therein. MG105

SEQ ID NOs: 1-2 show the full-length peptide sequences of MG105 nucleases.

SEQ ID NOs: 56-61 show the nucleotide sequences of DNA templates used for the in vitro transcription and translation of MG105 nucleases described herein. MG103

SEQ ID NOs: 3-15 and 62-84 show the full-length peptide sequences of MG103 nucleases.

SEQ ID NOs: 18-55 show the nucleotide sequences of DNA templates used for the in vitro transcription and translation of MG103 nucleases described herein.

SEQ ID NOs: 86-89 and 135-154 show the nucleotide sequences of chemically synthesized RNA guides suitable for use with MG103 nucleases described herein.

SEQ ID NOs: 90-105 show the nucleotide sequences of CRISPR arrays targeting eGFP suitable for use with MG103 nucleases described herein.

SEQ ID NOs: 106-113 show the nucleotide sequences of plasmids encoding CRISPR arrays targeting eGFP suitable for use with MG103 nucleases described herein.

SEQ ID NOs: 122-125 show the repeat sequences identified by the MG103 nucleases described herein.

SEQ ID NOs: 126-134 show codon-optimized DNA sequences encoding MG103 nucleases described herein. MG106

SEQ ID NOs: 171-172 show the full-length peptide sequences of MG106 nucleases.

SEQ ID NOs: 173-180 show the nucleotide sequences of DNA templates used for the in vitro transcription and translation of MG106 nucleases described herein.

### Assay Materials

SEQ ID NOs: 16-17 show the nucleotide sequences of RNA templates used to assess the cleavage activity of nuclease systems described herein.

SEQ ID NO: 85 shows the full-length peptide sequence of a GFP-PEST reporter protein useful to assess the RNA cleavage activity in mammalian cells of nuclease systems described herein.

SEQ ID NOs: 114-121 shows the nucleotide sequences of ueGFP-targeting spacer sequences useful to assess the RNA cleavage activity in mammalian cells of nuclease systems described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4^{th} Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6^{th} Edition (R.I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof See, e.g., Sambrook et al., 1989, Molecular Cloning: A Laboratory Manual, 18.1-18.88.

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. Eukaryotic basal promoters can contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

The term “tracrRNA” or “tracr sequence”, as used herein, can generally refer to a nucleic acid with at least about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes S. aureus, etc or SEQ ID NOs: 5476-5511). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. The strand of a single-stranded target polynucleotide that is complementary to and hybridizes with the guide nucleic acid may be called the complementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation I of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation(E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

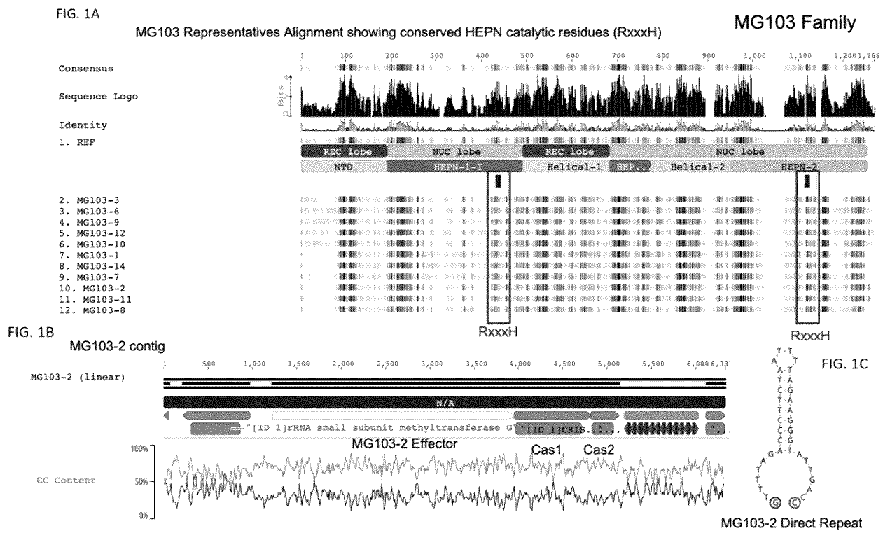

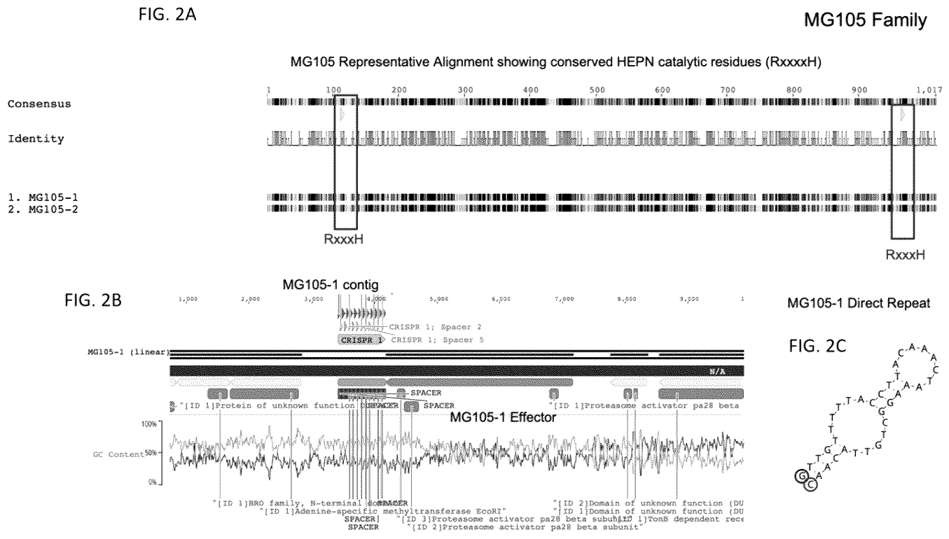

Included in the current disclosure are variants of any of the enzyme described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g. non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to any one of the endonuclease protein sequences described herein (e.g. MG103 or MG105 family endonucleases described herein). In some embodiments, such conservatively substituted variants are functional variants. Such functional variants can encompass sequences with substitutions such that the activity of critical active site residues of the endonuclease are not disrupted. In some embodiments, a functional variant of any of the proteins described herein lacks substitution of at least one of the conserved or functional residues called out in FIG. 1 or 2. In some embodiments, a functional variant of any of the proteins described herein lacks substitution of all of the conserved or functional residues called out in in FIG. 1 or 2.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

As used herein, the term “HEPN domain” generally refers to an endonuclease domain having characteristic histidine and arginine residues. An HEPN domain can generally be identified by alignment to documented domain sequences, structural alignment to proteins with annotated domains, or by comparison to Hidden Markov Models (HMMs) built based on known domain sequences (e.g., Pfam HMM PF05168 for domain HEPN)

As used herein, the term “protospacer flanking site (PFS)” generally refers to a sequence motif adjacent to a target RNA protospacer that affects nuclease activity. The PFS is typically found at one end of the RNA protospacer. A nuclease described herein may or may not have a sequence preference at the PFS position. In some instances, the PFS positively affects nuclease activity. In some cases, any of the nucleic acid sequences targeted herein can comprise a PFS sequence adjacent to a target nucleic acid site. In some cases, any of the nucleic acid sequences targeted herein can comprise a PFS sequence 3′ to a target nucleic acid site. In some instances, the PFS negatively affects nuclease activity. In some cases, any of the nucleic acid sequences targeted herein can lack a PFS sequence adjacent to a target nucleic acid site. In some cases, any of the nucleic acid sequences targeted herein can lack a PFS sequence 3′ to a target nucleic acid site.

Included in the current disclosure are hybrid, chimeric, or fusion protein variants comprising any of the endonucleases described herein. Such hybrid, chimeric, or fusion protein variants can comprise: (i) any of the endonucleases described herein; (ii) an additional protein domain fused to the N- or C-terminus of the endonuclease; and (iii) an optional linker domain between the endonuclease and the additional protein domain. In some cases, the additional protein domain is a domain heterologous to the endonuclease. Additional protein domains contained in hybrid, chimeric, or fusion protein variants according to the disclosure can include ligase domains, repair protein domains, methyltransferase domains, recombinase domains, transposase domains, argonaute domains, cytidine deaminase domains, adenine deaminase domains, double-stranded RNA-specific adenosine deaminase (ADAR) domains, a retron, a group II intron, phosphatase domains, phosphorylase domains, sulfurylase domains, kinase domains, polymerase domains, exonuclease domains, helicase domains, demethylase domains, translation co-activator domains, RNA polymerase domains, reporter protein domains, fluorescent protein domains, ligand binding protein domains, signal peptide domains, subcellular localization sequences, or antibody epitopes.

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity. In some cases efficient nuclease targeting of a particular target nucleic acid sequence can require (i) complementary hybridization between the first 6-8 nucleic acids of the target (the target seed) and the crRNA guide; and (ii) the presence of a protospacer flanking site within a defined vicinity of the target seed. In some cases efficient nuclease targeting of a particular target nucleic acid sequence can require (i) complementary hybridization between the first 6-8 nucleic acids of the target (the target seed) and the crRNA guide; and (ii) the absence of a protospacer flanking site within a defined vicinity of the target seed.

Class I CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that comprises a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRIPSR-Cas systems have RNA-guided RNA endonucleases. Instead of RuvC-like domains, the single polypeptide effector of Type VI systems (e.g. Cas13) comprises two HEPN ribonuclease domains. Differing from both Type II and V systems, Type VI systems may not require a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA. Type VI CRISPR-Cas systems may or may not additionally have a protospacer flanking site (PFS) requirement that affects nuclease activity.

### MG Enzymes

Type VI CRISPR systems are quickly being adopted for use in a variety of genome editing applications. These programmable nucleases are part of adaptive microbial immune systems, the natural diversity of which has been largely unexplored. Novel families of Type VI CRISPR enzymes were identified through a large-scale analysis of metagenomes collected from a variety of complex environments, and representatives of these were developed systems into gene-editing platforms. The majority of these systems come from uncultivated organisms, some of which encode a divergent Type VI effector within the same CRISPR operon.

In some aspects, the present disclosure provides for novel Type VI candidates. These candidates may represent one or more novel subtypes and some sub-families may have been identified. These nucleases are less than about 1,000 amino acids in length. These novel subtypes may be found in the same CRISPR locus as documented Type VI effectors. HEPN catalytic residues may have been identified for the novel Type VI candidates, and these novel Type VI candidates may not require tracrRNA.

In some aspects, the present disclosure provides for smaller Type VI effectors. Such effectors may be small putative effectors. These effectors may simplify delivery and may extend therapeutic applications.

In some aspects, the present disclosure provides for a novel type VI effector. Such an effector may be MG103 as described herein (see FIG. 1). Such an effector may be MG105 as described herein (see FIG. 2).

In one aspect, the present disclosure provides for an engineered nuclease system discovered through metagenomic sequencing. In some cases, the metagenomic sequencing is conducted on samples. In some cases, the samples may be collected by a variety of environments. Such environments may be a human microbiome, an animal microbiome, environments with high temperatures, environments with low temperatures. Such environments may include sediment.

### MG103 Enzymes

In one aspect, the present disclosure provides for an engineered nuclease system comprising an endonuclease. In some cases, the endonuclease is a Type II, Class VI endonuclease. The endonuclease may comprise a first HEPN domain. The endonuclease may comprise a second HEPN domain. The endonuclease may comprise a first HEPN domain and a second HEPN domain.

In some cases, the endonuclease may comprise a variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 3-15 and 62-84. In some cases, the endonuclease may be substantially identical to any one of SEQ ID NOs: 3-15 and 62-84. In some cases, the endonuclease may comprise a peptide motif substantially identical to any one of SEQ ID NOs: 3-15 and 62-84.

In some cases, the endonuclease may comprise a variant having one or more nuclear localization sequences (NLSs). The NLS may be proximal to the N- or C-terminus of said endonuclease. The NLS may be appended N-terminal or C-terminal to any one of SEQ ID NOs: 3-15 and 62-84, or to a variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 3-15 and 62-84. The NLS may be an SV40 large T antigen NLS. The NLS may be a c-myc NLS. The NLS can comprise a sequence with at least about 80%, at least about 85%, at least about 90%, at least about 95%, at least about 99% identity to any one of SEQ ID NOs: 155-170. The NLS can comprise a sequence substantially identical to any one of SEQ ID NOs: 155-170. The NLS can comprise any of the sequences in Table 1 below, or a combination thereof:

TABLE 1
Example NLS Sequences that can be used with
Effectors According to the Disclosure

| | NLS amino | SEQ ID |
|---|---|---|
| Source | acid sequence | NO: |
| SV40 | PKKKRKV | 155 |
| nucleoplasmin | KRPAATKKAGQAKKKK | 156 |
| bipartite NLS | | |
| c-myc NLS | PAAKRVKLD | 157 |
| c-myc NLS | RQRRNELKRSP | 158 |
| hRNPA1 M9 NLS | NQSSNFGPMKGGNFGG | 159 |
| | RSSGPYGGGGQYFAKP | |
| | RNQGGY | |
| Importin-alpha | RMRIZFKNKGKDTAEL | 160 |
| IBB domain | RRRRVEVSVELRKAKK | |
| | DEQILKRRNV | |
| Myoma T protein | VSRKRPRP | 161 |
| Myoma T protein | PPKKARED | 162 |
| p53 | PQPKKKPL | 163 |
| mouse c-abl IV | SALIKKKKKMAP | 164 |
| influenza virus | DRLRR | 165 |
| NS1 | | |
| influenza virus | PKQKKRK | 166 |
| NS1 | | |
| Hepatitis virus | RKLKKKIKKL | 167 |
| delta antigen | | |
| mouse Mx1 | REKKKFLKRR | 168 |
| protein | | |
| human poly | KRKGDEVDGVDEVAKK | 169 |
| (ADP-ribose) | KSKK | |
| polymerase | | |
| steroid hormone | RKCLQAGMNLEARKTK | 170 |
| receptor (human) | K | |
| glucocorticoid | | |

In some cases, sequence identity may be determined by the BLASTP, CLUSTALW, MUSCLE, MAFFT, Novafold, or CLUSTALW with the parameters of the Smith-Waterman homology search algorithm. The sequence identity may be determined by the BLASTP algorithm using parameters of a wordlength (W) of 3, an expectation (E) of 10, and using a BLOSUM62 scoring matrix setting gap costs at existence of 11, extension of 1, and using a conditional compositional score matrix adjustment.

In some cases, the system above may comprise at least one engineered synthetic guide ribonucleic acid (sgRNA) capable of forming a complex with the endonuclease bearing a targeting region complementary to a cleavage sequence. In some cases, the targeting region is located at the 5′ end of the sgRNA. In some cases, the targeting region is located at the 3′ end of the sgRNA. In some cases, the cleavage sequence may comprise a protospacer flanking site (PFS) sequence compatible with the endonuclease. In some cases, the cleavage sequence may not comprise a protospacer flanking site (PFS) sequence compatible with the endonuclease. In some cases, the targeting region may be 18-30 nucleotides in length. The sgRNA may comprise a crRNA repeat region adjacent to the targeting region and capable of binding the endonuclease. The sgRNA may comprise a non-natural guide nucleic acid sequence capable of hybridizing to a target sequence in a cell.

In some cases, the system above may comprise two different sgRNAs targeting a first region and a second region for cleavage in a target RNA locus, wherein the second region is 3′ to the first region. In some cases, the system above may comprise a single-stranded RNA repair template comprising from 5′ to 3′: a first homology arm comprising a sequence of at least about 20 (e.g., at least about 40, 80,120,150, 200, 300, 500, or 1kb) nucleotides 5′ to the first region, a synthetic RNA sequence of at least about 10 nucleotides, and a second homology arm comprising a sequence of at least about 20 (e.g., at least about 40, 80,120,150, 200, 300, 500, or 1kb) nucleotides 3′ to the second region.

In another aspect, the present disclosure provides a method for modifying a target nucleic acid locus. The method may comprise delivering to the target nucleic acid locus any of the non-natural systems disclosed herein, including an enzyme and at least one synthetic guide RNA (sgRNA) disclosed herein. The enzyme may form a complex with the at least one sgRNA, and upon binding of the complex to the target nucleic acid locus, may modify the target nucleic acid locus. Delivering the enzyme to said locus may comprise transfecting a cell with the system or nucleic acids encoding the system. Delivering the nuclease to said locus may comprise electroporating a cell with the system or nucleic acids encoding the system. Delivering the nuclease to said locus may comprise incubating the system in a buffer with a nucleic acid comprising the locus of interest. In some cases, the target nucleic acid locus comprises deoxyribonucleic acid (DNA) or ribonucleic acid (RNA). The target nucleic acid locus may comprise genomic DNA, genomic RNA, viral DNA, viral RNA, bacterial DNA, or bacterial RNA. The target nucleic acid locus may be within a cell. The target nucleic acid locus may be in vitro. The target nucleic acid locus may be within a eukaryotic cell or a prokaryotic cell. The cell may be an animal cell, a human cell, bacterial cell, archaeal cell, or a plant cell. The enzyme may induce a single or double-stranded break at or proximal to the target locus of interest.

In cases where the target nucleic acid locus may be within a cell, the enzyme may be supplied as a nucleic acid containing an open reading frame encoding the enzyme having a HEPN domain having at least about 75% (e.g., at least about 90%, at least about 91%, at least about 92%, at least about 93%, at least about 94%, at least about 95%, at least about 96%, at least about 97%, at least about 98%, at least about 99%) identity to any one of SEQ ID NOs: 3-15 and 62-84. The deoxyribonucleic acid (DNA) containing an open reading frame encoding said endonuclease may comprise a sequence substantially identical to any of SEQ ID NOs: 3-15 and 62-84 or at variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 3-15 and 62-84. In some cases, the nucleic acid comprises a promoter to which the open reading frame encoding the endonuclease is operably linked. The promoter may be a CMV, EF1a, SV40, PGK1, Ubc, human beta actin, CAG, TRE, or CaMKIIa promoter. The endonuclease may be supplied as a capped mRNA containing said open reading frame encoding said endonuclease. The endonuclease may be supplied as a translated polypeptide. The at least one engineered sgRNA may be supplied as deoxyribonucleic acid (DNA) containing a gene sequence encoding said at least one engineered sgRNA operably linked to a ribonucleic acid (RNA) pol III promoter. In some cases, the organism may be eukaryotic. In some cases, the organism may be fungal. In some cases, the organism may be human.

In some cases, the present disclosure may provide for an expression cassette comprising the system disclosed herein, or the nucleic acid described herein. In some cases, the expression cassette or nucleic acid may be supplied as a vector. In some cases, the expression cassette, nucleic acid, or vector may be supplied in a cell.

### MG105 Enzymes

In one aspect, the present disclosure provides for an engineered nuclease system comprising an endonuclease. In some cases, the endonuclease is a Type II, Class VI endonuclease. The endonuclease may comprise a first HEPN domain. The endonuclease may comprise a second HEPN domain. The endonuclease may comprise a first HEPN domain and a second HEPN domain.

In some cases, the endonuclease may comprise a variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1-2. In some cases, the endonuclease may be substantially identical to any one of SEQ ID NOs: 1-2. In some cases, the endonuclease may comprise a peptide motif substantially identical to any one of SEQ ID NOs: 1-2.

In some cases, the endonuclease may comprise a variant having one or more nuclear localization sequences (NLSs). The NLS may be proximal to the N- or C-terminus of said endonuclease. The NLS may be appended N-terminal or C-terminal to any one of SEQ ID NOs: 1-2, or to a variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1-2. The NLS may be an SV40 large T antigen NLS. The NLS may be a c-myc NLS. The NLS can comprise a sequence with at least about 80%, at least about 85%, at least about 90%, at least about 95%, at least about 99% identity to any one of SEQ ID NOs: 155-170. The NLS can comprise a sequence substantially identical to any one of SEQ ID NOs: 155-170. The NLS can comprise any of the sequences in Table 1, or a combination thereof.

In some cases, sequence identity may be determined by the BLASTP, CLUSTALW, MUSCLE, MAFFT, Novafold, or CLUSTALW with the parameters of the Smith-Waterman homology search algorithm. The sequence identity may be determined by the BLASTP algorithm using parameters of a wordlength (W) of 3, an expectation (E) of 10, and using a BLOSUM62 scoring matrix setting gap costs at existence of 11, extension of 1, and using a conditional compositional score matrix adjustment.

In some cases, the system above may comprise at least one engineered synthetic guide ribonucleic acid (sgRNA) capable of forming a complex with the endonuclease bearing a targeting region complementary to a cleavage sequence. In some cases, the targeting region is located at the 5′ end of the sgRNA. In some cases, the targeting region is located at the 3′ end of the sgRNA. In some cases, the cleavage sequence may comprise a protospacer flanking site (PFS) sequence compatible with the endonuclease. In some cases, the cleavage sequence may not comprise a protospacer flanking site (PFS) sequence compatible with the endonuclease. In some cases, the targeting region may be 18-30 nucleotides in length. The sgRNA may comprise a crRNA repeat region adjacent to the targeting region and capable of binding the endonuclease. The sgRNA may comprise a non-natural guide nucleic acid sequence capable of hybridizing to a target sequence in a cell.

In some cases, the system above may comprise two different sgRNAs targeting a first region and a second region for cleavage in a target RNA locus, wherein the second region is 3′ to the first region. In some cases, the system above may comprise a single-stranded RNA repair template comprising from 5′ to 3′: a first homology arm comprising a sequence of at least about 20 (e.g., at least about 40, 80,120,150, 200, 300, 500, or 1kb) nucleotides 5′ to the first region, a synthetic RNA sequence of at least about 10 nucleotides, and a second homology arm comprising a sequence of at least about 20 (e.g., at least about 40, 80,120,150, 200, 300, 500, or 1kb) nucleotides 3′ to the second region.

In another aspect, the present disclosure provides a method for modifying a target nucleic acid locus. The method may comprise delivering to the target nucleic acid locus any of the non-natural systems disclosed herein, including an enzyme and at least one synthetic guide RNA (sgRNA) disclosed herein. The enzyme may form a complex with the at least one sgRNA, and upon binding of the complex to the target nucleic acid locus, may modify the target nucleic acid locus. Delivering the enzyme to said locus may comprise transfecting a cell with the system or nucleic acids encoding the system. Delivering the nuclease to said locus may comprise electroporating a cell with the system or nucleic acids encoding the system. Delivering the nuclease to said locus may comprise incubating the system in a buffer with a nucleic acid comprising the locus of interest. In some cases, the target nucleic acid locus comprises deoxyribonucleic acid (DNA) or ribonucleic acid (RNA). The target nucleic acid locus may comprise genomic DNA, genomic RNA, viral DNA, viral RNA, bacterial DNA, or bacterial RNA. The target nucleic acid locus may be within a cell. The target nucleic acid locus may be in vitro. The target nucleic acid locus may be within a eukaryotic cell or a prokaryotic cell. The cell may be an animal cell, a human cell, bacterial cell, archaeal cell, or a plant cell. The enzyme may induce a single or double-stranded break at or proximal to the target locus of interest.

In cases where the target nucleic acid locus may be within a cell, the enzyme may be supplied as a nucleic acid containing an open reading frame encoding the enzyme having a HEPN domain having at least about 75% (e.g., at least about 90%, at least about 91%, at least about 92%, at least about 93%, at least about 94%, at least about 95%, at least about 96%, at least about 97%, at least about 98%, at least about 99%) identity to any one of SEQ ID NOs: 1-2. The deoxyribonucleic acid (DNA) containing an open reading frame encoding said endonuclease may comprise a sequence substantially identical to any of SEQ ID NOs: 1-2 or at variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1-2. In some cases, the nucleic acid comprises a promoter to which the open reading frame encoding the endonuclease is operably linked. The promoter may be a CMV, EF1a, SV40, PGK1, Ubc, human beta actin, CAG, TRE, or CaMKIIa promoter. The endonuclease may be supplied as a capped mRNA containing said open reading frame encoding said endonuclease. The endonuclease may be supplied as a translated polypeptide. The at least one engineered sgRNA may be supplied as deoxyribonucleic acid (DNA) containing a gene sequence encoding said at least one engineered sgRNA operably linked to a ribonucleic acid (RNA) pol III promoter. In some cases, the organism may be eukaryotic. In some cases, the organism may be fungal. In some cases, the organism may be human.

In some cases, the present disclosure may provide for an expression cassette comprising the system disclosed herein, or the nucleic acid described herein. In some cases, the expression cassette or nucleic acid may be supplied as a vector. In some cases, the expression cassette, nucleic acid, or vector may be supplied in a cell.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### EXAMPLES

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, orT
D=A, G, or T
H=A, C, orT
V=A, C, or G

### Example 1—Metagenomic Analysis for New Proteins

Metagenomic samples were collected from sediment, soil and animal. Deoxyribonucleic acid (DNA) was extracted with a Zymobiomics DNA mini-prep kit and sequenced on an Illumina HiSeq® 2500. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on documented Cas protein sequences including type VI Cas effector proteins to identify new effectors. Novel effector proteins identified by the search were aligned to documented proteins to identify potential active sites. This metagenomic workflow resulted in delineation of the MG103 and MG105 families of class II, type VI CRISPR endonucleases described herein.

### Example 2—Discovery of MG103 and MG105 Families of CRISPR Systems

Analysis of the data from the metagenomic analysis of Example 1 revealed a new cluster of undescribed putative transposase systems comprising 2 families (MG103 and MG105). The corresponding protein sequences for these new enzymes and their example subdomains are presented as SEQ ID NOs: 1-15 and 62-84.

### Example 3—Template DNA for Transcription and Translation (Prophetic)

E coli codon optimized sequences of all MG type VI nucleases are ordered (Twist Biosciences) in a plasmid with a T7 promoter and C-terminal His tag. Linear templates are amplified from the plasmids by PCR to include the T7 and nuclease sequence. crRNAs are amplified from primer pairs to include the T7 promoter, 30 nt or 20 nt spacers, and a 36 nt repeat (DR) or a reverse complement repeat (DR-RC) for in vitro transcription (Integrated DNA Technologies). Similarly, the ssRNA target is ordered as a primer pair where the forward primer contains the T7 promoter and protospacer sequences. The reverse primer contains a 15 nt complementary protospacer sequence to overlap with the forward primer and the remaining 32 nt of the ssRNA target sequence.

### Example 4—Cloning for PFS Determination Assay Targeting TetA in E. coli (Prophetic)

MGR1-1 is amplified from the Twist plasmid backbone (AmpR) with 20 nt overlapping overhangs for Gibson assembly into pMGHX (N-terminal 6xHis, MBP, NLS and C-terminal NLS). 0.02 pmol of the backbone and 0.04 pmol of the MGR1-1 ORF PCR template are assembled with NEBuilder® HiFi DNA Assembly Master Mix (New England Biolabs Inc.) at 50° C. for 15 minutes.

The TetA gene with 18 nt overlapping overhangs is then cloned into the pMGHX-MGR1-1 plasmid. 0.015 pmol of the backbone and 0.03 pmol of the TetA PCR template are assembled with NEBuilder® HiFi DNA Assembly Master Mix (New England Biolabs Inc.). All assemblies are transformed into NEB® 5-alpha Competent E. coli (High Efficiency) and confirmed by Sanger sequencing (Elim Biopharm, Inc.)

A TetA spacer library plasmid is assembled in two operations. First, a ssDNA ultramer containing a BsaI landing site comprised of 120 nt sequence with two 36 nt MGR1-1 repeats, two BsaI sites, T7 promoter and 18 nt gibson overhangs is cloned into pTCM (CmR) with a 1:1 backbone to insert molar ratio at 45° C. for 1 hour. The assembly is transformed by electroporation into Endura™ ElectroCompetent Cells (Lucigen) and confirmed by Sanger sequencing (Elim Biopharm, Inc.). Second, 1 μM of a 200 oligo spacer library (Integrated DNA Technologies) with flanking BsaI sites is made double stranded with 1 μM reverse primer, 0.1 U/μl Kelnow, 200 nM dNTPs, and 1X NEB 2.1. The reaction is heat inactivated with 0.2 mM EDTA at 75° C. for 20 minutes. The library is composed of 170 targeting and 30 non-targeting 30 nt spacers that randomly tile Tet mRNA. This library is assembled into the pTCM-BsaI-landing backbone by Golden Gate assembly with a 2:1 insert to backbone ratio at 37° C. for 1 hr then 60° C. for 5 min. pTCM-TetA-Spacer-library is transformed into NEB® Stable Competent E. coli (New England Biolabs Inc.) with >2000-fold coverage, Midiprepped (ZymoPURE™ II Plasmid Midiprep Kit) from a 75 mL culture of mixed colonies and confirmed by Sanger sequencing (Elim Biopharm, Inc.).

### Example 5—PFS Determination Assay Targeting TetA in E. coli (Prophetic)

The nuclease and spacer library plasmids described above are transformed into NEB BL21(DE3) Competent Cells, then plated on LB plates with three different conditions: 1) LB agar plates with ampicillin, tetracycline, and chloramphenicol, which allows all transformants with both plasmids to grow (positive control). 2) LB agar plates with ampicillin, chloramphenicol, IPTG, anhydrotetracycline, and fusaric acid. The addition of fusaric acid selects against expression of the tetA gene, while anhydrotetracycline induces tetA expression. Therefore, cells which knock down tetA production are favored for growth, which is accomplished via successful targeting of tetA via the nuclease and correct crRNA (selection condition). 3) LB agar plates with ampicillin, chloramphenicol, anhydrotetracycline, and fusaric acid. The addition of fusaric acid selects against expression of the tetA gene, while anhydrotetracycline induces the tetA expression. In this instance, since no IPTG is present, nuclease expression is repressed and all cell growth can be repressed by fusaric acid (negative control). All colonies in the selection condition are scraped and mini prepped. The spacers are PCR amplified, illumina primers are added, and then NGS sequenced. The resulting sequencing data enables the identification of enriched spacer sequences that successfully target tetA.

### Example 6—In Vitro Transcription and Labeling of crRNA and ssRNA Target (Prophetic)

RNA is produced by in vitro transcription using HiScribe™ T7 High Yield RNA Synthesis Kit. The ssRNA target is labeled in two ways to generate two alternate labeled substrates. It is body-labeled with 2.5 mM Fluorescein-12-UTP (Sigma Aldrich US) in the in vitro transcription reaction. Separate reactions are also 5′ end-labeled with Fluorescein Maleimide and the 5′ EndTag DNA/RNA Labeling Kit (Vector Laboratories). RNA is treated with DNAse I, incubated at 37° C. for 15 minutes, and purified using the Monarch® RNA Cleanup Kit (New England Biolabs Inc.). All transcription products are verified for yield and purity via RNA Tapestation or via a denaturing urea PAGE gel.

### Example 7—TXTL Expressions (Prophetic)

Nucleases are expressed in transcription-translation reaction mixtures using myTXTL® Sigma 70 Master Mix Kit (Arbor Biosciences). The final reaction mixtures contain 5 nM nuclease DNA template, 0.1 nM pTXTL-P70a-T7rnap and 1X of myTXTL® Sigma 70 Master Mix. The reactions are incubated at 29° C. for 16 hours then stored at 4° C.

### Example 8—PURExpress Expressions (Prophetic)

5 nM of nuclease PCR templates are expressed at 37° C. for 3 hours with PURExpress® In Vitro Protein Synthesis Kit (New England Biolabs Inc.) for cleavage with in vitro transcribed RNA. These reactions are used to test in vitro cleavage following the same procedure as described in the cleavage reactions section.

### Example 9—E. coli Expression and Purification (Prophetic)

Plasmids are transformed into BL21(DE3) Competent E. coli (New England Biolabs Inc.) and inoculated into Luria Broth medium for overnight seed cultures. The overnight cultures are then used to inoculate 500 ml Magic Media (Thermo) expression medium and the manufacturer's protocol is followed to express the protein. Cells are harvested and lysed by sonication in 20 mM Tris (Sigma T2319-100ML), 300 mM sodium chloride (VWR VWRVE529-500ML), 5% glycerol, 10 mM MgCl2, with 10 mM imidazole (Sigma 68268-100ML-F), and Pierce EDTA free protease inhibitor cocktail (Fisher PIA32965), pH 7.5. Clarified lysates are purified by nickel affinity chromatography on an Akta FPLC with a 5 ml HisTrap FF column. The final protein storage buffer comprises 50 mM Tris-HCl, 300 mM NaCl, 10 mM MgCl2, 5% glycerol; pH 7.5.

### Example 10—Cis-Cleavage Reactions (Prophetic)

### With TXTL Expression

ssRNA cleavage reactions are carried out by incubating 100-250 nM of body-labeled ssRNA target, a 5-fold dilution of the TXTL expressions, and 100-500 nM of crRNA in 10 mM TrisHCl pH 7.5, 50 mM NaCl, 0.5 mM MgCl2, 1U/μL Murine RNase inhibitor (New England Biolabs Inc.), and 0.1% BSA at 37° C. for 30 minutes. Each reaction is quenched with 0.8 U of Proteinase K (New England Biolabs Inc.) for 15 min at 37° C. then mixed equal parts of RNA loading dye, denatured at 95° C. for 5 min, and then cooled on ice for 2 min. Cleavage products are analyzed by denaturing gel electrophoresis on 15% PAGE TBE-Urea gels.

### With PURExpress Expression

500 nM crRNA and a 5-fold dilution of PURExpressed nuclease are incubated at 37° C. for 15 minutes. Following the pre-incubation of crRNA and nuclease 250 nM of ssRNA target, 10 mM TrisHCl pH 7.5, 50 mM NaCl, 0.5 mM MgCl2, 1U/μL Murine RNase inhibitor (New England Biolabs Inc.), and 0.1% BSA at 37° C. for 30-60 minutes at 37° C. Each reaction is quenched with 0.8 U of Proteinase K (New England Biolabs Inc.) for 15 min at 37° C. then mixed equal parts of RNA loading dye, denatured at 95° C. for 5 min, and then cooled on ice for 2 min. Products are analyzed as described above.

### With IMAC Purified Nucleases

400 nM crRNA and 400 nM purified nuclease are incubated at 37° C. for 15 minutes. Following the pre-incubation of crRNA and nuclease 200 nM of ssRNA (5′end-labeled or body-labeled RNA) target, 50 mM NaCl, 10 mM Tris-HCl, 10 mM MgCl2, 100 pg/ml BSA pH 7.9, and 1U/μL Murine RNase inhibitor (New England Biolabs Inc.) at 37° C. for 30-60 minutes at 37° C. Each reaction is quenched with 0.8 U of Proteinase K (New England Biolabs Inc.) for 15 min at 37° C. then mixed equal parts of RNA loading dye, denatured at 95° C. for 5 min, and then cooled on ice for 2 min. Products are analyzed as described above.

crRNA mediated ssRNA cleavage by these nucleases results in multiple products, in patterns dependent on the structure and sequence of the RNA target. Positive cleavage also decreases the signal of the 66 nt ssRNA target relative to uncleaved.

### Example 11—PURExpress Activity Via GFP Reporter Targeting (Prophetic)

5 nM of nuclease PCR templates are expressed at 37° C. for 30 minutes with PURExpress® In Vitro Protein Synthesis Kit (New England Biolabs Inc.). After 30 minutes, the reaction is split and supplemented with 50-100 nM in vitro transcribed RNA and the mRNA for GFP. Fluorescence is followed in 384-well format in a fluorescent plate reader (Synergy HTX). Relative activity is detected via reduction of fluorescence in the presence of a targeting vs non-targeting spacer. This assay can also be modified to report on trans-cleavage activity (rather than combined cis and trans) via addition of a non-fluorescent targeted gene (e.g. DHFR). In this case, reduction in GFP occurs if trans cleavage is activated by the correct targeting of the non-fluorescent gene.

### Example 12—RNA Cleavage in Mammalian Cells (Prophetic)

A reporter HEK293T cell line is built expressing enhanced GFP (eGFP) with a C terminal PEST tag to promote protein instability (ueGFP) under the human phosphoglycerate kinase 1 promoter (hPGK). Type VI nucleases candidates are human codon optimized and cloned into a lentiviral vector under the EF1a promoter. gRNAs for the Type VI nucleases are cloned under a U6 promoter in a separate lentiviral vector. Cells successfully transduced with both the Type VI nuclease and the gRNA are selected via double selection with 1 pg/mL puromycin and 5 pg/mL of blasticidin for 3 days. GFP signal is analyzed by flow cytometry. GFP mRNA is extracted using mirVANA RNA extraction kit and quantified using qPCR. Successful Type VI candidates show >50% loss of signal of GFP when quantified via flow cytometry and qPCR.

### Example 13—Nuclease Discovery and Characterization in the MG103 Family

### In Silico Identification of Novel Compact Class 2, Type VI Nucleases in the MG103family

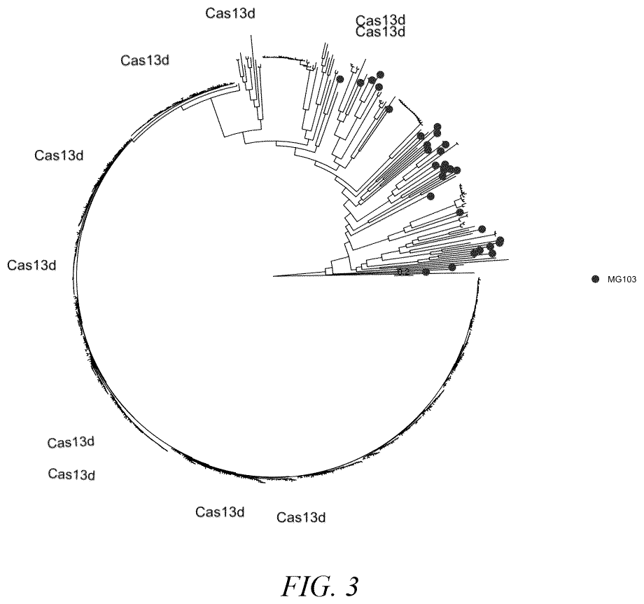

Type VI nucleases were searched in an extensive database of assembled microbial, eukaryotic, and viral genomes using hmmsearch (http://hmmer.org/). Type VI homologs were dereplicated at 99% amino acid identity (AAI) to remove redundancy using MMseqs2 (easy-cluster —cov-mode 1-c 0.8; Nature biotechnology 2017, 35 (11), 1026-1028). After dereplication, 1,283 cas13 proteins and 205 reference sequences were globally aligned with MAFFT (mafft —large-globalpair; Molecular biology and evolution 2013, 30 (4), 772-780), and a phylogenetic tree was constructed using FastTree (PloS one 2010, 5 (3), e9490) with default parameters. Novel Type VI nucleases (FIG. 3, SEQ ID NOs: 62-84) were identified based on the tree's topology given the presence of known references.

### In Vitro RNA Nuclease Activity Demonstrated by GFP Fluorescence Assay

### DNA Templates for In Vitro Transcription and Translation of Class 2, Type VI Systems

Minimal array eBlocks were designed with a T7 promoter, one 36 bp repeat, one 30 bp spacer targeting the deGFP mRNA, followed by a second identical repeat sequence and a 21 bp primer binding site (IDT) (SEQ ID NOs: 18-61). To extend the sequence length to 300 bp, minimal arrays carried an additional 159 bp 5′ end sequence upstream of the T7 promoter. In a second design, the repeat orientations in the minimal arrays were reversed. In a third design, a spacer sequence not targeting the deGFP mRNA was included. A fourth design carried a 30 bp spacer sequence complementary to a 101 nt activator RNA substrate.

E. coli codon-optimized nuclease plasmids were obtained from Twist Bioscience. Linear nuclease templates and minimal array templates were amplified by PCR, cleaned, concentrated with HighPrep™ PCR Clean-up System (MagBioGenomics), and eluted in 10 mM Tris HCl pH 8.0. PCR templates were verified for yield and purity by Nanodrop and D1000 Tapestation (Agilent Technologie).

### RNA Templates for In Vitro Transcription of Type VI Systems

A deGFP linear template containing T7 promoter, deGFP gene, and T7 terminator was amplified from a T7p14_deGFP plasmid from ArborBioscences (SEQ ID NO: 16). The amplicon was cleaned and concentrated with HighPrep™ PCR Clean-up System (MagBioGenomics) and eluted in RNase-free water. deGFP mRNA was synthesized with HiScribe™ T7 High Yield RNA Synthesis Kit and cleaned with Monarch® RNA Cleanup Kit (50 pg) (New England Biolabs Inc.). Transcription products were verified for yield and purity by Nanodrop and RNA Tapestation (Agilent Technologies).

To test in trans cleavage activity of type VI enzymes on collateral RNA targets, a second substrate template was designed. A ssDNA sequence in reverse complement was ordered with a T7 promoter and a 100 nt sequence with a 30 nt targetable sequence (SEQ ID NO: 17). An 18 nt complementary sequence to the T7 promoter was annealed to the ssDNA oligo and synthesized as described above.

### In Vitro Fluorescence-Based RNA Cleavage Assay

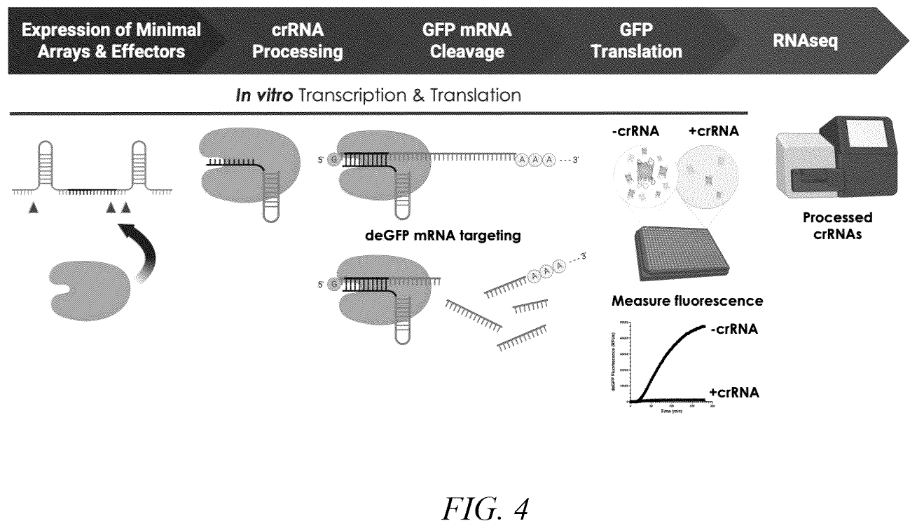

Cleavage was conducted in 20 μL reactions with PURExpress® In Vitro Protein Synthesis Kits (NEB Inc.). 25 nM minimal array DNA templates and 5 nM effectors DNA templates were transcribed and translated to minimal array RNA and protein at 37° C. for 20 minutes. 500 nM deGFP RNA templates were then added to each reaction as the targeting substrate. These samples were transferred to 384 black plates and sealed with ABsolute qPCR Plate Seals (Thermo Scientific), and fluorescence measurements were immediately commenced in a Synergy Neo2 multimode reader (BioTek Instruments) (FIG. 4). Measurements at 485/20 excitation and 528/20 emission were taken for 3 hours at 3 minute intervals at 37° C. Targeting of deGFP mRNA by these nucleases results in cleavage of the mRNA and translation knock down of the GFP protein that is measured as a decrease in fluorescence (RFUs). Data was plotted in RFUs vs. Time and each curve was fit to a plateau followed by one phase exponential decay.

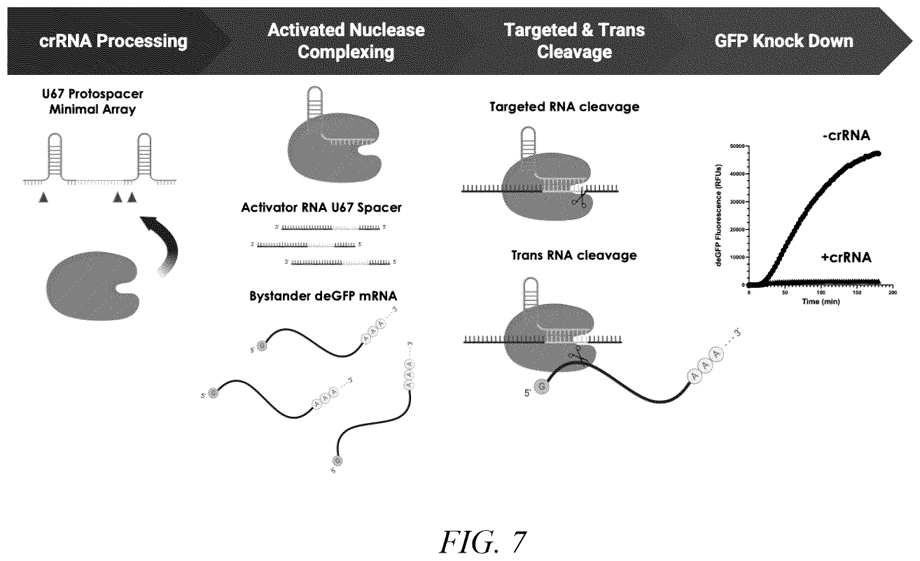

Trans-cleavage evaluation was executed as described above with different minimal array templates and targeting substrate (FIG. 7). Minimal arrays targeted a 30 nt sequence complementary to a 101 nt activator RNA target. 500 nM of the activator RNA target and deGFP mRNA bystander target were added to the reactions simultaneously. Targeting of activator RNA resulted in trans-cleavage of the deGFP mRNA and translation knock down of the deGFP protein that in turn was measured as a decrease in fluorescence (RFUs). Data was plotted in RFUs vs. Time and each curve was fit to a plateau followed by one phase exponential decay.

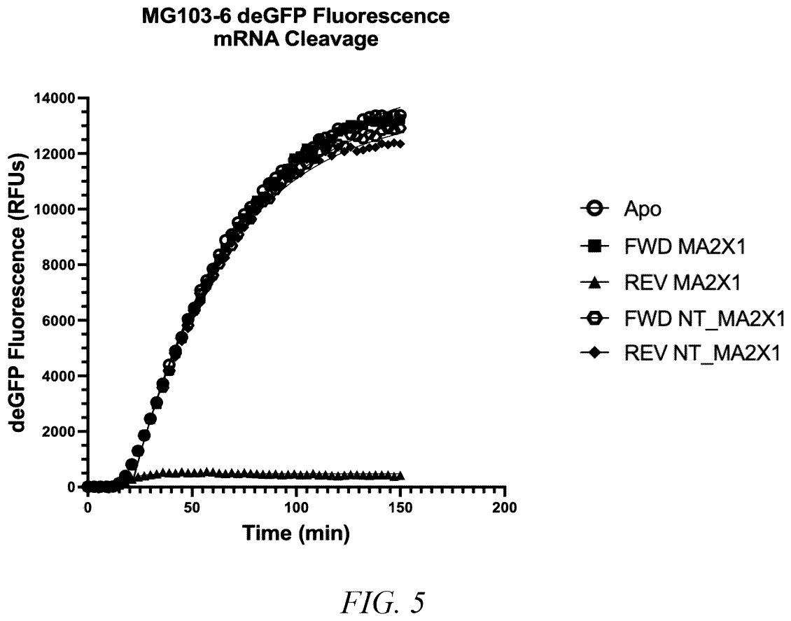

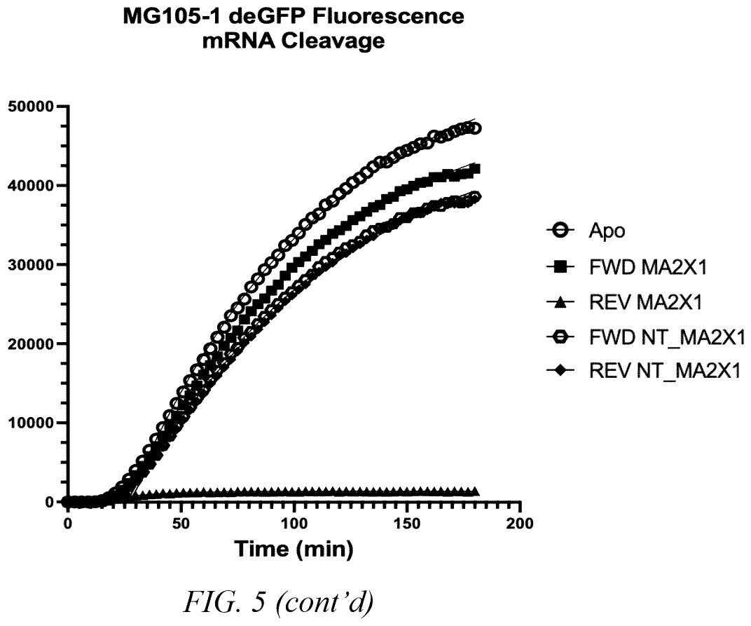

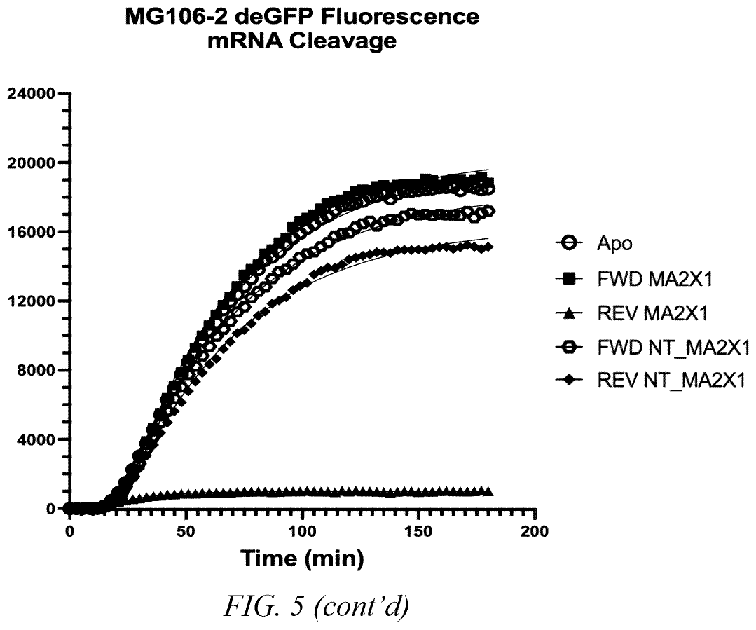

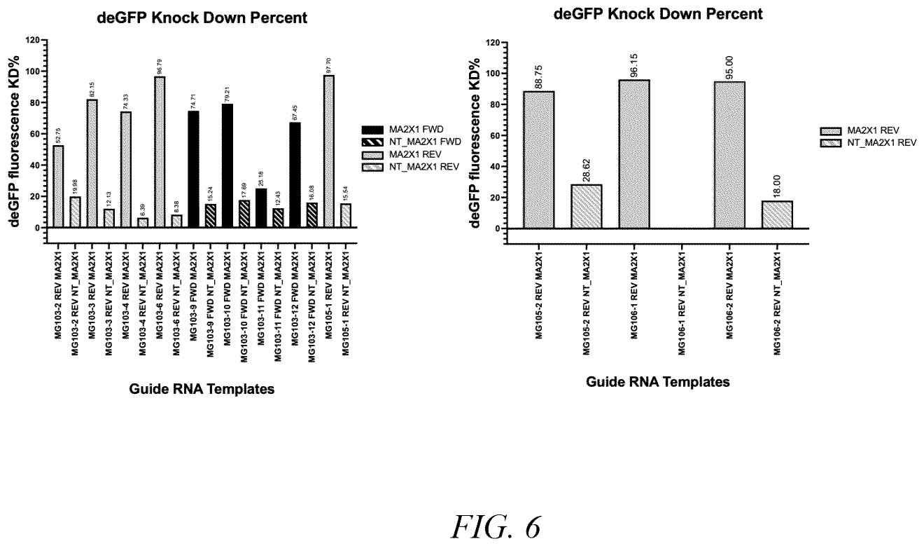

In all reactions, a lag was observed in the fluorescence signal, likely due to the time needed to translate and fold deGFP. Control reactions including Apo and non-targeting arrays translated the most deGFP and produced the most fluorescence signal. Some non-targeting minimal array reactions exhibited a slightly lower signal than Apo; this can be accounted for by transcription/translation resources being limiting when more was added to the reaction. Targeting arrays lower the fluorescence signal more than non-targeting arrays. Each data point was first subtracted from the background signal from a control reaction that did not transcribe/translate deGFP or any other templates. Knock down percentages were quantified by fitting each curve to a plateau followed by one phase exponential decay (FIG. 5). The parameter used for quantification was the plateau, which is understood to represent the max fluorescence. The Apo plateau value was subtracted from each condition then divided by the apo plateau and multiplied by 100. MG103s targeted cis-cleavage resulted in robust fluorescence knock down percents up to 96.79% (FIG. 6). Most of the active repeats (except for MG103-4) carried the AAAC-3′ motif.

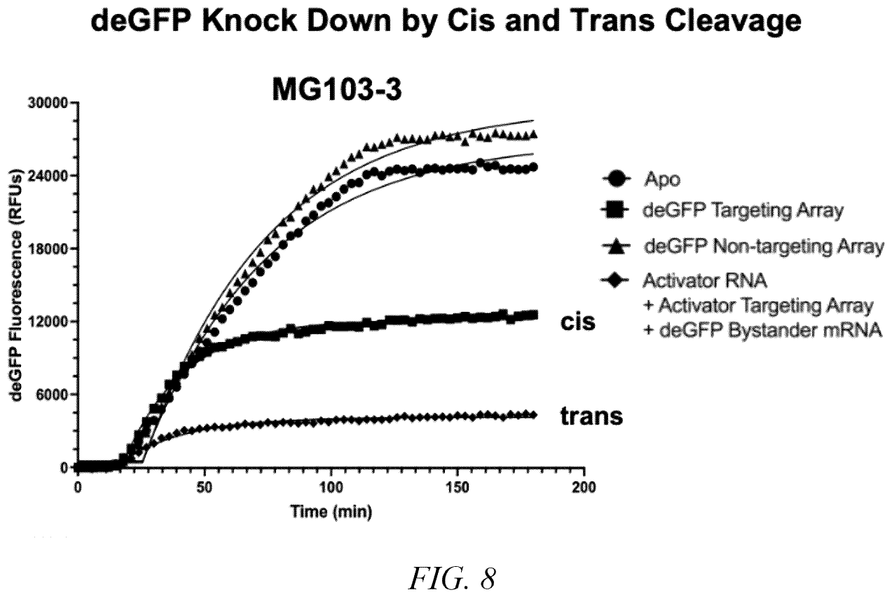

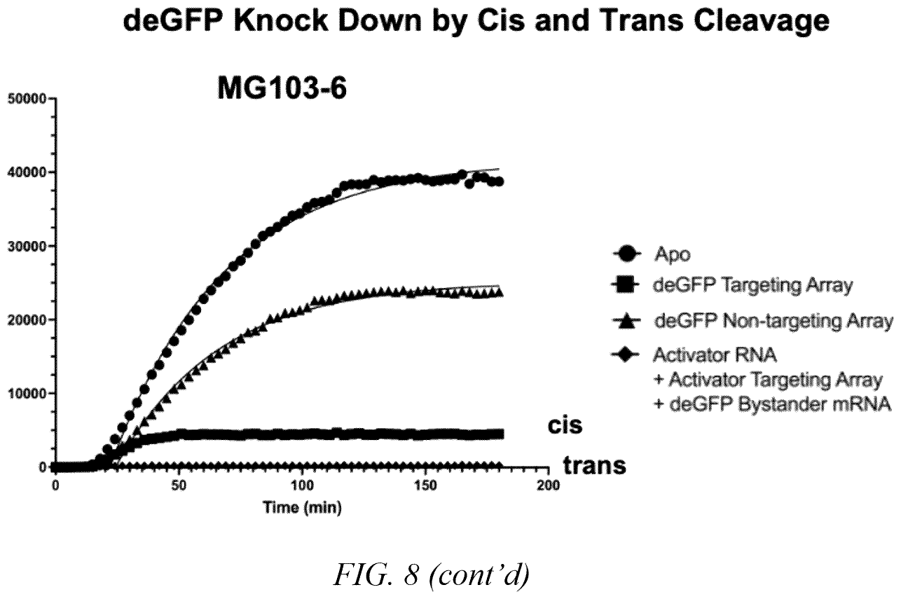

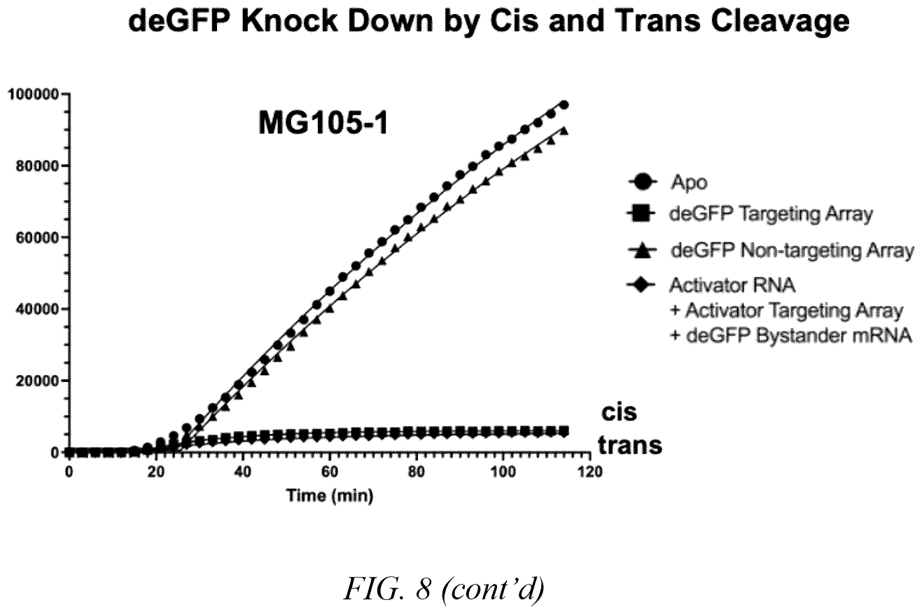

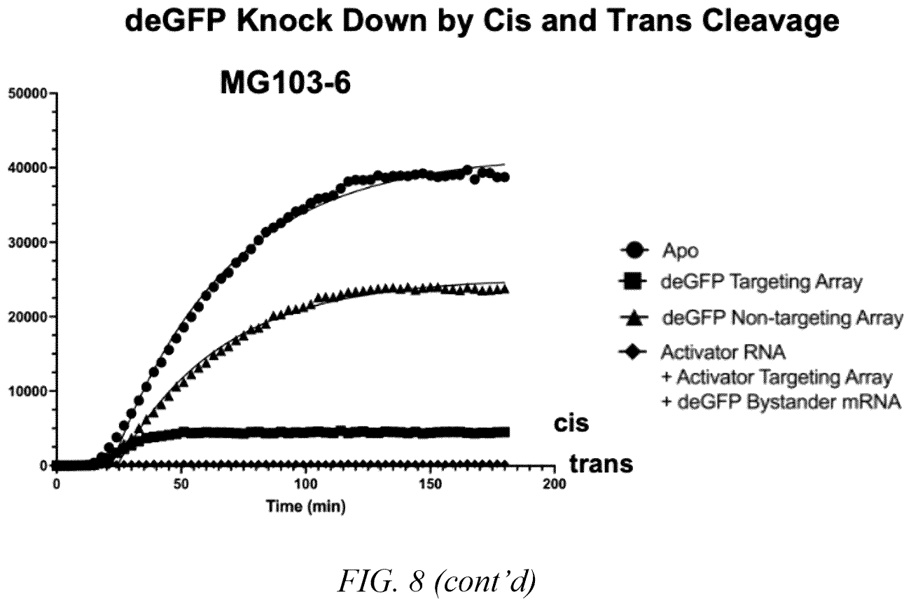

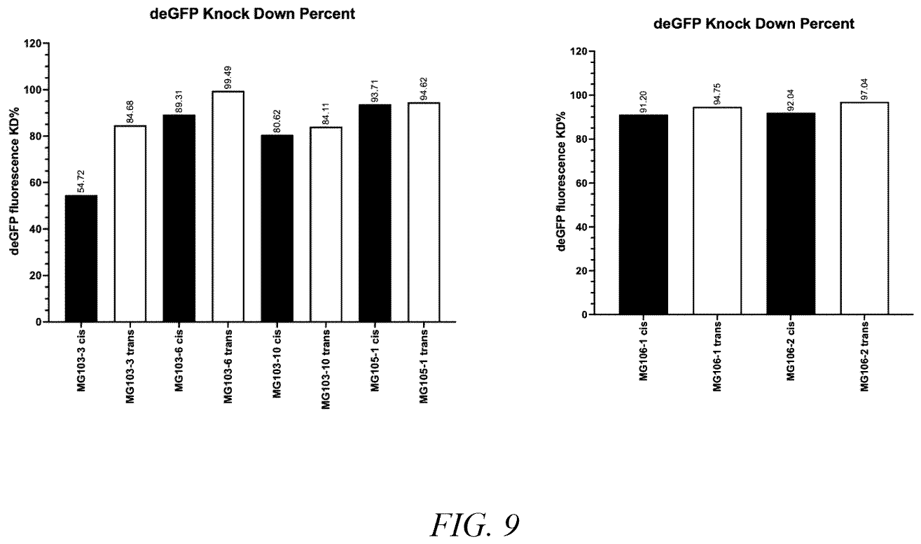

MG103 trans cleavage data was processed and analyzed as described above (FIG. 8). Cis and trans-cleavage was tested on the same day for comparison. deGFP knock down revealed comparable cleavage by both cis and trans activity (FIG. 9).

RNAseq of Processed crRNA

RNA was extracted from PURExpress cell lysate expressions following the Quick-RNA™ Miniprep Kit (Zymo Research) and eluted in 30-50 μL of water. 5′ ends of the processed crRNA were mono-phosphorylated with 10 units of T4 Polynucleotide Kinase, 40 units of Murine RNase Inhibitor, and 1X of T4 DNA Ligase Buffer (NEB Inc.) in 25-50 μL reactions. Following a 30-minute incubation at 37° C., reactions were stopped with column purification using Monarch® RNA Cleanup Kit (50 pg) (NEB Inc.). The total concentration of the transcripts were measured on a Nanodrop, Tapestation, and Qubit.

100ng-1pg of total RNA from each sample were prepped for RNA sequencing using the NEBNext Small RNA Library Prep Set for Illumina (NEB Inc.). Amplicons between 150-300 bp were quantified by Tapestation and Qubit and pooled to a concentration of 4 nM. A concentration of 12.5 μM was loaded into a MiSeq V3 kit and sequenced in a Miseq system (Illumina) for 176 total cycles. The RNAseq reads were used to identify the processed crRNA sequences. Illumina adapters were removed from all reads using fastp (see e.g., Bioinformatics 2018, 34 (17), i884-i890, which is incorporated by reference herein in its entirety). Trimmed reads were mapped to the RNA templates using BWA-MEM (See e.g., Li H. Aligning sequence reads, clone sequences and assembly contigs with BWA-MEM. 2013, Preprint Vol. 00 no. 00 2013 Pages 1-3, which is incorporated by reference herein in its entirety), and using samtools all reverse reads, unmapped reads, and reads mapping to the 5′ PCR adapter were removed.

crRNA Processing Determined by RNAseq

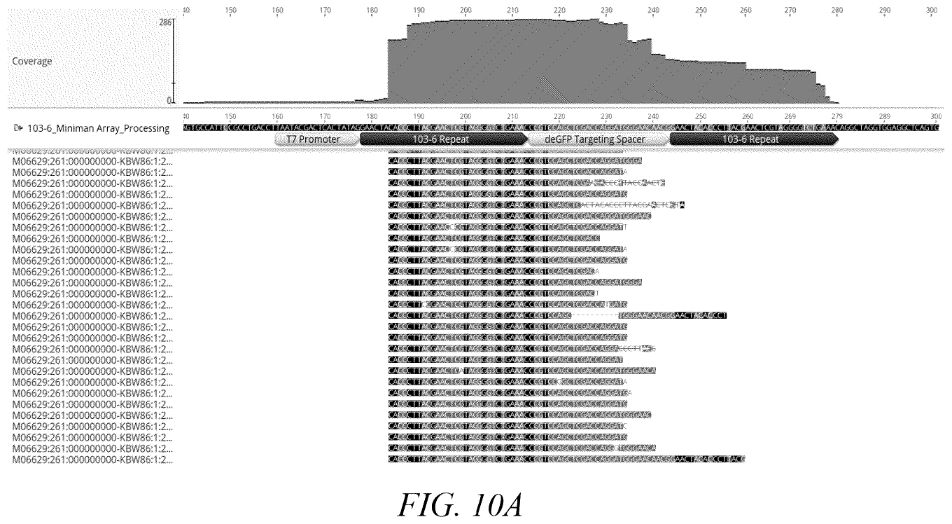

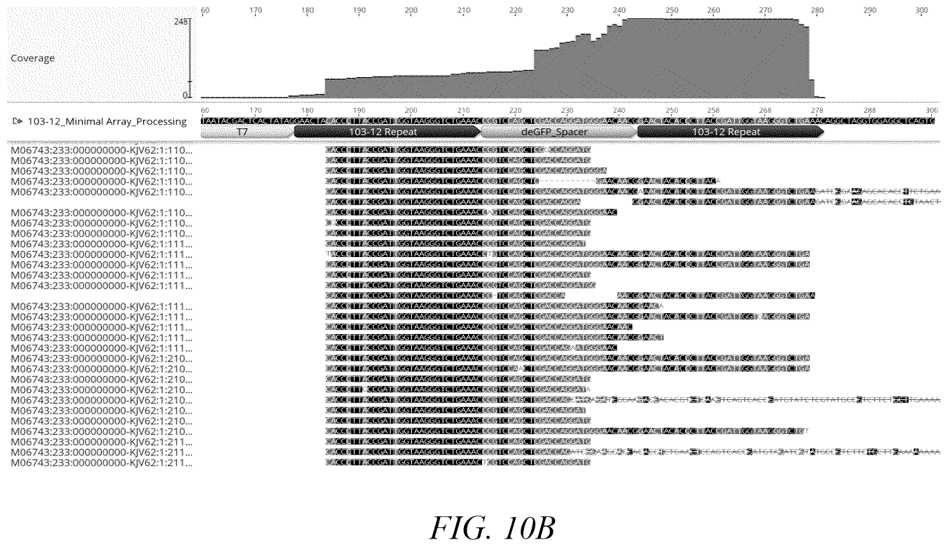

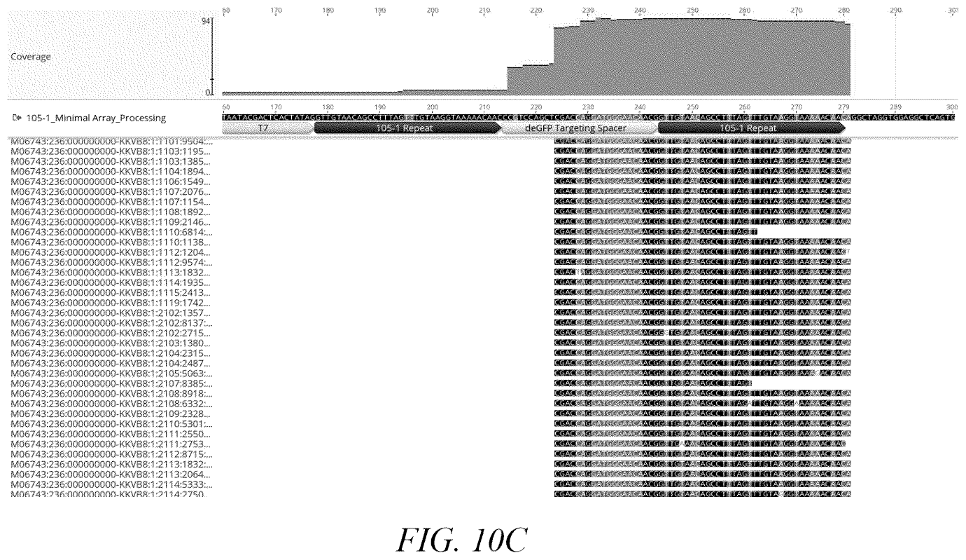

Reads mapping to the active MG103-6 and MG103-12 minimal array show processing of 6 nucleotides on the 5′ end of the repeats leaving 30 nucleotide processed repeats (FIGS. 10A and 10B). MG103-6 reads also show that the 5′ end processing trims the spacer down to 21 and 26 nt (SEQ ID NO: 36-37). MG103-12 mapped reads show some 5′ end trimming resulting in 21 nt spacer (SEQ ID NO: 55). These findings can be used to design synthetic guide RNAs by using the detected crRNA processing boundaries in designed crRNAs.

### Confirmation of Processed Sites (Prophetic)

ssDNA oligo templates of RNAseq-confirmed processed crRNA are designed with a T7 promoter upstream of the crRNA sequence and ordered as reverse complements. An 18 nt complementary sequence to the T7 promoter is annealed to each ssDNA oligo and synthesized as described above. To validate activity of the processed crRNA designs, the same in vitro fluorescence based RNA cleavage assay is performed.

### RNA Cleavage Activity in Mammalian Cells

Lentiviruses were used to create a reporter HEK293T cell line expressing (CMV promoter) enhanced GFP (eGFP) with a C-terminal PEST tag to promote protein instability (see e.g., Science 1986, 234 (4774), 364-368, which is incorporated by reference herein in its entirety) (ueGFP, SEQ ID NO: 85) and enhance the turnover rate of GFP to make enzyme fluorescence more responsive to changes in mRNA levels. The ueGFP engineered cell line was used as a reporter. The spacers of each type VI CRISPR enzyme were designed to target the 5′ of the ueGFP mRNA, thus knocking down the GFP fluorescence.

Selected type VI nuclease candidates were human codon-optimized and cloned into a mammalian expression vector under CMV promoter (MG103-2, MG103-3, MG103-6, MG103-7, MG103-9, MG103-10, MG103-11, MG103-12, MG103-14, and the positive control; SEQ ID NO: 126-134). CRISPR arrays containing the predicted repeat and 30 nt targeting spacers comprising 5 repeats and 4 spacers (SEQ ID NOs: 106-113) were cloned into an expression vector under a U6 promoter. Moreover, CRISPR arrays comprising 3 repeats and 2 spacers were chemically synthesized (IDT) with 2′-O-Methyls and phosphorothioate (PS) bonds at the 5′ and 3′ ends (3 2′-O-Methyls and 3 PS bonds in each end) (SEQ ID NOs: 90-105 and 135-154).

ueGFP-expressing cells were transfected with plasmids containing the effector alone (Apo condition) as a control, or with either plasmid-encoded CRISPR arrays or chemically synthesized CRISPR arrays. Plasmid DNA was transfected using Lipofectamine 2000 and chemically synthesized arrays were transfected using Lipofectamine Messenger Max. Briefly, 150,000 cells were seeded into 24 well plates. 750 ng of plasmid containing the effector and 500 ng of plasmid containing the CRISPR array were mixed in serum-free Optimem. In parallel, Optimem was mixed with 2 μL of lipofectamine 2000 (per reaction and pooled as needed).

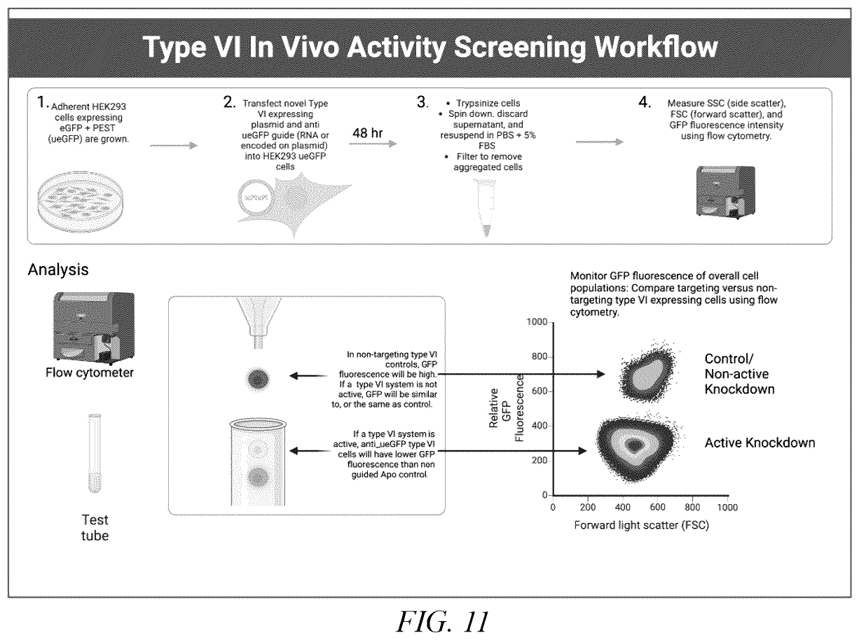

Plasmids in Optimem and Lipofectamine 2000 in Optimem were incubated separately for 5 minutes and then mixed and vortexed together, followed by a 30-minute incubation. When the chemically synthesized array was used instead, 10 pmoles of chemically synthesized guide was mixed with Optimem. Separately, Optimem was mixed with 1.5 μL of Lipofectamine messenger max. Each reaction was incubated for 5 minutes, then mixed together and incubated for an extra 15 minutes. The lipid/nucleic acid mixture was then added to the seeded cells. 48 hours post transfection, cells were trypsinized, pelleted at 300 g for 10 minutes, resuspended in 300 μL of PBS with 5% FBS, and filtered through a 0.4 μM mesh in order to filter out doublets, or higher cellular aggregations. Single cells were then analyzed by flow cytometry (cartoon depicting the process shown in FIG. 11).

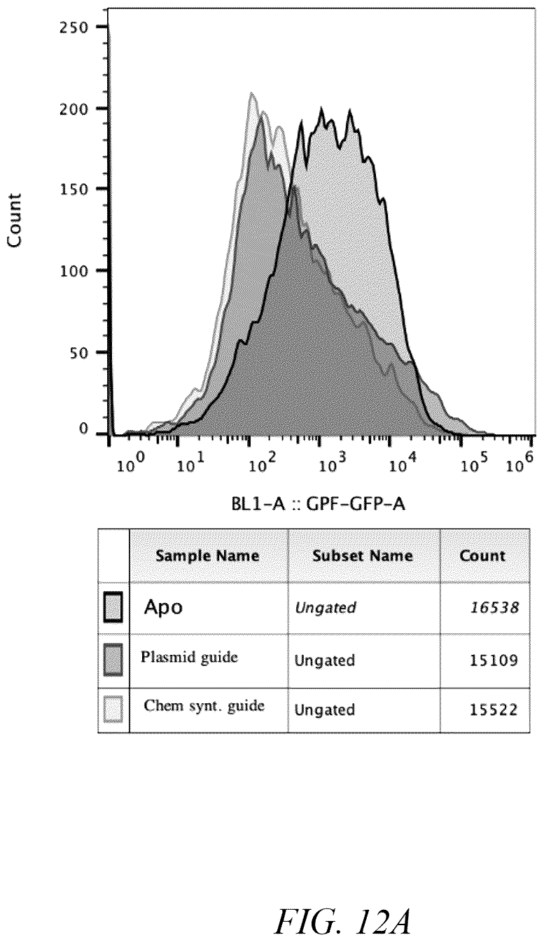

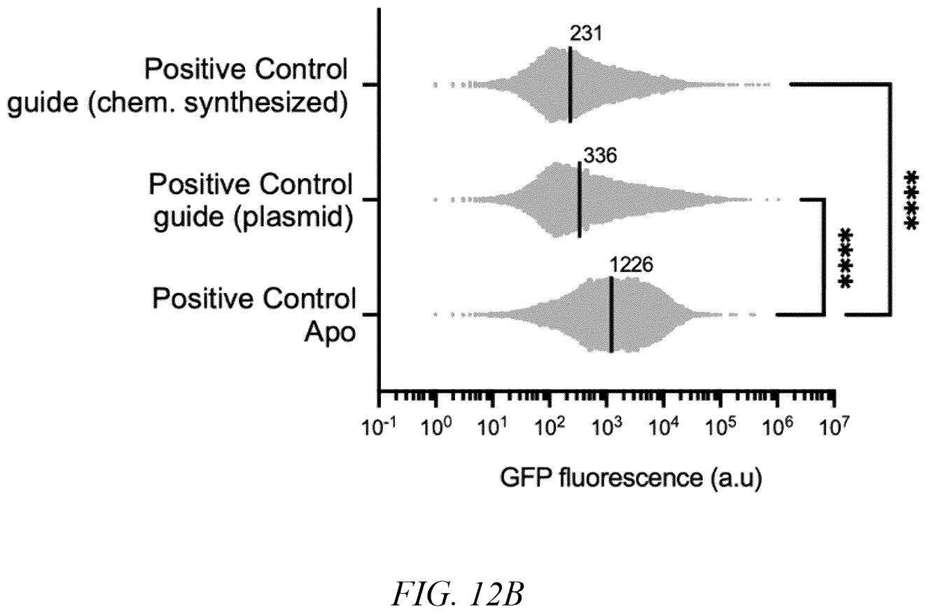

In order to validate the suitability of the ueGFP cell line, along with the experimental design, positive controls were run, along with a spacer array targeting ueGFP encoded in a plasmid or as chemically synthesized guides. The suitability of the experimental setup was validated by observing a considerable knock-down of GFP fluorescence in the conditions when CRISPR arrays were present (FIG. 12A). The quantification of FIG. 12A is shown in FIG. 12B.

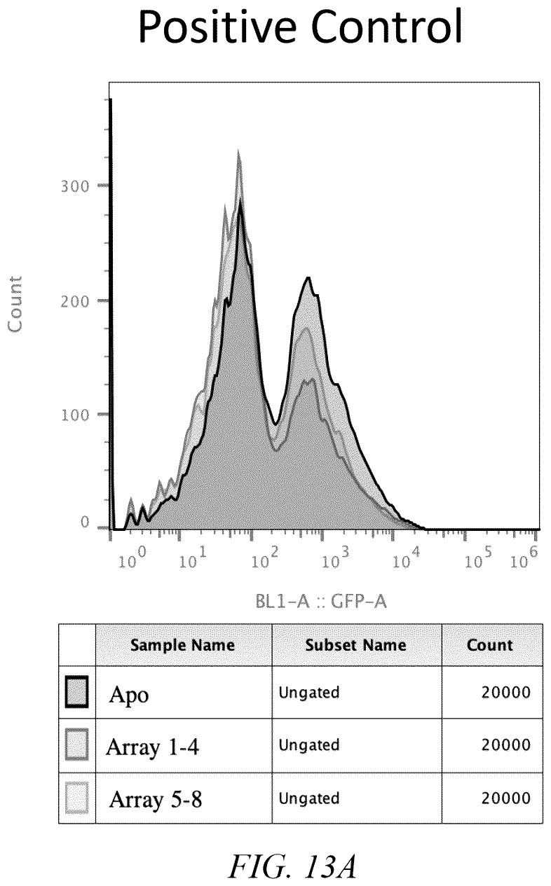

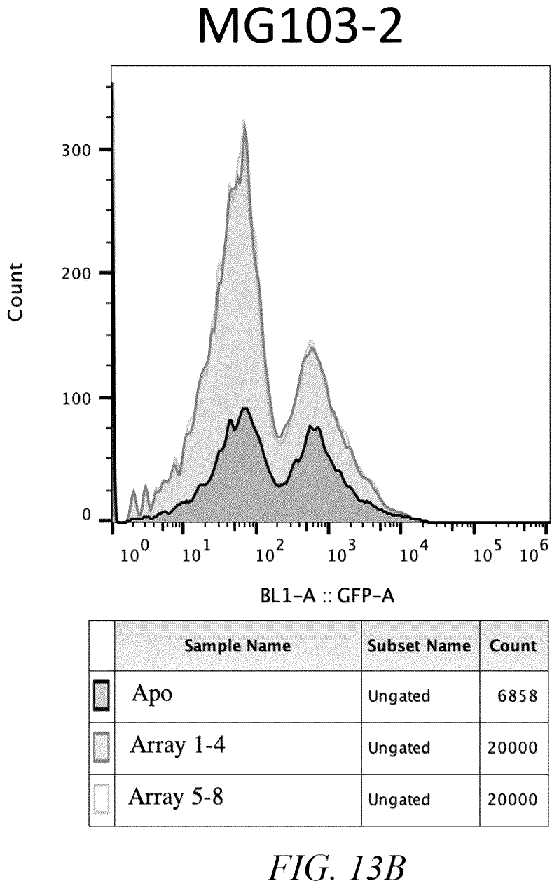

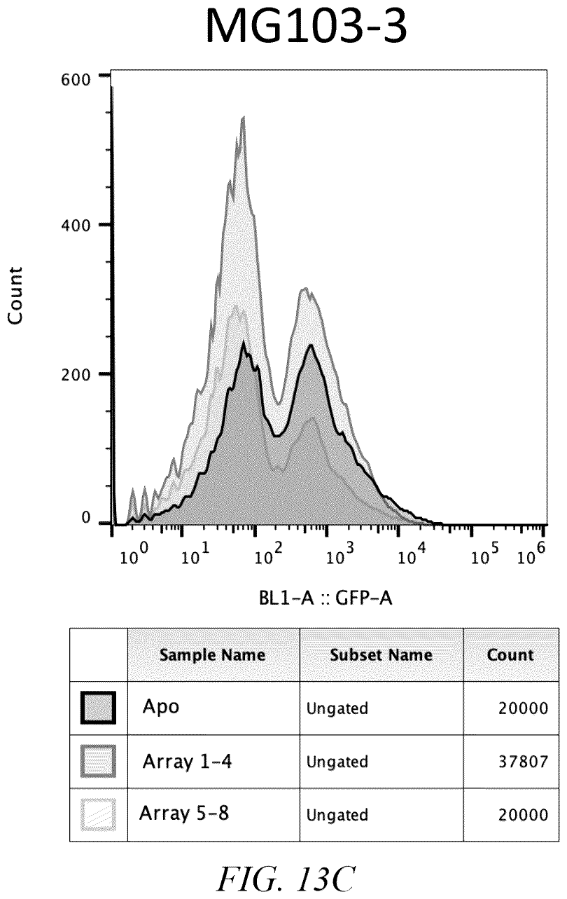

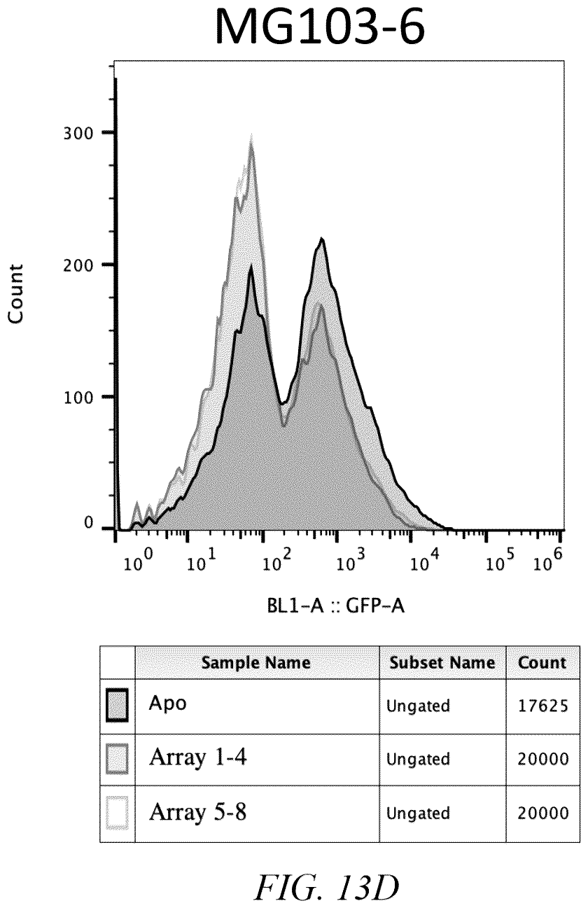

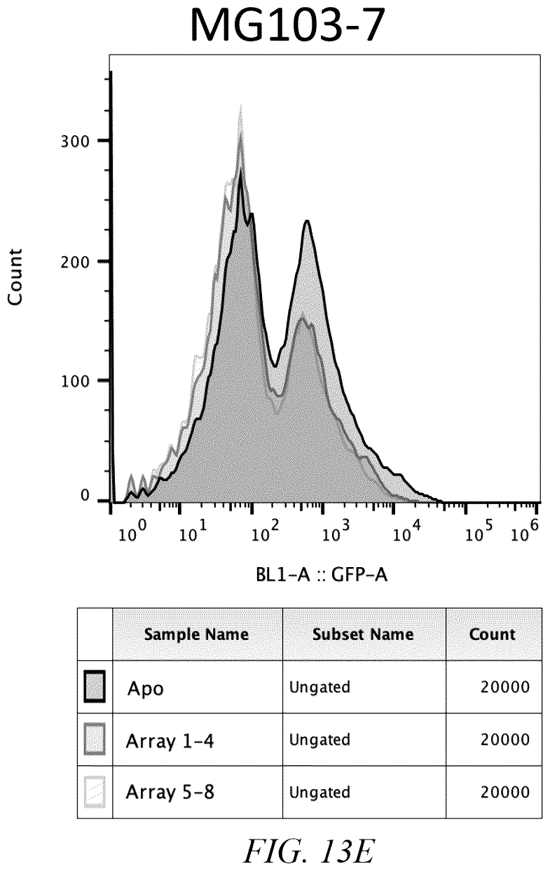

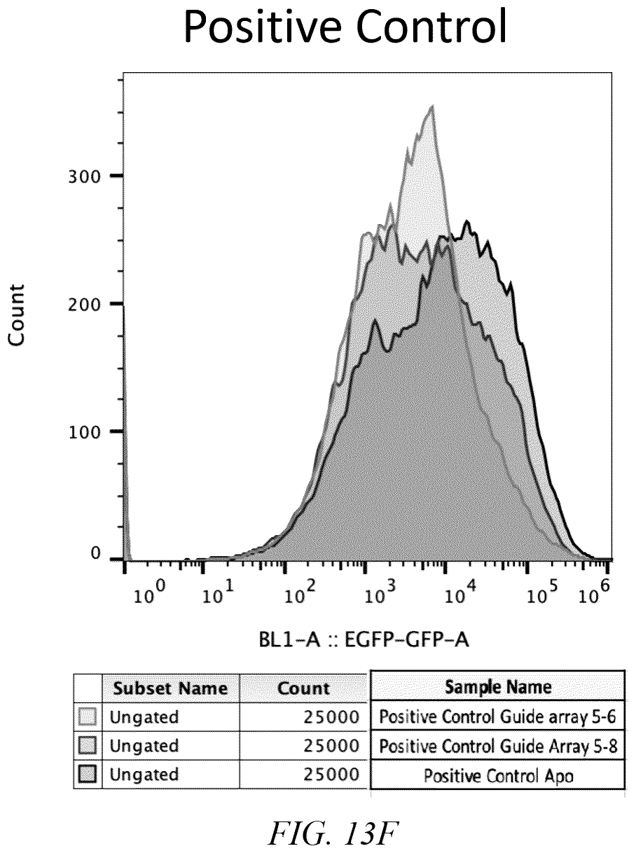

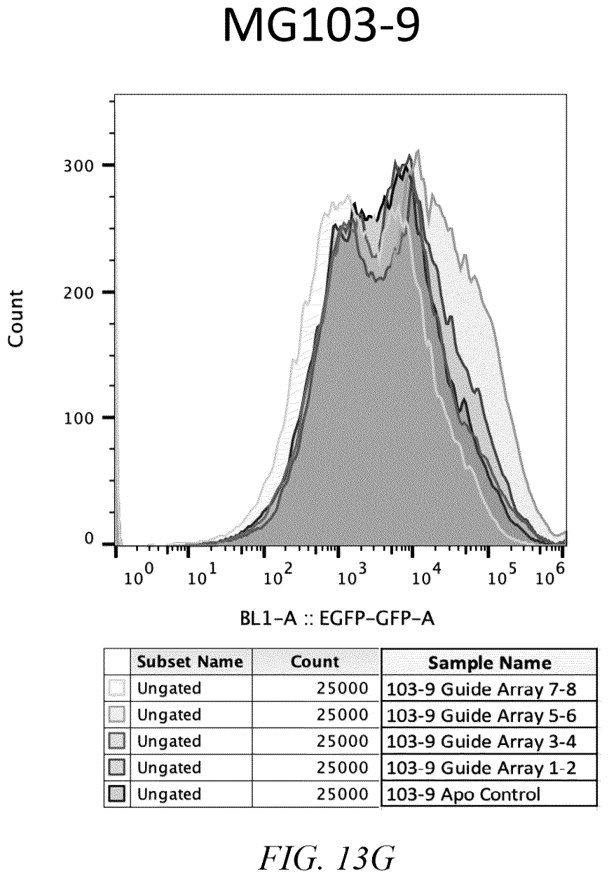

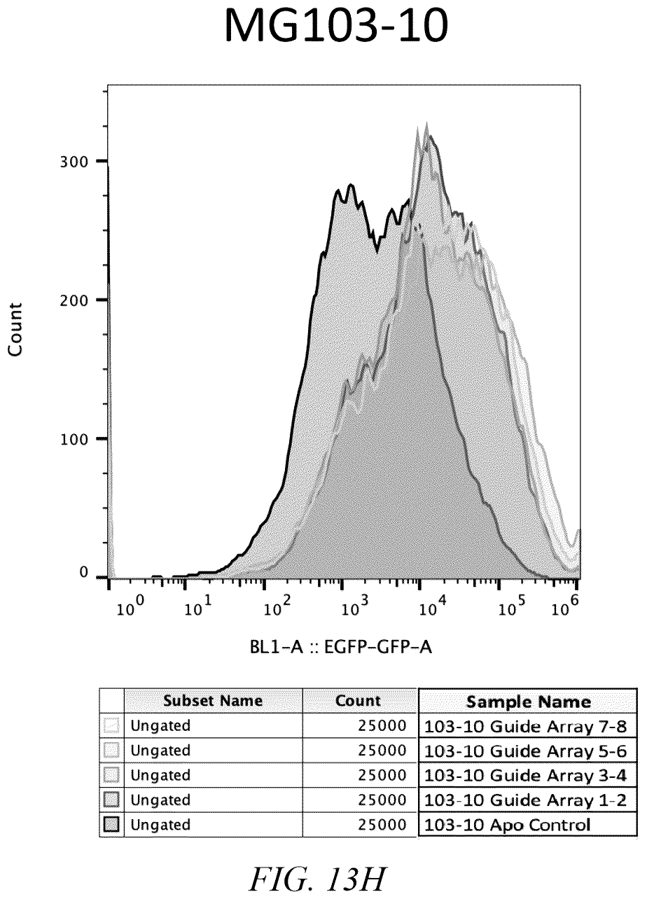

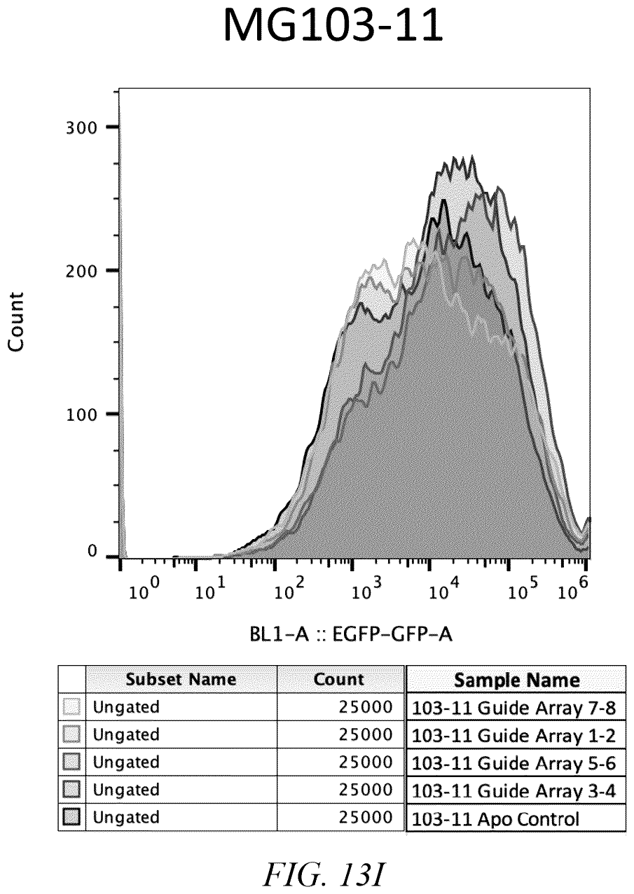

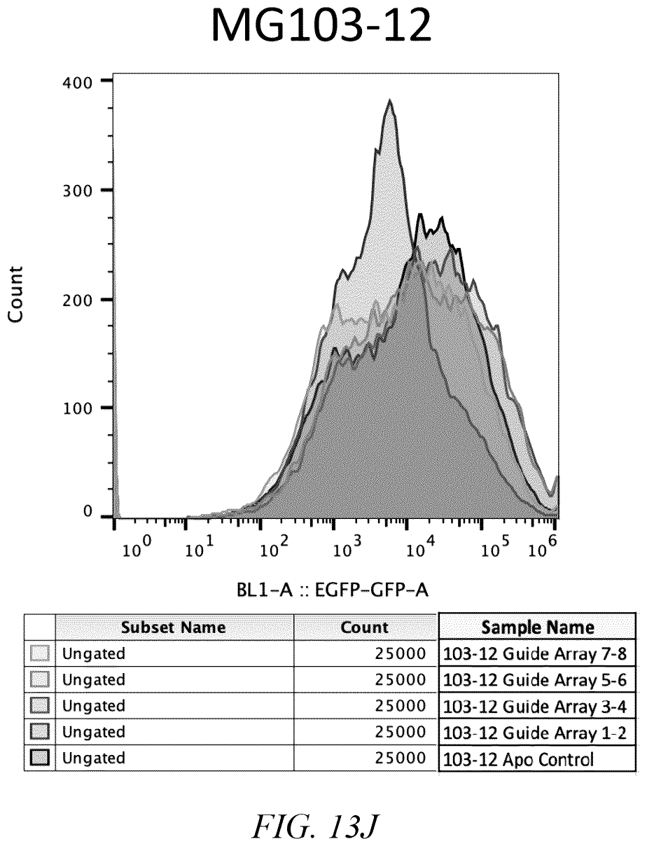

Once the system was validated, several MG103 nucleases were tested: MG103-2, MG103-3, MG103-6, MG103-7, MG103-9, MG103-10, MG103-11, MG103-12, and MG103-14, along with the positive control. Since validation using gRNAs encoded in a plasmid worked to similar levels to chemically synthesized arrays (FIG. 12B), plasmid-encoded guides were tested for the MG103-2, MG103-3, MG103-6 and MG103-7 systems, and chemically synthesized guides were tested for MG103-9, MG103-10, MG103-11, MG103-12, and MG103-14. As shown in FIGS. 13A-13J, FIGS. 14A-14K, and FIGS. 15A-15B, there were various levels of GFP knockdown in guided vs. unguided conditions across all novel nucleases tested.

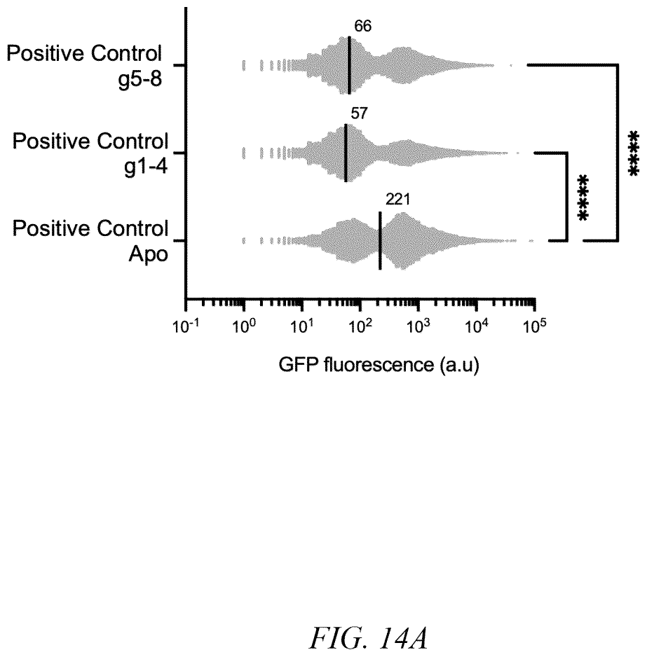

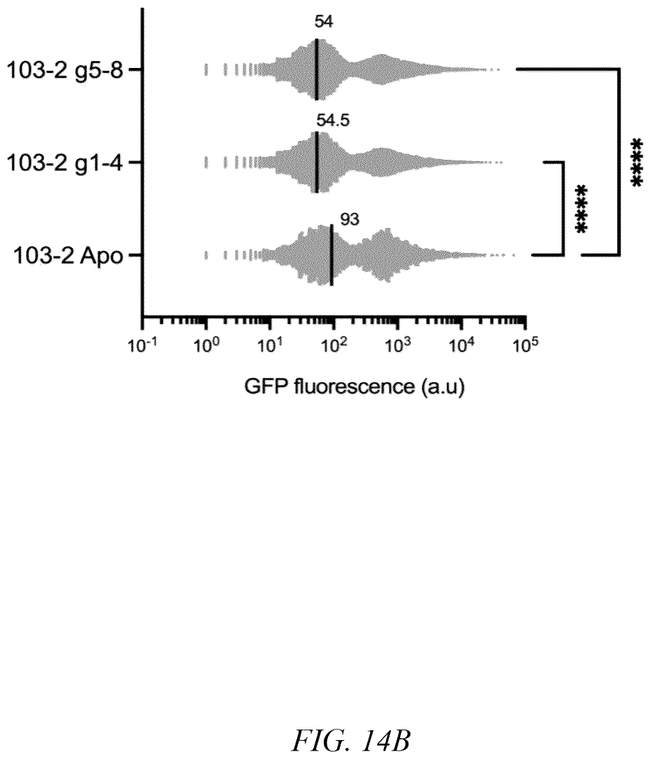

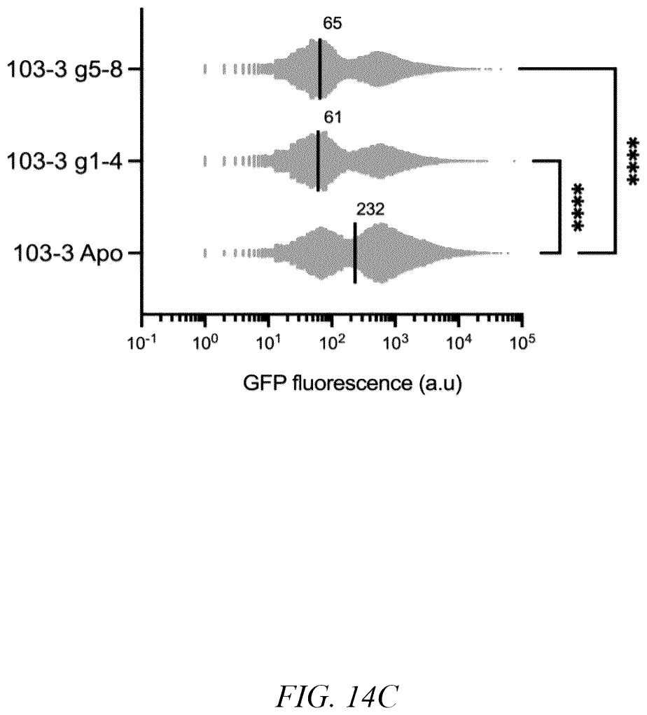

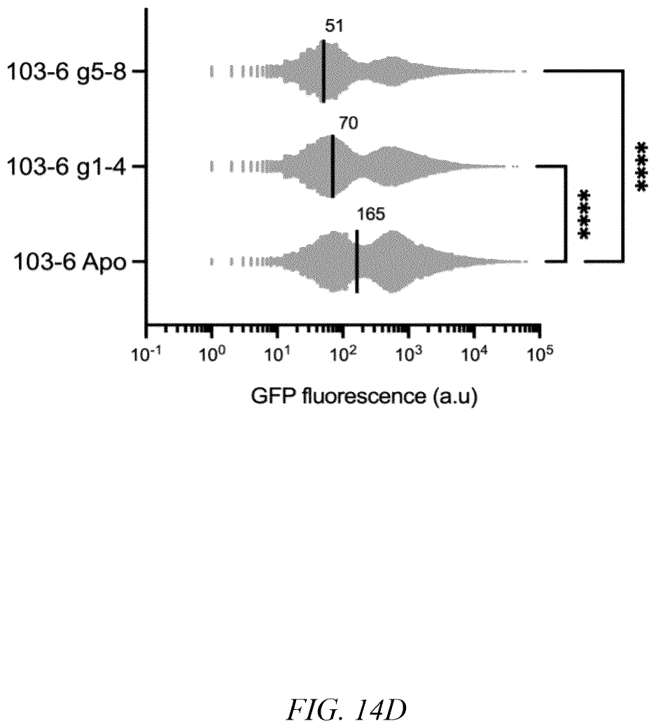

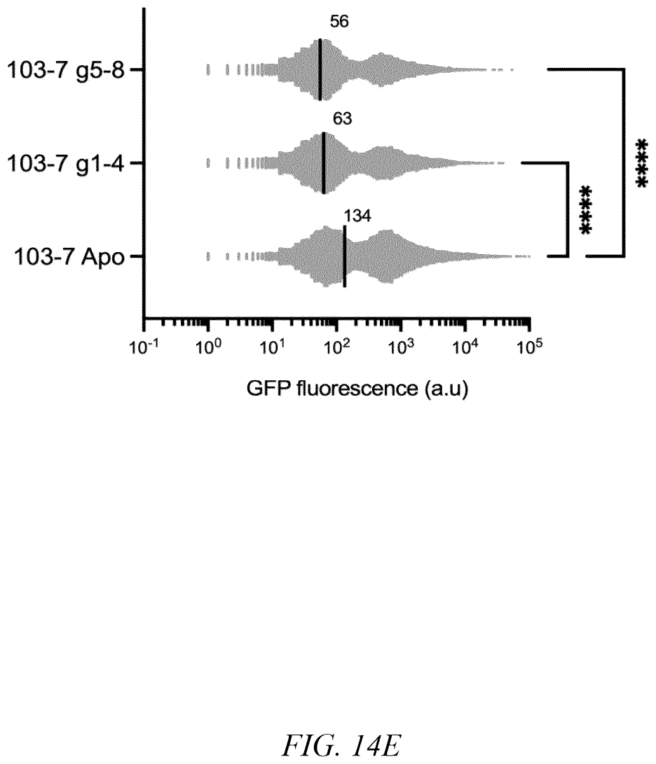

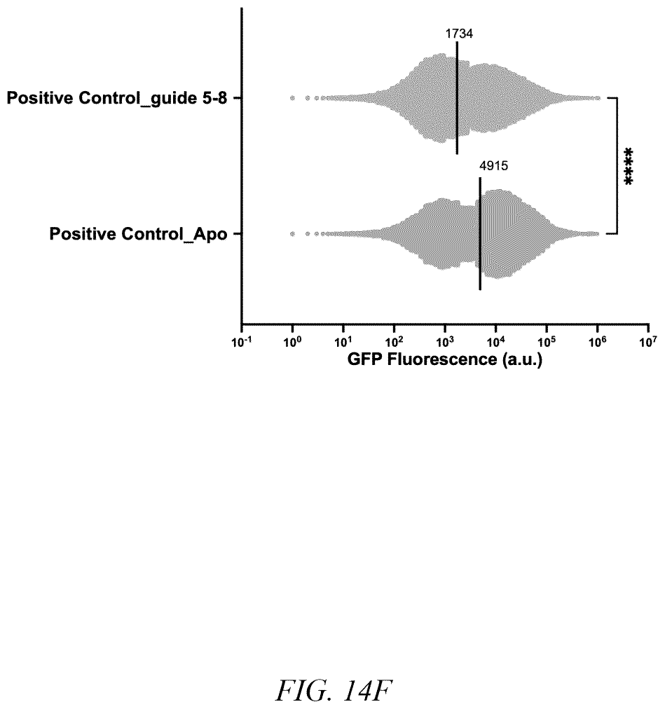

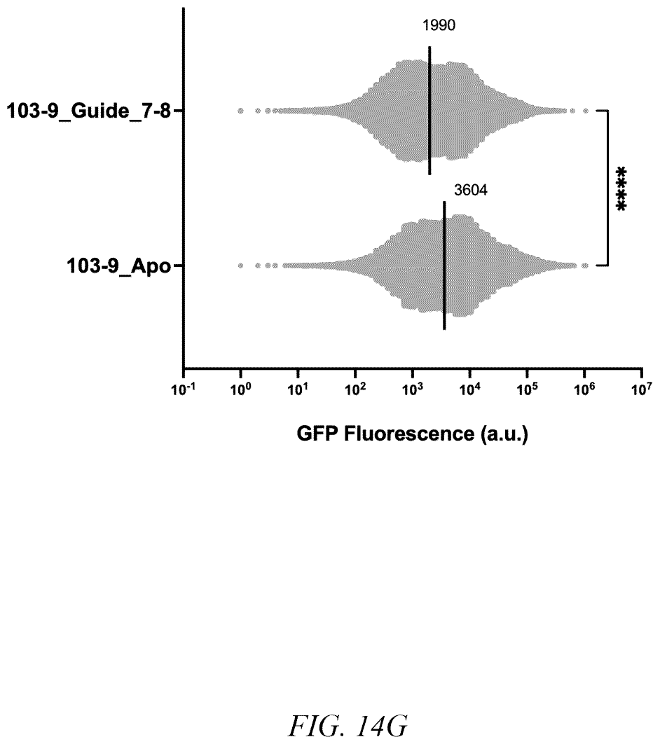

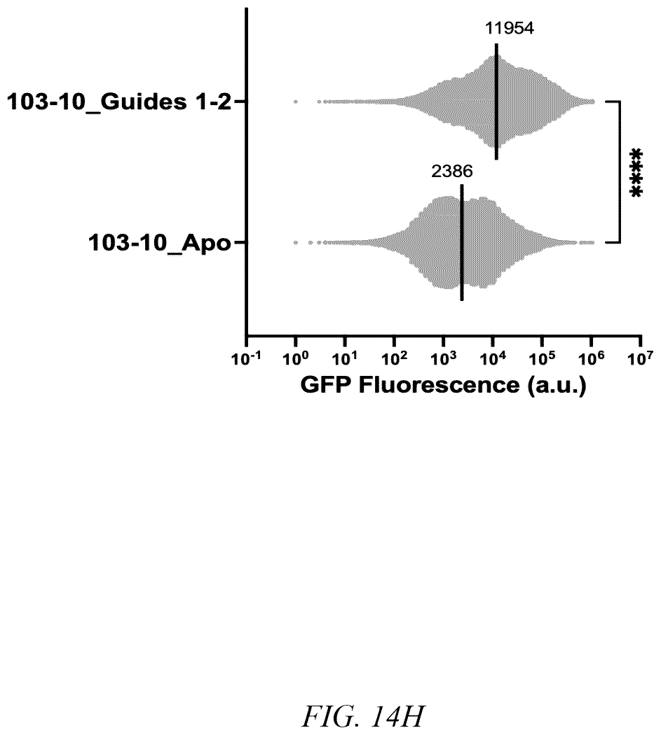

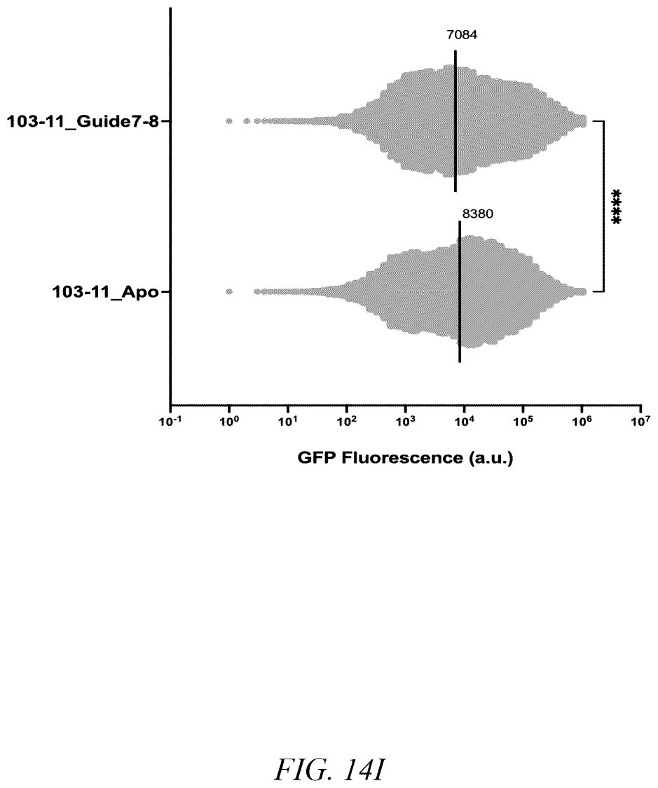

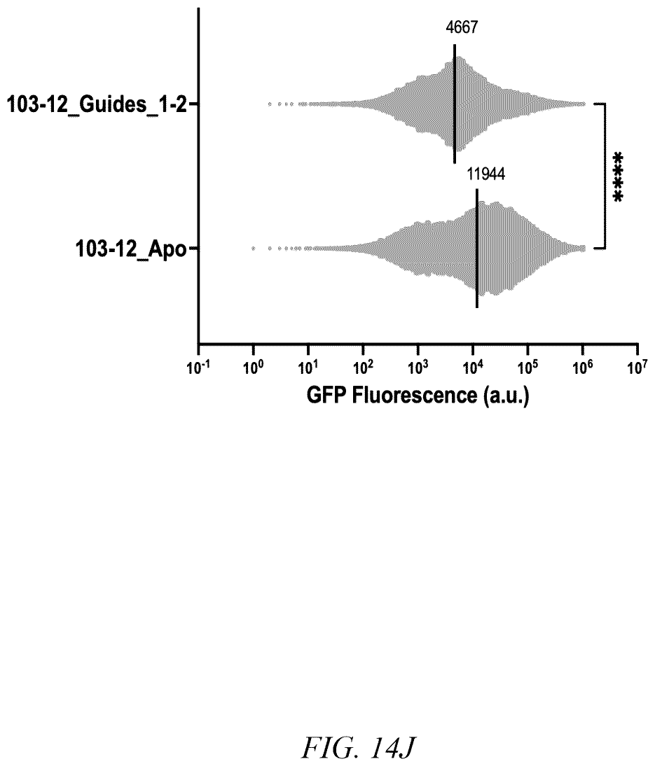

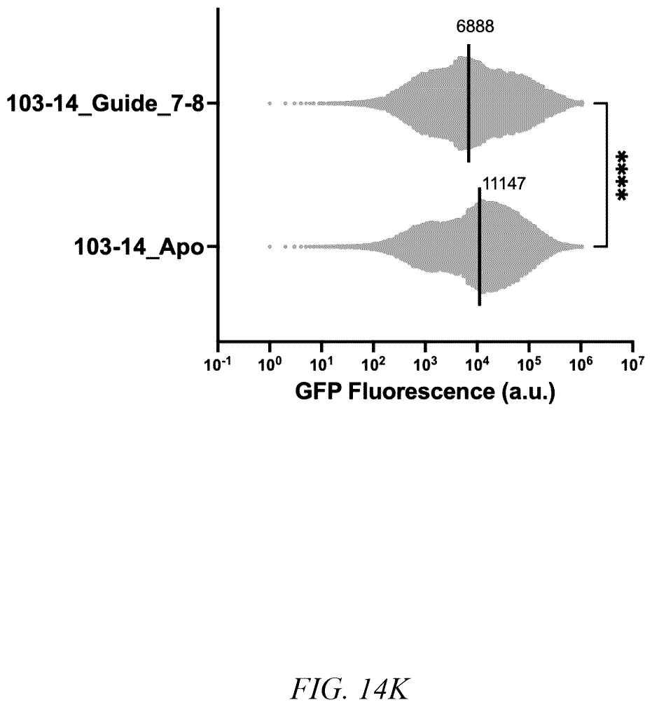

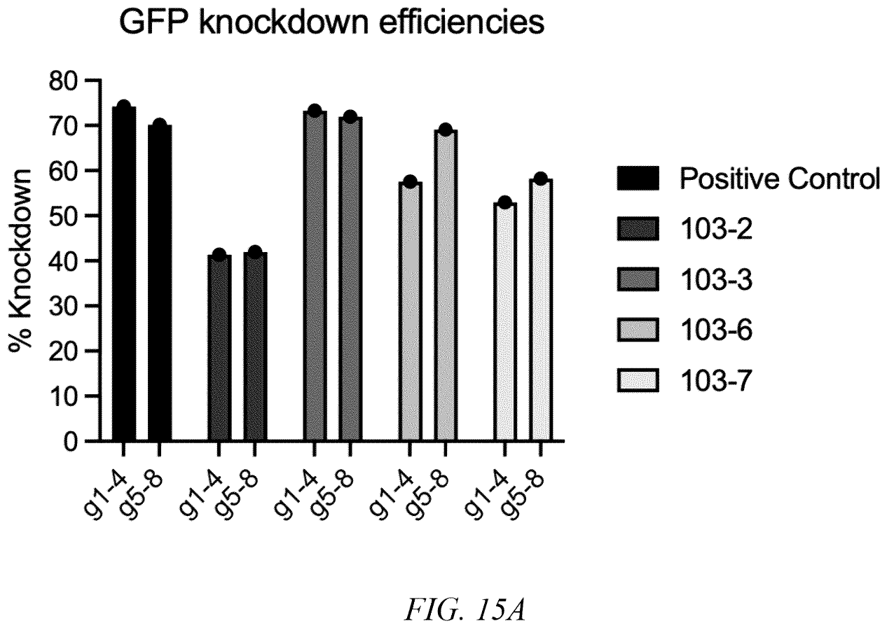

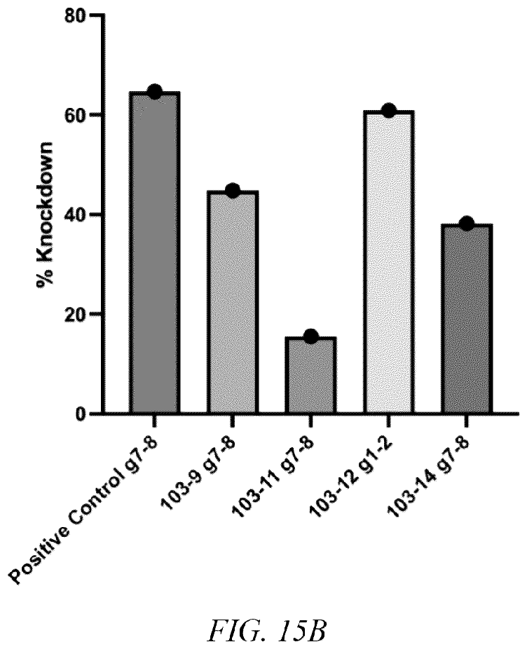

MG103-3 had the highest level of GFP knockdown (FIG. 14C and FIG. 15A), followed by MG103-6 and MG103-12. Even though the chemically synthesized guides were not tested in all conditions, it was expected to achieve similar results to plasmid-encoded guides, as validated in FIG. 12B. Altogether, it was shown that MG type VI nucleases have activity in mammalian cells and can achieve knockdown levels similar to the positive control (>70% knockdown), thus opening doors for their use in therapeutic targets of interest.

### Example 14—Nuclease Discovery and Characterization in the MG105 Family

In silico identification of novel compact type VI nucleases in the MG105 family MG105 nucleases were identified using the bioinformatics methods described in Example 13.

### In Vitro RNA Nuclease Activity Demonstrated by GFP Fluorescence Assay

Following a similar protocol as described in Example 13, in vitro cleavage activity of novel nucleases from the MG105 family (FIG. 4 and FIG. 7) were tested. MG105-targeted deGFP cis-cleavage resulted in 97.7% decrease in fluorescence compared to its apo reaction.

Trans-cleavage was quantified by taking the max fluorescence measurements of each reaction. For MG105-1, not enough data points were collected for a proper fit of the data to a plateau followed by one phase exponential decay. Instead, the Apo max fluorescence signal was subtracted from each condition then divided by the apo max fluorescence signal and multiplied by 100 (FIG. 8 and FIG. 9).

crRNA Processing Determined by RNAseq

Reads mapping to the active MG105-1 minimal array showed trimming of 10 nucleotides on the 5′ end of the spacer while leaving a 36-nucleotide repeat (FIG. 10C; SEQ ID NO: 60). This suggests that the active crRNA has a spacer on the 5′ end of the repeat.

### Mammalian Cell Activity (Prophetic)

The ueGFP cell line is used to show proof of concept of GFP knockdown using the MG105 family. Following similar protocols as above, the mammalian cellular activity of members of this family is demonstrated by analyzing GFP levels by flow cytometry. Enzymes achieving GFP repression higher than 50% are expected.

TABLE 2
Key to protein and nucleic acid sequences referred to herein

| | SEQ | | | | |
|---|---|---|---|---|---|
| Cat. | ID: | Description | Type | Organism | Other Information |
| MG105 effectors | 1 | MG105 -1 Effector | protein | unknown | uncultivated organism |
| MG105 effectors | 2 | MG105 -2 Effector | protein | unknown | uncultivated organism |
| MG103 effectors | 3 | MG103-1 Effector | protein | unknown | uncultivated organism |
| MG103 effectors | 4 | MG103-2 Effector | protein | unknown | uncultivated organism |
| MG103 effectors | 5 | MG103-3 Effector | protein | unknown | uncultivated organism |
| MG103 effectors | 6 | MG103-4 Effector | protein | unknown | uncultivated organism |
| MG103 effectors | 7 | MG103-6 Effector | protein | unknown | uncultivated organism |
| MG103 effectors | 8 | MG103-7 Effector | protein | unknown | uncultivated organism |
| MG103 effectors | 9 | MG103-8 Effector | protein | unknown | uncultivated organism |
| MG103 effectors | 10 | MG103-9 Effector | protein | unknown | uncultivated organism |
| MG103 effectors | 11 | MG103-10 Effector | protein | unknown | uncultivated organism |
| MG103 effectors | 12 | MG103-11 Effector | protein | unknown | uncultivated organism |
| MG103 effectors | 13 | MG103-12 Effector | protein | unknown | uncultivated organism |
| MG103 effectors | 14 | MG103-13 Effector | protein | unknown | uncultivated organism |
| MG103 effectors | 15 | MG103-14 Effector | protein | unknown | uncultivated organism |
| deGFP mRNA | 16 | cis-cleavage target | RNA | synthetic | |
| target | | | | | |
| Activator RNA | 17 | target for trans- | RNA | synthetic | |
| target | | cleavage assay | | | |
| MG103-2 Non- | 18 | 103- | DNA | synthetic | |
| Targeting | | 2_30_REV_NT_U40 | | | |
| Minimal Array | | | | | |
| MG103-2 deGFP | 19 | 103-2_MA_30_REV | DNA | synthetic | |
| Targeting | | | | | |
| Minimal Array | | | | | |
| MG103-2 deGFP | 20 | 103-2_MA_30_REV | RNA | synthetic | |
| Targeting | | | | | |
| Minimal Array | | | | | |
| Transcribed | | | | | |
| MG103-2 Repeat | 21 | 103-2 repeat | RNA | synthetic | |
| MG103-3 Non- | 22 | 103- | DNA | synthetic | |
| Targeting | | 3_MA_30_REV_U67 | | | |
| Minimal Array | | | | | |
| MG103-3 deGFP | 23 | 103- | DNA | synthetic | |
| Targeting | | 3_30_REV_NT_U40 | | | |
| Minimal Array | | | | | |
| MG103-3 deGFP | 24 | 103-3_MA_30_REV | RNA | synthetic | |
| Targeting | | | | | |
| Minimal Array | | | | | |
| Transcribed | | | | | |
| MG103-3 Non- | 25 | 103-3_MA_30_REV | RNA | synthetic | |
| Targeting | | | | | |
| Minimal Array | | | | | |
| MG103-4 Repeat | 26 | 103-3 repeat | RNA | synthetic | |
| MG103-4 deGFP | 27 | 103- | DNA | synthetic | |
| Targeting | | 4_30_REV_NT_U40 | | | |
| Minimal Array | | | | | |
| MG103-4 deGFP | 28 | 103-4_MA_30_REV | DNA | synthetic | |
| Targeting | | | | | |
| Minimal Array | | | | | |
| Transcribed | | | | | |
| MG103-4 Non- | 29 | 103-4_MA_30_REV | RNA | synthetic | |
| Targeting | | | | | |
| Minimal Array | | | | | |
| MG103-4 Repeat | 30 | 103-4 repeat | RNA | synthetic | |
| MG103-6 | 31 | 103- | DNA | synthetic | |
| Activator RNA | | 6_MA_30_REV_U67 | | | |
| Targeting | | | | | |
| Minimal Array | | | | | |
| MG103-6 deGFP | 32 | 103- | DNA | synthetic | |
| Targeting | | 6_30_REV_NT_U40 | | | |
| Minimal Array | | | | | |
| MG103-6 deGFP | 33 | 103-6_MA_30_REV | DNA | synthetic | |
| Targeting | | | | | |
| Minimal Array | | | | | |
| Transcribed | | | | | |
| MG103-6 Non- | 34 | 103-6_MA_30_REV | RNA | synthetic | |
| Targeting | | | | | |
| Minimal Array | | | | | |
| MG103-6 Repeat | 35 | 103-6 repeat | RNA | synthetic | |
| MG103-6 | 36 | Processed crRNA 21 nt | RNA | synthetic | |
| Processed crRNA | | spacer | | | |
| MG103-6 | 37 | Processed crRNA 26 nt | RNA | synthetic | |
| Processed crRNA | | spacer | | | |
| MG103-9 deGFP | 38 | 103- | DNA | synthetic | |
| Targeting | | 9_30_FWD_NT_U40 | | | |
| Minimal Array | | | | | |
| MG103-9 deGFP | 39 | 103-9_MA_30_FWD | DNA | synthetic | |
| Targeting | | | | | |
| Minimal Array | | | | | |
| Transcribed | | | | | |
| MG103-9 Non- | 40 | 103-9_MA_30_FWD | RNA | synthetic | |
| Targeting | | | | | |
| Minimal Array | | | | | |
| MG103-9 Repeat | 41 | 103-9 repeat | RNA | synthetic | |
| MG103-10 | 42 | 103- | DNA | synthetic | |
| Activator RNA | | 10_MA_30_FWD_U67 | | | |
| Targeting | | | | | |
| Minimal Array | | | | | |
| MG103-10 deGFP | 43 | 103- | DNA | synthetic | |
| Targeting | | 10_30_FWD_NT_U40 | | | |
| Minimal Array | | | | | |
| MG103-10 deGFP | 44 | 103-10_MA_30_FWD | DNA | synthetic | |
| Targeting | | | | | |
| Minimal Array | | | | | |
| Transcribed | | | | | |
| MG103-10 Non- | 45 | 103-10_MA_30_FWD | RNA | synthetic | |
| Targeting | | | | | |
| Minimal Array | | | | | |
| MG103-10 Repeat | 46 | 103-10 repeat | RNA | synthetic | |
| MG103-11 deGFP | 47 | 103- | DNA | synthetic | |
| Targeting | | 11_30_FWD_NT_U40 | | | |
| Minimal Array | | | | | |
| MG103-11 deGFP | 48 | 103-11_MA_30_FWD | DNA | synthetic | |
| Targeting | | | | | |
| Minimal Array | | | | | |
| Transcribed | | | | | |
| MG103-11 Non- | 49 | 103-11_MA_30_FWD | RNA | synthetic | |
| Targeting | | | | | |
| Minimal Array | | | | | |
| MG103-11 Repeat | 50 | 103-11 repeat | RNA | synthetic | |
| MG103-12 deGFP | 51 | 103- | DNA | synthetic | |
| Targeting | | 12_30_FWD_NT_U40 | | | |
| Minimal Array | | | | | |
| MG103-12 deGFP | 52 | 103-12_MA_30_FWD | DNA | synthetic | |
| Targeting | | | | | |
| Minimal Array | | | | | |
| Transcribed | | | | | |
| MG103-12 Non- | 53 | 103-12_MA_30_FWD | RNA | synthetic | |
| Targeting | | | | | |
| Minimal Array | | | | | |
| MG103-12 Repeat | 54 | 103-12 repeat | RNA | synthetic | |
| MG103-12 | 55 | Processed crRNA 21 nt | RNA | synthetic | |
| Processed crRNA | | spacer | | | |
| MG105-1 | 56 | 105- | DNA | synthetic | |
| Activator RNA | | 1_MA_30_REV_U67 | | | |
| Targeting | | | | | |
| Minimal Array | | | | | |
| MG105-1 deGFP | 57 | 105- | DNA | synthetic | |
| Targeting | | 1_MA_30_REV_NT_U40 | | | |
| Minimal Array | | | | | |
| MG105-1 deGFP | 58 | 105-1_MA_30 REV | DNA | synthetic | |
| Targeting | | | | | |
| Minimal Array | | | | | |
| Transcribed | | | | | |
| MG105-1 Non- | 59 | 105-1_MA_30_REV | RNA | synthetic | |
| Targeting | | | | | |
| Minimal Array | | | | | |
| MG105-1 Repeat | 60 | 105-1 repeat | RNA | synthetic | |
| MG105-1 | 61 | Processed crRNA 20 nt | RNA | synthetic | |
| Processed crRNA | | spacer | | | |
| MG103 effectors | 62 | MG103-15 effector | protein | unknown | uncultivated organism |
| MG103 effectors | 63 | MG103-16 effector | protein | unknown | uncultivated organism |
| MG103 effectors | 64 | MG103-17 effector | protein | unknown | uncultivated organism |
| MG103 effectors | 65 | MG103-18 effector | protein | unknown | uncultivated organism |
| MG103 effectors | 66 | MG103-19 effector | protein | unknown | uncultivated organism |
| MG103 effectors | 67 | MG103-20 effector | protein | unknown | uncultivated organism |
| MG103 effectors | 68 | MG103-21 effector | protein | unknown | uncultivated organism |
| MG103 effectors | 69 | MG103-22 effector | protein | unknown | uncultivated organism |
| MG103 effectors | 70 | MG103-23 effector | protein | unknown | uncultivated organism |
| MG103 effectors | 71 | MG103-24 effector | protein | unknown | uncultivated organism |
| MG103 effectors | 72 | MG103-25 effector | protein | unknown | uncultivated organism |
| MG103 effectors | 73 | MG103-26 effector | protein | unknown | uncultivated organism |
| MG103 effectors | 74 | MG103-27 effector | protein | unknown | uncultivated organism |
| MG103 effectors | 75 | MG103-28 effector | protein | unknown | uncultivated organism |
| MG103 effectors | 76 | MG103-29 effector | protein | unknown | uncultivated organism |
| MG103 effectors | 77 | MG103-30 effector | protein | unknown | uncultivated organism |
| MG103 effectors | 78 | MG103-31 effector | protein | unknown | uncultivated organism |
| MG103 effectors | 79 | MG103-32 effector | protein | unknown | uncultivated organism |
| MG103 effectors | 80 | MG103-33 effector | protein | unknown | uncultivated organism |
| MG103 effectors | 81 | MG103-34 effector | protein | unknown | uncultivated organism |
| MG103 effectors | 82 | MG103-35 effector | protein | unknown | uncultivated organism |
| MG103 effectors | 83 | MG103-36 effector | protein | unknown | uncultivated organism |
| MG103 effectors | 84 | MG103-37 effector | protein | unknown | uncultivated organism |
| ueGFP | 85 | GFP-PEST reporter | protein | synthetic | |
| sgRNA 103-2_non | 86 | Non-Targeting | RNA | synthetic | |
| targeting | | Chemically | | | |
| | | Synthesized RNA | | | |
| | | Guide for 103-2 | | | |
| sgRNA 103-3_non | 87 | Non-Targeting | RNA | synthetic | |
| targeting | | Chemically | | | |
| | | Synthesized RNA | | | |
| | | Guide for 103-3 | | | |
| sgRNA 103-6_non | 88 | Non-Targeting | RNA | synthetic | |
| targeting | | Chemically | | | |
| | | Synthesized RNA | | | |
| | | Guide for 103-6 | | | |
| sgRNA 103-7_non | 89 | Non-Targeting | RNA | synthetic | |
| targeting | | Chemically | | | |
| | | Synthesized RNA | | | |
| | | Guide for 103-7 | | | |
| 103-2_spacers 1_2 | 90 | Non-processed spacer | RNA | synthetic | |
| | | array with 103-2 | | | |
| | | repeats targeting eGFP | | | |
| | | at positions 1 and 2 | | | |
| 103-2_spacers 3_4 | 91 | Non-processed spacer | RNA | synthetic | |
| | | array with 103-2 | | | |
| | | repeats targeting eGFP | | | |
| | | at positions 3 and 4 | | | |
| 103-2_spacers 5_6 | 92 | Non-processed spacer | RNA | synthetic | |
| | | array with 103-2 | | | |
| | | repeats targeting eGFP | | | |
| | | at positions 5 and 6 | | | |
| 103-2_spacers 7_8 | 93 | Non-processed spacer | RNA | synthetic | |
| | | array with 103-2 | | | |
| | | repeats targeting eGFP | | | |
| | | at positions 7 and 8 | | | |
| 103-3_spacers 1_2 | 94 | Non-processed spacer | RNA | synthetic | |
| | | array with 103-3 | | | |
| | | repeats targeting eGFP | | | |
| | | at positions 1 and 2 | | | |
| 103-3_spacers 3_4 | 95 | Non-processed spacer | RNA | synthetic | |
| | | array with 103-3 | | | |
| | | repeats targeting eGFP | | | |
| | | at positions 3 and 4 | | | |
| 103-3_spacers 5_6 | 96 | Non-processed spacer | RNA | synthetic | |
| | | array with 103-3 | | | |
| | | repeats targeting eGFP | | | |
| | | at positions 5 and 6 | | | |
| 103-3_spacers 7_8 | 97 | Non-processed spacer | RNA | synthetic | |
| | | array with 103-3 | | | |
| | | repeats targeting eGFP | | | |
| | | at positions 7 and 8 | | | |
| 103-6_spacers 1_2 | 98 | Non-processed spacer | RNA | synthetic | |
| | | array with 103-6 | | | |
| | | repeats targeting eGFP | | | |
| | | at positions 1 and 2 | | | |
| 103-6_spacers 3_4 | 99 | Non-processed spacer | RNA | synthetic | |
| | | array with 103-6 | | | |
| | | repeats targeting eGFP | | | |
| | | at positions 3 and 4 | | | |
| 103-6_spacers 5_6 | 100 | Non-processed spacer | RNA | synthetic | |
| | | array with 103-6 | | | |
| | | repeats targeting eGFP | | | |
| | | at positions 5 and 6 | | | |
| 103-6_spacers 7_8 | 101 | Non-processed spacer | RNA | synthetic | |
| | | array with 103-6 | | | |
| | | repeats targeting eGFP | | | |
| | | at positions 7 and 8 | | | |
| 103-7_spacers 1_2 | 102 | Non-processed spacer | RNA | synthetic | |
| | | array with 103-7 | | | |
| | | repeats targeting eGFP | | | |
| | | at positions 1 and 2 | | | |
| 103-7_spacers 3_4 | 103 | Non-processed spacer | RNA | synthetic | |
| | | array with 103-7 | | | |
| | | repeats targeting eGFP | | | |
| | | at positions 3 and 4 | | | |
| 103-7_spacers 5_6 | 104 | Non-processed spacer | RNA | synthetic | |
| | | array with 103-7 | | | |
| | | repeats targeting eGFP | | | |
| | | at positions 5 and 6 | | | |
| 103-7_spacers 7_8 | 105 | Non-processed spacer | RNA | synthetic | |
| | | array with 103-7 | | | |
| | | repeats targeting eGFP | | | |
| | | at positions 7 and 8 | | | |
| 103- | 106 | DNA encoding for non- | DNA | synthetic | |
| 2_Plasmid_Spacer_Array_1-4 | | processed guide and | | | |
| | | repeat array for 103-2 | | | |
| | | guides 1, 2, 3, and 4 | | | |
| | | targeting eGFP cloned | | | |
| | | into and expressed by | | | |
| | | plasmid. (includes | | | |
| | | restriction digest | | | |
| | | overhangs) | | | |
| 103- | 107 | DNA encoding for non- | DNA | synthetic | |
| 2_Plasmid_Spacer_Array_5-8 | | processed guide and | | | |
| | | repeat array for 103-2 | | | |
| | | guides 5, 6, 7, and 8 | | | |
| | | targeting eGFP cloned | | | |
| | | into and expressed by | | | |
| | | plasmid. (includes | | | |
| | | restriction digest | | | |
| | | overhangs) | | | |
| 103- | 108 | DNA encoding for non- | DNA | synthetic | |
| 3_Plasmid_Spacer_Array_1-4 | | processed guide and | | | |
| | | repeat array for 103-3 | | | |
| | | guides 1, 2, 3, and 4 | | | |
| | | targeting eGFP cloned | | | |
| | | into and expressed by | | | |
| | | plasmid. (includes | | | |
| | | restriction digest | | | |
| | | overhangs) | | | |
| 103- | 109 | DNA encoding for non- | DNA | synthetic | |
| 3_Plasmid_Spacer_Array_5-8 | | processed guide and | | | |
| | | repeat array for 103-3 | | | |
| | | guides 5, 6, 7, and 8 | | | |
| | | targeting eGFP cloned | | | |
| | | into and expressed by | | | |
| | | plasmid. (includes | | | |
| | | restriction digest | | | |
| | | overhangs) | | | |
| 103- | 110 | DNA encoding for non- | DNA | synthetic | |
| 6_Plasmid_Spacer_Array_1-4 | | processed guide and | | | |
| | | repeat array for 103-6 | | | |
| | | guides 1, 2, 3, and 4 | | | |
| | | targeting eGFP cloned | | | |
| | | into and expressed by | | | |
| | | plasmid. (includes | | | |
| | | restriction digest | | | |
| | | overhangs) | | | |
| 103- | 111 | DNA encoding for non- | DNA | synthetic | |
| 6_Plasmid_Spacer_Array_5-8 | | processed guide and | | | |
| | | repeat array for 103-6 | | | |
| | | guides 5, 6, 7, and 8 | | | |
| | | targeting eGFP cloned | | | |
| | | into and expressed by | | | |
| | | plasmid. (includes | | | |
| | | restriction digest | | | |
| | | overhangs) | | | |
| 103- | 112 | DNA encoding for non- | DNA | synthetic | |
| 7_Plasmid_Spacer_Array_1-4 | | processed guide and | | | |
| | | repeat array for 103-7 | | | |
| | | guides 1, 2, 3, and 4 | | | |
| | | targeting eGFP cloned | | | |
| | | into and expressed by | | | |
| | | plasmid. (includes | | | |
| | | restriction digest | | | |
| | | overhangs) | | | |
| 103- | 113 | DNA encoding for non- | DNA | synthetic | |
| 7_Plasmid_Spacer_Array_5-8 | | processed guide and | | | |
| | | repeat array for 103-7 | | | |
| | | guides 5, 6, 7, and 8 | | | |
| | | targeting eGFP cloned | | | |
| | | into and expressed by | | | |
| | | plasmid. (includes | | | |
| | | restriction digest | | | |
| | | overhangs) | | | |
| Anti_ueGFP_Spacer 1 | 114 | Spacer sequence that | DNA | synthetic | |
| | | targets ueGFP | | | |
| Anti_ueGFP_Spacer 2 | 115 | Spacer sequence that | DNA | synthetic | |
| | | targets ueGFP | | | |
| Anti_ueGFP_Spacer 3 | 116 | Spacer sequence that | DNA | synthetic | |
| | | targets ueGFP | | | |
| Anti_ueGFP_Spacer 4 | 117 | Spacer sequence that | DNA | synthetic | |
| | | targets ueGFP | | | |
| Anti_ueGFP_Spacer 5 | 118 | Spacer sequence that | DNA | synthetic | |
| | | targets ueGFP | | | |
| Anti_ueGFP_Spacer 6 | 119 | Spacer sequence that | DNA | synthetic | |
| | | targets ueGFP | | | |
| Anti_ueGFP_Spacer 7 | 120 | Spacer sequence that | DNA | synthetic | |
| | | targets ueGFP | | | |
| Anti_ueGFP_Spacer 8 | 121 | Spacer sequence that | DNA | synthetic | |
| | | targets ueGFP | | | |
| 103-2_Repeat | 122 | Repeat sequence | DNA | synthetic | |
| | | identified by the Type | | | |
| | | VI 103-2 effector | | | |
| | | protein | | | |
| 103-3_Repeat | 123 | Repeat sequence | DNA | synthetic | |
| | | identified by the Type | | | |
| | | VI 103-3 effector | | | |
| | | protein | | | |
| 103-6_Repeat | 124 | Repeat sequence | DNA | synthetic | |
| | | identified by the Type | | | |
| | | VI 103-6 effector | | | |
| | | protein | | | |
| 103-7_Repeat | 125 | Repeat sequence | DNA | synthetic | |
| | | identified by the Type | | | |
| | | VI 103-7 effector | | | |
| | | protein | | | |
| MG103 effectors | 126 | MG103- | DNA | unknown | Mammalian codon |
| | | 2_codon_optimized | | | optimized |
| MG103 effectors | 127 | MG103- | DNA | unknown | Mammalian codon |
| | | 3_codon_optimized | | | optimized |
| MG103 effectors | 128 | MG103- | DNA | unknown | Mammalian codon |
| | | 6_codon_optimized | | | optimized |
| MG103 effectors | 129 | MG103- | DNA | unknown | Mammalian codon |
| | | 7_codon_optimized | | | optimized |
| MG103 effectors | 130 | MG103- | DNA | unknown | Mammalian codon |
| | | 9_codon_optimized | | | optimized |
| MG103 effectors | 131 | MG103- | DNA | unknown | Mammalian codon |
| | | 10_codon_optimized | | | optimized |
| MG103 effectors | 132 | MG103- | DNA | unknown | Mammalian codon |
| | | 11_codon_optimized | | | optimized |
| MG103 effectors | 133 | MG103- | DNA | unknown | Mammalian codon |
| | | 12_codon_optimized | | | optimized |
| MG103 effectors | 134 | MG103- | DNA | unknown | Mammalian codon |
| | | 14_codon_optimized | | | optimized |
| MG103- | 135 | Chemically synthesized | RNA | synthetic | |
| 9_Guide1_2_TypeVI | | guide array with | | | |
| | | guides 1 and 2 for 103- | | | |
| | | 9 | | | |
| MG103- | 136 | Chemically synthesized | RNA | synthetic | |
| 10_Guide1_2_TypeVI | | guide array with | | | |
| | | guides 1 and 2 for 103- | | | |
| | | 10 | | | |
| MG103- | 137 | Chemically synthesized | RNA | synthetic | |
| 11_Guide1_2_TypeVI | | guide array with | | | |
| | | guides 1 and 2 for 103- | | | |
| | | 11 | | | |
| MG103- | 138 | Chemically synthesized | RNA | synthetic | |
| 12_Guide1_2_TypeVI | | guide array with | | | |
| | | guides 1 and 2 for 103- | | | |
| | | 12 | | | |
| MG103- | 139 | Chemically synthesized | RNA | synthetic | |
| 14_Guide1_2_TypeVI | | guide array with | | | |
| | | guides 1 and 2 for 103- | | | |
| | | 14 | | | |
| MG103- | 140 | Chemically synthesized | RNA | synthetic | |
| 9_Guide3_4_TypeVI | | guide array with | | | |
| | | guides 3 and 4 for 103- | | | |
| | | 9 | | | |
| MG103- | 141 | Chemically synthesized | RNA | synthetic | |
| 10_Guide3_4_TypeVI | | guide array with | | | |
| | | guides 3 and 4 for 103- | | | |
| | | 10 | | | |
| MG103- | 142 | Chemically synthesized | RNA | synthetic | |
| 11_Guide3_4_TypeVI | | guide array with | | | |
| | | guides 3 and 4 for 103- | | | |
| | | 11 | | | |
| MG103- | 143 | Chemically synthesized | RNA | synthetic | |
| 12_Guide3_4_TypeVI | | guide array with | | | |
| | | guides 3 and 4 for 103- | | | |
| | | 12 | | | |
| MG103- | 144 | Chemically synthesized | RNA | synthetic | |
| 14_Guide3_4_TypeVI | | guide array with | | | |
| | | guides 3 and 4 for 103- | | | |
| | | 14 | | | |
| MG103- | 145 | Chemically synthesized | RNA | synthetic | |
| 9_Guide5_6_TypeVI | | guide array with | | | |
| | | guides 5 and 6 for 103- | | | |
| | | 9 | | | |
| MG103- | 146 | Chemically synthesized | RNA | synthetic | |
| 10_Guide5_6_TypeVI | | guide array with | | | |
| | | guides 5 and 6 for 103- | | | |
| | | 10 | | | |
| MG103- | 147 | Chemically synthesized | RNA | synthetic | |
| 11_Guide5_6_TypeVI | | guide array with | | | |
| | | guides 5 and 6 for 103- | | | |
| | | 11 | | | |
| MG103- | 148 | Chemically synthesized | RNA | synthetic | |
| 12_Guide5_6_TypeVI | | guide array with | | | |
| | | guides 5 and 6 for 103- | | | |
| | | 12 | | | |
| MG103- | 149 | Chemically synthesized | RNA | synthetic | |
| 14_Guide5_6_TypeVI | | guide array with | | | |
| | | guides 5 and 6 for 103- | | | |
| | | 14 | | | |
| MG103- | 150 | Chemically synthesized | RNA | synthetic | |
| 9_Guide_7_8_TypeVI | | guide array with | | | |
| | | guides 7 and 8 for 103- | | | |
| | | 9 | | | |
| MG103- | 151 | Chemically synthesized | RNA | synthetic | |
| 10_Guide_7_8_TypeVI | | guide array with | | | |
| | | guides 7 and 8 for 103- | | | |
| | | 10 | | | |
| MG103- | 152 | Chemically synthesized | RNA | synthetic | |
| 11_Guide_7_8_TypeVI | | guide array with | | | |
| | | guides 7 and 8 for 103- | | | |
| | | 11 | | | |
| MG103- | 153 | Chemically synthesized | RNA | synthetic | |
| 12_Guide_7_8_Type VI | | guide array with | | | |
| | | guides 7 and 8 for 103- | | | |
| | | 12 | | | |
| MG103- | 154 | Chemically synthesized | RNA | synthetic | |
| 14_Guide_7_8_TypeVI | | guide array with | | | |
| | | guides 7 and 8 for 103- | | | |
| | | 14 | | | |

TABLE 3
Additional Protein and nucleic acid sequences referred to herein

| | SEQ | | | | Other | |
|---|---|---|---|---|---|---|
| Cat. | ID: | Description | Type | Organism | Information | Sequence |
| MG106 | 171 | MG106-1 | protein | unknown | uncultivated | MGAIENKHIFAAYANL |
| effectors | | Effector | | | organism | AIDGLIKTLNFIAKKL |
| | | | | | | DTQKQLSSWDIKHVIT |
| | | | | | | LIDSIFDQNPQNNLEQ |
| | | | | | | VVEGYLPWIKPIIEMK |
| | | | | | | TPKKGERQSDKLCIEY |
| | | | | | | KTIITAFASLLNDVRN |
| | | | | | | YYTHYYHDPICIYPGG |
| | | | | | | YDIPSSLNCIYDSAIN |
| | | | | | | IIKERFQAEEKEMEHL |
| | | | | | | RRYTRKKGRVVLKTED |
| | | | | | | DHFYYTLANNNDLSEK |
| | | | | | | GYAFFISMFLERKYSY |
| | | | | | | LFLKKLSGFKRGDSLQ |
| | | | | | | YRLTLEVFTALSTKPP |
| | | | | | | VERLRTTKDTKQDRAL |
| | | | | | | DILNELSRIPIELYQT |
| | | | | | | LEPKYREMYNETLQPT |
| | | | | | | DAEDPYGLPDRSRIRF |
| | | | | | | RSRFEAFALHFLDKQA |
| | | | | | | DFKEIGFYTYLGNYFH |
| | | | | | | NGYQKTRVDRETKDRY |
| | | | | | | INFQLAGFCKNIQDIS |
| | | | | | | AKKLSEALNVKSIDIS |
| | | | | | | TDSIPDINSFEPYLVQ |
| | | | | | | STPHYIVNGNNIGIKV |
| | | | | | | LPEGKDTYPTIDEKGA |
| | | | | | | KMPIADFWLSKYELPA |
| | | | | | | MLFYTYLRNNNIHKSH |
| | | | | | | CPLSVKDIIERSIHKS |
| | | | | | | TKQKHPEERSELMLRR |
| | | | | | | VMKAIFWTDSKLNEVE |
| | | | | | | RIKSQKSAFGKRQHEI |
| | | | | | | LKAGRIAETLVRDMLW |
| | | | | | | LQPSKNNGRDKVTEPN |
| | | | | | | FQAIQVSLAYFGIRRN |
| | | | | | | DLTEIFTRAGLINSSN |
| | | | | | | PHPFLAQIGTNYTSLI |
| | | | | | | EFYIAYLKERKVYFSR |
| | | | | | | IQKKILQGKLNIQCHP |
| | | | | | | LRDLQREPNKPQDKEE |
| | | | | | | AIFLPRGLFNEAIINC |
| | | | | | | LKKSKLKQLIESPTRE |
| | | | | | | KSPALNVSYLIQNYFR |
| | | | | | | TYFEDQSQEFYAQPRN |
| | | | | | | YRLFDKLSPNKGKSKS |
| | | | | | | YLSLEQRIKKMEELRP |
| | | | | | | SKIPVAEANKLLEKED |
| | | | | | | RLYRKNYNEICDNESI |
| | | | | | | IRLYQIQDILLFMMTK |
| | | | | | | EYLPSDLYNRINKYKL |
| | | | | | | ENVKGILNERVSYLID |
| | | | | | | LNPLKIQGEDIKIKDY |
| | | | | | | GKLFYIHHDTRINSLN |
| | | | | | | KVLSKVKRNNSISSSV |
| | | | | | | KIQPYENYKRECLDFE |
| | | | | | | EAQIQUIPIIHSFEIA |
| | | | | | | MVSMFPDLKKATPGNY |
| | | | | | | YDFNELITEYEKRTKQ |
| | | | | | | KIDSSFLIKTRNMFLH |
| | | | | | | DKYEAECIKEISDDFV |
| | | | | | | YAKKIIAEFKMKIENI |
| | | | | | | KLEDLSNDSSA |
| MG106 | 172 | MG106-2 | protein | unknown | uncultivated | MGAIENKHIFAAYANL |
| effectors | | Effector | | | organism | AIDGLIKTLNFIAKKL |
| | | | | | | DTQKQLSSWDIKHVIT |
| | | | | | | LIDSIFDQNPQNNLEQ |
| | | | | | | VVEGYLPWIKPIIEMK |
| | | | | | | TPKKGERQSDKLCIEY |
| | | | | | | KTIITAFASLLNDVRN |
| | | | | | | YYTHYYHDPICIYPRG |
| | | | | | | YDIPSSLNCIYDSAIN |
| | | | | | | IIKERFQAEEKEMEHL |
| | | | | | | RNYTLVNNNGLSEKGY |
| | | | | | | AFFISKFLERKYSYLF |
| | | | | | | LKKLSGFKRGDSLQYR |
| | | | | | | LTLEVFTALSTKPPVE |
| | | | | | | RLRTTKDTKQDRALDI |
| | | | | | | LNELSRIPIELYQTLE |
| | | | | | | PKYREMYNETLQPTDA |
| | | | | | | EDPYGLPDRSRIRFRS |
| | | | | | | RFEAFALHFLDKQADF |
| | | | | | | KEIGFYTYLGNYFHNG |
| | | | | | | YQKTRVDRETKDRYIN |
| | | | | | | FQLAGFCKNIQDISAK |
| | | | | | | KLSEALNVKSIDISTD |
| | | | | | | SIPDINSFEPYLVQST |
| | | | | | | PHYIVNGNNIGIKVLP |
| | | | | | | EGKDTYPTIDEKGAKM |
| | | | | | | PIADFWLSKYELPAML |
| | | | | | | FYTYLRNNNIHKSHCP |
| | | | | | | LSVKDIIERSIHKSTK |
| | | | | | | QKHPEERSELMLRRVM |
| | | | | | | KAIFWTDSKLNEVERI |
| | | | | | | KSQKSAFGKRQHEILK |
| | | | | | | AGRIAETLVRDMLWLQ |
| | | | | | | PSKNNGRDKVTEPNFQ |
| | | | | | | AIQVSLAYFGIRRNDL |
| | | | | | | TEIFTRAGLINSSNPH |
| | | | | | | PFLAQIGTNYTSLIEF |
| | | | | | | YIAYLKERKVYFSRIQ |
| | | | | | | KKILQGKLNIQCHPLR |
| | | | | | | DLQREPNKPQEKEEAI |
| | | | | | | FLPRGLFNEAIINCLK |
| | | | | | | KSKLKHLIESPTREKS |
| | | | | | | PALNVSYLIHNYFRAY |
| | | | | | | FEDQSQEFYAQPRNYR |
| | | | | | | LFDKLSPNKGKSKSYL |
| | | | | | | SLEQRIKKMEELRPSK |
| | | | | | | IPVAEANKLLEKEDRL |
| | | | | | | YRKNYNEICDNESIIR |
| | | | | | | LYQIQDILLFMMTKEY |
| | | | | | | LPSDLYNRINKYKLEN |
| | | | | | | VKGILNERVSYLIDLN |
| | | | | | | PLKIQGEDIKIKDYGK |
| | | | | | | LFYIHHDTRISSLNKV |
| | | | | | | LSKVKRNNSISSSVKI |
| | | | | | | QPYENYKRECLDFEEA |
| | | | | | | QIQIIPIIHSFEIAMV |
| | | | | | | SMFPDLKKATPGNYYD |
| | | | | | | FNELITEYEKRTKQKI |
| | | | | | | DSSFLIKTRNMFLHDK |
| | | | | | | YEAECIKEISDDFVYA |
| | | | | | | KKIIAEFKMKIENIKL |
| | | | | | | EDLSNDSSA |
| MG106-1 | 173 | 106- | DNA | synthetic | | ATACCTGAAACAAAAC |
| deGFP | | 1_MA_30_REV | | | | CCATCGTACGGCCAAG |
| Targeting | | | | | | GAAGTCTCCAATAACT |
| Minimal | | | | | | GTGATCCACCACAAGC |
| Array | | | | | | GCCAGGGTTTTCCCAG |
| | | | | | | TCACGACGTTGTAAAA |
| | | | | | | CGACGGCCAGTCATGC |
| | | | | | | ATAATCCGCACGCATC |
| | | | | | | TGGAATAAGGAAGTGC |
| | | | | | | CATTCCGCCTGACCTT |
| | | | | | | AATACGACTCACTATA |
| | | | | | | GGTTGTATTAGCCTTT |
| | | | | | | AGTTTGAAAGGTAAAA |
| | | | | | | ACAACCCGTCCAGCTC |
| | | | | | | GACCAGGATGGGAACA |
| | | | | | | ACGGTTGTATTAGCCT |
| | | | | | | TTAGTTTGAAAGGTAA |
| | | | | | | AAACAACAGGCTAGGT |
| | | | | | | GGAGGCTCAGTG |
| MG106-2 | 174 | 106- | DNA | synthetic | | ATACCTGAAACAAAAC |
| deGFP | | 2_MA_30_REV | | | | CCATCGTACGGCCAAG |
| Targeting | | | | | | GAAGTCTCCAATAACT |
| Minimal | | | | | | GTGATCCACCACAAGC |
| Array | | | | | | GCCAGGGTTTTCCCAG |
| | | | | | | TCACGACGTTGTAAAA |
| | | | | | | CGACGGCCAGTCATGC |
| | | | | | | ATAATCCGCACGCATC |
| | | | | | | TGGAATAAGGAAGTGC |
| | | | | | | CATTCCGCCTGACCTT |
| | | | | | | AATACGACTCACTATA |
| | | | | | | GGTTGTATTAGCCTTT |
| | | | | | | AGTTTGAAAGGTAGAA |
| | | | | | | ACAACCCGTCCAGCTC |
| | | | | | | GACCAGGATGGGAACA |
| | | | | | | ACGGTTGTATTAGCCT |
| | | | | | | TTAGTTTGAAAGGTAG |
| | | | | | | AAACAACAGGCTAGGT |
| | | | | | | GGAGGCTCAGTG |
| MG106-1 | 175 | 106- | DNA | synthetic | | ATACCTGAAACAAAAC |
| Non- | | 1_MA_30_ | | | | CCATCGTACGGCCAAG |
| Targeting | | REV_NT_U40 | | | | GAAGTCTCCAATAACT |
| Minimal | | | | | | GTGATCCACCACAAGC |
| Array | | | | | | GCCAGGGTTTTCCCAG |
| | | | | | | TCACGACGTTGTAAAA |
| | | | | | | CGACGGCCAGTCATGC |
| | | | | | | ATAATCCGCACGCATC |
| | | | | | | TGGAATAAGGAAGTGC |
| | | | | | | CATTCCGCCTGACCTT |
| | | | | | | AATACGACTCACTATA |
| | | | | | | GGTTGTATTAGCCTTT |
| | | | | | | AGTTTGAAAGGTAAAA |
| | | | | | | ACAACTGGAGATATCT |
| | | | | | | TGAACCTTGCATCCCC |
| | | | | | | GGAGTTGTATTAGCCT |
| | | | | | | TTAGTTTGAAAGGTAA |
| | | | | | | AAACAACAGGCTAGGT |
| | | | | | | GGAGGCTCAGTG |
| MG106-2 | 176 | 106- | DNA | synthetic | | ATACCTGAAACAAAAC |
| Non- | | 2_MA_30_ | | | | CCATCGTACGGCCAAG |
| Targeting | | REV_NT_U40 | | | | GAAGTCTCCAATAACT |
| Minimal | | | | | | GTGATCCACCACAAGC |
| Array | | | | | | GCCAGGGTTTTCCCAG |
| | | | | | | TCACGACGTTGTAAAA |
| | | | | | | CGACGGCCAGTCATGC |
| | | | | | | ATAATCCGCACGCATC |
| | | | | | | TGGAATAAGGAAGTGC |
| | | | | | | CATTCCGCCTGACCTT |
| | | | | | | AATACGACTCACTATA |
| | | | | | | GGTTGTATTAGCCTTT |
| | | | | | | AGTTTGAAAGGTAGAA |
| | | | | | | ACAACTGGAGATATCT |
| | | | | | | TGAACCTTGCATCCCC |
| | | | | | | GGAGTTGTATTAGCCT |
| | | | | | | TTAGTTTGAAAGGTAG |
| | | | | | | AAACAACAGGCTAGGT |
| | | | | | | GGAGGCTCAGTG |
| MG106-1 | 177 | 106- | RNA | synthetic | | GGUUGUAUUAGCCUUU |
| deGFP | | 1_MA 30_REV | | | | AGUUUGAAAGGUAAAA |
| Targeting | | | | | | ACAACCCGUCCAGCUC |
| Minimal | | | | | | GACCAGGAUGGGAACA |
| Array | | | | | | ACGGUUGUAUUAGCCU |
| Tran- | | | | | | UUAGUUUGAAAGGUAA |
| scribed | | | | | | AAACAACAGGCUAGGU |
| | | | | | | GGAGGCUCAGUG |
| MG106-2 | 178 | 106- | DNA | synthetic | | GGTTGTATTAGCCTTT |
| deGFP | | 2_MA_30_REV | | | | AGTTTGAAAGGTAGAA |
| Targeting | | | | | | ACAACCCGTCCAGCTC |
| Minimal | | | | | | GACCAGGATGGGAACA |
| Array | | | | | | ACGGTTGTATTAGCCT |
| Tran- | | | | | | TTAGTTTGAAAGGTAG |
| scribed | | | | | | AAACAACAGGCTAGGT |
| | | | | | | GGAGGCTCAGTG |
| MG106-1 | 179 | 106-1_repeat | RNA | synthetic | | GUUGUAUUAGCCUUUA |
| Repeat | | | | | | GUUUGAAAGGUAAAAA |
| | | | | | | CAAC |
| MG106-2 | 180 | 106-2_repeat | DNA | synthetic | | GTTGTATTAGCCTTTA |
| Repeat | | | | | | GTTTGAAAGGTAGAAA |
| | | | | | | CAAC |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An engineered nuclease system comprising:
(a) an endonuclease comprising an HEPN domain, wherein said endonuclease is derived from an uncultivated microorganism; and
(b) an engineered guide ribonucleic acid structure configured to form a complex with said endonuclease comprising:
(i) a ribonucleic acid sequence configured to hybridize to a target ribonucleic acid sequence; and
(ii) a ribonucleic acid sequence configured to bind to said endonuclease.

2. The engineered nuclease system of claim 1, wherein said endonuclease comprises a sequence having at least 75% sequence identity to any one of SEQ ID NOs: 1-15 and 62-84, or a variant thereof.

3. The engineered nuclease system of claim 1, wherein said endonuclease is not a Cas9 endonuclease, a Cas14 endonuclease, a Cas12a endonuclease, a Cas12b endonuclease, a Cas12c endonuclease, a Cas12d endonuclease, a Cas12e endonuclease, a Cas13a endonuclease, a Cas13b endonuclease, a Cas13c endonuclease, or a Cas13d endonuclease.

4. The engineered nuclease system of claim 1, wherein said endonuclease has less than 80% sequence identity to a Cas13b endonuclease.

5. The engineered nuclease system of claim 1, wherein said endonuclease comprises a sequence having at least about 75% sequence identity to any one of SEQ ID NOs: 1, 4, 5, 6, 7, 8, 10, 11, 12, 13, or 15, or a variant thereof.

6. The engineered nuclease system of claim 1, wherein said engineered guide ribonucleic acid structure comprises a repeat having a least 30 continuous nucleotides having at least about 80% sequence identity to any one of SEQ ID NOs: 21, 26, 30, 35, 41, 46, 50, 54, 60, 122, 123, 124, or 125.

7. The engineered nuclease system of claim 1, wherein said ribonucleic acid sequence configured to hybridize to said target ribonucleic acid sequence comprises at least about 18 to about 26 nucleotides.

8. The engineered nuclease system of claim 6, wherein said engineered guide ribonucleic acid structure is provided as a sequence comprising:
(i) a first copy of said repeat;
(ii) said ribonucleic acid sequence configured to hybridize to said target ribonucleic acid sequence; and
(iii) a second copy of said repeat.

9. The engineered nuclease system of claim 1, wherein said engineered guide ribonucleic acid structure comprises a sequence having at least about 80% sequence identity to non-degenerate nucleotides of any one of SEQ ID NOs: 36, 37, 55, or 61.

10.-24. (canceled)

25. An engineered guide ribonucleic acid polynucleotide comprising:
(a) a ribonucleic acid (RNA)-targeting segment comprising a nucleotide sequence that is complementary to a target sequence in a target RNA molecule; and
(b) a protein-binding segment comprising two complementary stretches of nucleotides that hybridize to form a double-stranded RNA (dsRNA) duplex;
wherein said two complementary stretches of nucleotides are covalently linked to one another with intervening nucleotides, and
wherein said engineered guide ribonucleic acid polynucleotide is configured to form a complex with an endonuclease comprising a sequence having at least 75% sequence identity to any one of SEQ ID NOs: 1-15 and 62-84, or a variant thereof and target said complex to said target sequence of said target RNA molecule.

26. The engineered guide ribonucleic acid polynucleotide of claim 25, wherein said RNA-targeting segment is positioned 5′ of both of said two complementary stretches of nucleotides.

27. A deoxyribonucleic acid polynucleotide encoding the engineered guide ribonucleic acid polynucleotide of claim 25.

28.-36. (canceled)

37. A method for binding, cleaving, marking, or modifying a single-stranded ribonucleic acid polynucleotide, comprising:
contacting said single-stranded ribonucleic acid polynucleotide with a class 2, type VI endonuclease in complex with an engineered guide ribonucleic acid structure configured to bind to said endonuclease and target said class 2, type VI endonuclease to a target ribonucleic acid sequence and said single-stranded ribonucleic acid polynucleotide.

38. The method of claim 37, wherein said single-stranded ribonucleic acid polynucleotide comprises a protospacer flanking site (PFS).

39. The method of claim 38, wherein said PFS comprises GTT.

40. The method of claim 37, wherein said single-stranded ribonucleic acid polynucleotide comprises a sequence complementary to a sequence of said engineered guide ribonucleic acid structure and a PFS.

41. The method of claim 38, wherein said PFS is adjacent to said sequence complementary to said sequence of said engineered guide ribonucleic acid structure.

42. The method of claim 37, wherein said single single-stranded ribonucleic acid polynucleotide does not comprise a protospacer flanking site (PFS).

43. The method of claim 37, wherein said class 2, type VI endonuclease is not a Cas9 endonuclease, a Cas14 endonuclease, a Cas12a endonuclease, a Cas12b endonuclease, a Cas12c endonuclease, a Cas12d endonuclease, a Cas12e endonuclease, a Cas13a endonuclease, a Cas13b endonuclease, a Cas13c endonuclease, or a Cas13d endonuclease.

44. The method of claim 37, wherein said single-stranded ribonucleic acid polynucleotide is a eukaryotic, plant, fungal, mammalian, rodent, or human single-stranded ribonucleic acid polynucleotide.

45.-63. (canceled)
